(19) United States
(12) Patent Application Publication
(10) Pub. No.: US 2024/0010696 A1
(43) Pub. Date: Jan. 11, 2024

(54) IL-12 Variants and Uses Thereof

(71) Applicant: Pfizer Inc., New York, NY (US)

(72) Inventors: James Reasoner APGAR, Newton, MA (US); Javier Fernando CHAPARRO RIGGERS, San Diego, CA (US); Ling Hon Matthew CHU, San Mateo, CA (US); Tzu-Hsuan HUANG, San Diego, CA (US); Kritika MOHAN, San Diego, CA (US); Lidia MOSYAK, Newton, MA (US); Edward Derrick PASCUA, San Diego, CA (US); James Travis PATTERSON, San Diego, CA (US); Gabriel Roy STARBECK-MILLER, San Diego, CA (US); Dirk Michael ZAJONC, Poway, CA (US)

(73) Assignee: Pfizer Inc., New York, NY (US)

(21) Appl. No.: 18/335,259

(22) Filed: Jun. 15, 2023

Related U.S. Application Data
(60) Provisional application No. 63/496,545, filed on Apr. 17, 2023.
(60) Provisional application No. 63/353,241, filed on Jun. 17, 2022.

(51) Int. Cl. C07K 14/54; C07K 16/28; A61P 35/00; C12N 15/63

(52) U.S. Cl. CPC C07K 14/5434; C07K 16/2818; A61P 35/00; C12N 15/63; C07K 2317/565

(57) ABSTRACT

IL-12 variants are provided. Also provided are antibodies that specifically bind to PD1. Also provided are fusion proteins of IL-12 variants and anti-PD1 antibodies. Also provided are uses of these IL-12 variants, anti-PD1 antibodies, fusion proteins, and related compositions and methods.

### REFERENCE TO SEQUENCE LISTING

The instant application contains a Sequence Listing which has been submitted electronically in .xml format and is hereby incorporated by reference in its entirety. Said Sequence Listing, created on May 2, 2023, is named “PC072389A SEQ Listing ST.26.xml” and is 72 kilobytes in size.

### BACKGROUND

The present invention relates to interleukin 12 (IL-12) variants, and methods of making and using the variants. The present invention also provides anti-PD1 antibodies, and fusion proteins comprising such IL-12 variants and anti-PD1 antibodies. The present invention also pertains to related molecules, e.g. nucleic acids which encode such IL-12 variants, fusion proteins and antibodies, and related compositions and methods.

Interleukin 12 (IL-12) is a cytokine with multiple functions in the immune system. IL-12 is a heterodimer containing two subunits, p35 (encoded by the IL-12A gene) and p40 (encoded by the IL-12B gene). IL-12 binds and crosslinks the heterodimeric IL-12 receptor (IL-12R) chains IL-12Rβ1 and IL-12Rβ2. IL-12R is upregulated by T cell receptor (TCR) activation, thereby boosting T cell sensitivity to IL-12 stimulation. After IL-12 binds to the IL-12R STAT4 is phosphorylated (pSTAT4) and pSTAT4 promotes IL-12-dependent effects including interferon gamma (IFNg) production and cytolytic capacity of CD8 T cells, CD4 T cells, T regulatory cells, and NK cells.

Preclinical models have shown that IL-12 promotes anti-tumor immunity through direct action on T cells and NK cells, and indirect action on antigen-presenting cells in the tumor microenvironment (TME). However, preclinical and clinical studies also have shown that IL-12R agonists can have dose limiting toxicities. These studies speculated that significant toxicities were likely arising from systemic activity.

Programmed cell death protein 1 (PD1) is an important cell surface receptor that serves to dampen T cell activation signals and functions as a checkpoint molecule that limits anti-tumor immunity. Although some PD1 expression has been observed on a variety of immune cell subsets including B cells and innate immune cells, high expression of PD1 is predominantly seen on CD8 and CD4 tumor-infiltrating lymphocytes (TIL) and enriched in the TME compared to circulating T cell subsets.

Previous studies have targeted cytokines including IL-15, IL-12, and IFN toward PD1-positive cells by fusing anti-PD1 antibodies to cytokine mutein variants (partial agonists). (Xu, Y., et al., Cancer Immunol Res, 2021. 9(10): p. 1141-1157; Codarri Deak, L., et al., Nature, 2022. 610(7930): p. 161-172; Hashimoto, M., et al., Nature, 2022. 610(7930): p. 173-181. Garcin, G., et al., Nat Commun, 2014. 5: p. 3016.).

However, there is a need for improved IL-12 variants and related fusion proteins.

### SUMMARY

The present disclosure provides interleukin 12 (IL-12) variants, and methods of making and using the variants. The present invention also provides anti-PD1 antibodies, and fusion proteins comprising the IL-12 variants and anti-PD1 antibodies. In some embodiments, the IL-12 variants provided herein have reduced activity as compared to wild-type IL-12. In some embodiments, the anti-PD1 antibodies provided herein can bind to PD1 at the same time that PD1 is bound to PDL1 (e.g. the anti-PD1 antibody binds to a different location on PD1 than is bound by PDL1). In some embodiments, IL-12 variant/anti-PD1 fusion proteins provided herein have activity biased towards the tumor microenvironment (TME), instead of systemic activity. In some embodiments, IL-12 variant/anti-PD1 fusion proteins provided herein have improved an improved therapeutic index as compared to wild-type IL-12 and fusions thereof.

The present disclosure further encompasses expression of the IL-12 variants, anti-PD1 antibodies, and fusion proteins, and preparation and manufacture of compositions comprising IL-12 variants, antibodies, and fusion proteins of the disclosure, such as medicaments for the use of the IL-12 variants, antibodies, and fusion proteins.

In some embodiments, provided herein is an isolated human interleukin 12 (IL-12) variant comprising an amino acid substitution at position Y167 of SEQ ID NO: 1 (IL-12 p35 subunit) and position D93 of SEQ ID NO: 2 (IL-12 p40 subunit).

In some embodiments, provided herein is an isolated human interleukin 12 (IL-12) variant comprising the amino acid substitutions Y167A of SEQ ID NO: 1 (IL-12 p35 subunit) and D93L of SEQ ID NO: 2 (IL-12 p40 subunit).

In some embodiments, provided herein is an isolated human interleukin 12 (IL-12) variant comprising one or both of i) a polypeptide comprising the amino acid sequence of SEQ ID NO: 3 (variant IL-12 p35 subunit) and ii) a polypeptide comprising the amino acid sequence of SEQ ID NO: 4 (variant IL-12 p40 subunit).

In some embodiments, provided herein is an isolated human interleukin 12 (IL-12) variant comprising an amino acid substitution at one or more of positions: F39 of SEQ ID NO: 1 (IL-12 p35 subunit), 152 of SEQ ID NO: 1, Y167 of SEQ ID NO: 1, K85 of SEQ ID NO: 2 (IL-12 p40 subunit) and D93 of SEQ ID NO: 2.

In some embodiments, provided herein is an isolated antibody that binds to PD1 and comprises a heavy chain variable region (VH) and a light chain variable region (VL), wherein the VH CDR1 comprises the amino acid sequence of SEQ ID NOs: 9, 35, or 36, the VH CDR2 comprises the amino acid sequence of SEQ ID NO: 10 or 37, the VH CDR3 comprises the amino acid sequence of SEQ ID NO: 11, the VL CDR1 comprises the amino acid sequence of SEQ ID NO: 12, the VL CDR2 comprises the amino acid sequence of SEQ ID NO: 13, and the VL CDR3 comprises the amino acid sequence of SEQ ID NO: 14.

In some embodiments, provided herein is an isolated antibody that binds to PD1 comprising: a VH amino acid sequence comprising a VH CDR1, VH CDR2, and VH CDR3 of the amino acid sequence of SEQ ID NO. 7 and a VL CDR1, VL CDR2, and VL CDR3 of the amino acid sequence of SEQ ID NO: 8.

In some embodiments, provided herein is an isolated antibody comprising a heavy chain comprising the amino acid sequence of SEQ ID NO: 5, 51 or 52 and a light chain comprising the amino acid sequence of SEQ ID NO: 6, wherein the C-terminal lysine of SEQ ID NO: 5, 51, or 52 is optional.

In some embodiments, provided herein is an isolated antibody that binds to PD1 and comprises a heavy chain variable region (VH) and a light chain variable region (VL), wherein the VH CDR1 comprises the amino acid sequence of SEQ ID NO: 19, 35, or 36, the VH CDR2 comprises the amino acid sequence of SEQ ID NO: 20 or 37, the VH CDR3 comprises the amino acid sequence of SEQ ID NO: 21, the VL CDR1 comprises the amino acid sequence of SEQ ID NO: 22, the VL CDR2 comprises the amino acid sequence of SEQ ID NO: 23, and the VL CDR3 comprises the amino acid sequence of SEQ ID NO: 24.

In some embodiments, provided herein is an isolated antibody that binds to PD1 comprising: a VH amino acid sequence comprising a VH CDR1, VH CDR2, and VH CDR3 of the amino acid sequence of SEQ ID NO: 17 and a VL CDR1, VL CDR2, and VL CDR3 of the amino acid sequence of SEQ ID NO: 18.

In some embodiments, provided herein is an isolated antibody comprising a heavy chain comprising the amino acid sequence of SEQ ID NO: 15, 53, or 54 and a light chain comprising the amino acid sequence of SEQ ID NO: 16, wherein the C-terminal lysine of SEQ ID NO: 15, 53, or 54 is optional.

In some embodiments, provided herein is an isolated antibody that binds to PD1 and comprises a heavy chain variable region (VH) and a light chain variable region (VL), wherein the VH CDR1 comprises the amino acid sequence of SEQ ID NO: 9, 35, or 36, the VH CDR2 comprises the amino acid sequence of SEQ ID NO: 38 or 39, the VH CDR3 comprises the amino acid sequence of SEQ ID NO: 21, the VL CDR1 comprises the amino acid sequence of SEQ ID NO: 12, the VL CDR2 comprises the amino acid sequence of SEQ ID NO: 40, and the VL CDR3 comprises the amino acid sequence of SEQ ID NO: 41.

In some embodiments, provided herein is an isolated fusion protein comprising a human interleukin 12 (IL-12) variant and an anti-PD1 antibody, wherein the fusion protein comprises the polypeptides of SEQ ID NOs: 5, 25, 6, and 4.

In some embodiments, provided herein is an isolated fusion protein comprising a human interleukin 12 (IL-12) variant and an anti-PD1 antibody, wherein the fusion protein comprises the polypeptides of SEQ ID NOs: 15, 26, 16, and 4.

In some embodiments, provided herein is an isolated polynucleotide or polynucleotides comprising one or more nucleotide sequences of encoding a human interleukin 12 (IL-12) variant comprising an amino acid substitution at position Y167 of SEQ ID NO: 1 (IL-12 p35 subunit) and position D93 of SEQ ID NO: 2 (IL-12 p40 subunit), wherein the one or more nucleotide sequences comprise a nucleotide sequence of SEQ ID NO: 44 and a nucleotide sequence of SEQ ID NO: 45.

In some embodiments, provided herein is an isolated polynucleotide or polynucleotides comprising one or more nucleotide sequences encoding the VH, VL, or both of an antibody that binds PD1, wherein the polynucleotide(s) comprise the VH nucleic acid sequence of SEQ ID NO: 46, the VL nucleic acid sequence of SEQ ID NO: 47, or both the VH nucleic acid sequence of SEQ ID NO: 46 and the VL nucleic acid sequence of SEQ ID NO: 47.

In some embodiments, provided herein is an isolated polynucleotide or polynucleotides comprising one or more nucleotide sequences encoding any one or more of the heavy chain, light chain. IL-12 p40 subunit, or heavy chain-IL12 p35 fusion polypeptide of a fusion protein comprising a human IL-12 variant and an anti-PD1 antibody, wherein the polynucleotide(s) comprise the heavy chain nucleic acid sequence of SEQ ID NO: 48, the light chain nucleic acid sequence of SEQ ID NO: 50, the IL-12 p40 subunit nucleic acid sequence of SEQ ID NO: 45, the heavy chain-IL12 p35 fusion polypeptide nucleic acid sequence of SEQ ID NO: 49 or each of the heavy chain nucleic acid sequence of SEQ ID NO: 48, the light chain nucleic acid sequence of SEQ ID NO: 50, the IL-12 p40 subunit nucleic acid sequence of SEQ ID NO: 45, and the heavy chain-IL12 p35 fusion polypeptide nucleic acid sequence of SEQ ID NO: 49.

In some embodiments, provided herein is an isolated polynucleotide or polynucleotides comprising one or more nucleotide sequences encoding any one or more of the heavy chain, light chain. IL-12 p40 subunit, or heavy chain-IL12 p35 fusion polypeptide of a fusion protein comprising a human IL-12 variant and an anti-PD1 antibody, wherein the polynucleotide(s) comprise the heavy chain encoding nucleic acid sequence of the insert of the plasmid deposited with the ATCC and having ATCC Accession Number PTA-127517, the light chain encoding nucleic acid sequence of the insert of the plasmid deposited with the ATCC and having ATCC Accession Number PTA-127519, the IL-12 p40 subunit encoding nucleic acid sequence of the insert of the plasmid deposited with the ATCC and having ATCC Accession Number PTA-127520, the heavy chain-IL12 p35 fusion polypeptide encoding nucleic acid sequence of the insert of the plasmid deposited with the ATCC and having ATCC Accession Number PTA-127518, or each of the heavy chain encoding nucleic acid sequence of the insert of the plasmid deposited with the ATCC and having ATCC Accession Number PTA-127517, the light chain encoding nucleic acid sequence of the insert of the plasmid deposited with the ATCC and having ATCC Accession Number PTA-127519, the IL-12 p40 subunit encoding nucleic acid sequence of the insert of the plasmid deposited with the ATCC and having ATCC Accession Number PTA-127520, and the heavy chain-IL12 p35 fusion polypeptide encoding nucleic acid sequence of the insert of the plasmid deposited with the ATCC and having ATCC Accession Number PTA-127518.

### BRIEF DESCRIPTION OF THE DRAWINGS

FIG. 1A and FIG. 1B show results from assays comparing the ability of the antibodies GBT-PD1-0013 [referred to as PD1(NB) in FIGS. 1A and 1B] and a known blocking anti-human PD1 antibody [referred to as PD1(B) in FIGS. 1A and 1B] to simultaneously bind to PD1. In both FIG. 1A and FIG. 1B the X axis shows the antibody(s) incubated with PD1-expressing cells. In FIG. 1A the Y axis shows the % of PD1-expressing cells that are bound by GBT-PD1-0013/PD1(NB). In FIG. 1B the Y axis shows the % of the PD1-expressing cells that are bound by PD1(B).

FIG. 2 shows a schematic of the structure an IL-12 mutein/anti-PD1 fusion protein as provided herein.

FIG. 3 shows the result of an assay comparing the activities of various mouse surrogate IL-12 mutein/anti-PD1 fusion proteins. The X-axis shows the concentration of IL-12 mutein/anti-PD1 fusion proteins (ligand) in microgram/ml, and the Y-axis shows the concentration of interferon gamma (IFNg) in picogram/ml.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Exemplary embodiments (E) of the invention provided herein include:
E1. An isolated human interleukin 12 (IL-12) variant comprising an amino acid substitution at position Y167 of SEQ ID NO: 1 (IL-12 p35 subunit) and position D93 of SEQ ID NO: 2 (IL-12 p40 subunit).
E2. The IL-12 variant of E1, wherein the Y167 substitution is Y167A
E3. The IL-12 variant of any one of E1-E2, wherein the D93 substitution is D93L
E4. The IL-12 variant of any one of E1-E3, wherein the Y167 substitution is Y167A and the D93 substitution is D93L.
E5. The IL-12 variant of any one of E1-E4, wherein the p40 subunit further comprises one or more mutations to reduce the binding of IL-12 to heparin.
E6. The IL-12 variant of E5, wherein the mutations to reduce the binding of IL-12 to heparin comprise the substitutions K258G, S259G, and K260G, and deletions of R261, E262, K263, and K264 of SEQ ID NO: 2.
E7. An isolated human interleukin 12 (IL-12) variant comprising the amino acid substitutions Y167A of SEQ ID NO: 1 (IL-12 p35 subunit) and D93L of SEQ ID NO: 2 (IL-12 p40 subunit).
E8. The IL-12 variant of E7, wherein the p40 subunit further comprises one or more mutations to reduce the binding of IL-12 to heparin.
E9. The IL-12 variant of E8, wherein the mutations to reduce the binding of IL-12 to heparin comprise the substitutions K258G, S259G, and K260G, and deletions of R261, E262, K263, and K264 of SEQ ID NO: 2.
E10. An isolated human interleukin 12 (IL-12) variant comprising one or both of i) a polypeptide comprising the amino acid sequence of SEQ ID NO: 3 (variant IL-12 p35 subunit) and ii) a polypeptide comprising the amino acid sequence of SEQ ID NO: 4 (variant IL-12 p40 subunit).
E11. The IL-12 variant of E10, wherein the IL-12 variant comprises i) a polypeptide comprising the amino acid sequence of SEQ ID NO: 3 and ii) a polypeptide comprising the amino acid sequence of SEQ ID NO: 4.
E12. An isolated human interleukin 12 (IL-12) variant comprising an amino acid substitution at one or more of positions: F39 of SEQ ID NO: 1 (IL-12 p35 subunit), 152 of SEQ ID NO: 1, Y167 of SEQ ID NO: 1, K85 of SEQ ID NO: 2 (IL-12 p40 subunit) and D93 of SEQ ID NO: 2.
E13. The IL-12 variant of E12, wherein the F39 substitution is F39R or F39A.
E14. The IL-12 variant of any one of E12-E13, wherein the I52 substitution is I52E, I52R, or I52H
E15. The IL-12 variant of any one of E12-E14, wherein the Y167 substitution is Y167A.
E16. The IL-12 variant of any one of E12-E15, wherein the K85 substitution is K85E.
E17. The IL-12 variant of any one of E12-E16, wherein the D93 substitution is D93L.
E18. The IL-12 variant of any one of E12-E17, wherein the p40 subunit further comprises one or more mutations to reduce the binding of IL-12 to heparin.
E19. The IL-12 variant of E18, wherein the mutations to reduce the binding of IL-12 to heparin comprise the substitutions K258G. S259G, and K260G, and deletions of R261, E262, K263, and K264 of SEQ ID NO: 2.
E20. The IL-12 variant of any one of E1-E19, wherein the IL-12 variant has one or both of i) reduced binding to the human IL-12 receptor as compared to wild-type human IL-12 to the human IL-12 receptor and ii) reduced activity as compared to wild-type human IL-12 to the human IL-12 receptor.
E21. An isolated antibody that binds to PD1 and comprises a heavy chain variable region (VH) and a light chain variable region (VL), wherein the VH CDR1 comprises the amino acid sequence of SEQ ID NOs: 9, 35, or 36, the VH CDR2 comprises the amino acid sequence of SEQ ID NO: 10 or 37, the VH CDR3 comprises the amino acid sequence of SEQ ID NO. 11, the VL CDR1 comprises the amino acid sequence of SEQ ID NO: 12, the VL CDR2 comprises the amino acid sequence of SEQ ID NO: 13, and the VL CDR3 comprises the amino acid sequence of SEQ ID NO: 14.
E22. The antibody of E21, wherein the VH comprises the amino acid sequence of SEQ ID NO: 7 or a variant of SEQ ID NO: 7 comprising one to four amino acid substitutions at residues that are not within a CDR, and the VL comprises the amino acid sequence of SEQ ID NO: 8 or a variant of SEQ ID NO: 8 comprising one to four amino acid substitutions at residues that are not within a CDR.
E23. The antibody of E22, wherein the VH comprises the amino acid sequence of SEQ ID NO: 7 and the VL comprises the amino acid sequence of SEQ ID NO: 8.
E24. An isolated antibody that binds to PD1 comprising: a VH amino acid sequence comprising a VH CDR1, VH CDR2, and VH CDR3 of the amino acid sequence of SEQ ID NO: 7 and a VL CDR1, VL CDR2, and VL CDR3 of the amino acid sequence of SEQ ID NO: 8.
E25. An isolated antibody comprising a heavy chain comprising the amino acid sequence of SEQ ID NO: 5, 51 or 52 and a light chain comprising the amino acid sequence of SEQ ID NO: 6, wherein the C-terminal lysine of SEQ ID NO: 5, 51, or 52 is optional.
E26. An isolated antibody that binds to PD1 and comprises a heavy chain variable region (VH) and a light chain variable region (VL), wherein the VH CDR1 comprises the amino acid sequence of SEQ ID NO: 19, 35, or 36, the VH CDR2 comprises the amino acid sequence of SEQ ID NO: 20 or 37, the VH CDR3 comprises the amino acid sequence of SEQ ID NO. 21, the VL CDR1 comprises the amino acid sequence of SEQ ID NO: 22, the VL CDR2 comprises the amino acid sequence of SEQ ID NO. 23, and the VL CDR3 comprises the amino acid sequence of SEQ ID NO: 24.
E27. The antibody of E26, wherein the VH comprises the amino acid sequence of SEQ ID NO: 17 or a variant of SEQ ID NO: 17 comprising one to four amino acid substitutions at residues that are not within a CDR, and the VL comprises the amino acid sequence of SEQ ID NO: 18 or a variant of SEQ ID NO: 18 comprising one to four amino acid substitutions at residues that are not within a CDR.
E28. The antibody of E27, wherein the VH comprises the amino acid sequence of SEQ ID NO: 17 and the VL comprises the amino acid sequence of SEQ ID NO: 18.
E29. An isolated antibody that binds to PD1 comprising: a VH amino acid sequence comprising a VH CDR1, VH CDR2, and VH CDR3 of the amino acid sequence of SEQ ID NO: 17 and a VL CDR1, VL CDR2, and VL CDR3 of the amino acid sequence of SEQ ID NO: 18.
E30. An isolated antibody comprising a heavy chain comprising the amino acid sequence of SEQ ID NO: 15, 53, or 54 and a light chain comprising the amino acid sequence of SEQ ID NO: 16, wherein the C-terminal lysine of SEQ ID NO: 15, 53, or 54 is optional.
E31. An isolated antibody that binds to PD1 and comprises a heavy chain variable region (VH) and a light chain variable region (VL), wherein the VH CDR1 comprises the amino acid sequence of SEQ ID NO: 9, 35, or 36, the VH CDR2 comprises the amino acid sequence of SEQ ID NO: 38 or 39, the VH CDR3 comprises the amino acid sequence of SEQ ID NO: 21, the VL CDR1 comprises the amino acid sequence of SEQ ID NO: 12, the VL CDR2 comprises the amino acid sequence of SEQ ID NO: 40, and the VL CDR3 comprises the amino acid sequence of SEQ ID NO: 41.
E32. The antibody of E31, wherein the VH comprises the amino acid sequence of SEQ ID NO: 33 and the VL comprises the amino acid sequence of SEQ ID NO: 34.
E33. The antibody of any one of E21-E32, wherein the antibody does not block the binding of PDL1 to PD1.
E34. The antibody of any one of E21-E33, wherein the antibody does not block the binding to PD1 of an anti-PD1 antibody that inhibits the interaction between PD1 and PDL1.
E35. The antibody of any one of E21-E34, wherein the antibody has modifications in the Fc domain to reduce binding to the Fc gamma receptor.
E36. An isolated fusion protein comprising a human interleukin 12 (IL-12) variant of any one of E1-E20 linked to an anti-PD1 antibody.
E37. The fusion protein of E36, wherein the anti-PD1 antibody is the antibody of any one of E21-E34.
E38. The fusion protein of any one of E36-E37, wherein the IL-12 variant comprises an amino acid substitution at position Y167 of SEQ ID NO: 1 (IL-12 p35 subunit) and position D93 of SEQ ID NO: 2 (IL-12 p40 subunit).
E39. The fusion protein of E38, wherein the Y167 substitution is Y167A and the D93 substitution is D93L.
E40. The fusion protein of any one of E36-E39, wherein the IL-12 variant comprises one of both of i) a polypeptide comprising the amino acid sequence of SEQ ID NO: 3 (variant IL-12 p35 subunit) and ii) a polypeptide comprising the amino acid sequence of SEQ ID NO: 4 (IL-12 p40 subunit).
E41. The fusion protein of any one of E36-E40, wherein the IL-12 variant comprises i) a polypeptide comprising the amino acid sequence of SEQ ID NO: 3 and ii) a polypeptide comprising the amino acid sequence of SEQ ID NO: 4.
E42. The fusion protein of any one of E36-E41, wherein the antibody comprises a heavy chain variable region (VH) and a light chain variable region (VL), wherein the VH comprises the amino acid sequence of SEQ ID NO: 7 and the VL comprises the amino acid sequence of SEQ ID NO: 8.
E43. The fusion protein of any one of E36-E41, wherein the antibody comprises a heavy chain variable region (VH) and a light chain variable region (VL), wherein the VH comprises the amino acid sequence of SEQ ID NO: 17 and the VL comprises the amino acid sequence of SEQ ID NO: 18.
E44. An isolated fusion protein comprising a human interleukin 12 (IL-12) variant and an anti-PD1 antibody, wherein the fusion protein comprises the polypeptides of SEQ ID NOs: 5, 25, 6, and 4.
E45. An isolated fusion protein comprising a human interleukin 12 (IL-12) variant and an anti-PD1 antibody, wherein the fusion protein comprises the polypeptides of SEQ ID NOs: 15, 26, 16, and 4.
E46. An isolated polynucleotide or polynucleotides comprising one or more nucleotide sequences encoding one or more of the IL-12 variants, anti-PD1 antibodies, fusion proteins or a polypeptide thereof of any one of E1-E45.
E47. An isolated polynucleotide or polynucleotides comprising one or more nucleotide sequences of encoding a human interleukin 12 (IL-12) variant comprising an amino acid substitution at position Y167 of SEQ ID NO: 1 (IL-12 p35 subunit) and position D93 of SEQ ID NO: 2 (IL-12 p40 subunit), wherein the one or more nucleotide sequences comprise a nucleotide sequence of SEQ ID NO: 44 and a nucleotide sequence of SEQ ID NO: 45.
E48. An isolated polynucleotide or polynucleotides comprising one or more nucleotide sequences encoding the VH, VL, or both of an antibody that binds PD1, wherein the polynucleotide(s) comprise the VH nucleic acid sequence of SEQ ID NO: 46, the VIL nucleic acid sequence of SEQ ID NO: 47, or both the VH nucleic acid sequence of SEQ ID NO: 46 and the VL nucleic acid sequence of SEQ ID NO: 47.
E49. An isolated polynucleotide or polynucleotides comprising one or more nucleotide sequences encoding any one or more of the heavy chain, light chain, IL-12 p40 subunit, or heavy chain-IL12 p35 fusion polypeptide of a fusion protein comprising a human IL-12 variant and an anti-PD1 antibody, wherein the polynucleotide(s) comprise the heavy chain nucleic acid sequence of SEQ ID NO: 48, the light chain nucleic acid sequence of SEQ ID NO: 50, the IL-12 p40 subunit nucleic acid sequence of SEQ ID NO: 45, the heavy chain-IL12 p35 fusion polypeptide nucleic acid sequence of SEQ ID NO: 49 or each of the heavy chain nucleic acid sequence of SEQ ID NO: 48, the light chain nucleic acid sequence of SEQ ID NO: 50, the IL-12 p40 subunit nucleic acid sequence of SEQ ID NO: 45, and the heavy chain-IL12 p35 fusion polypeptide nucleic acid sequence of SEQ ID NO: 49.
E50. An isolated polynucleotide or polynucleotides comprising one or more nucleotide sequences encoding any one or more of the heavy chain, light chain, IL-12 p40 subunit, or heavy chain-IL12 p35 fusion polypeptide of a fusion protein comprising a human IL-12 variant and an anti-PD1 antibody, wherein the polynucleotide(s) comprise the heavy chain encoding nucleic acid sequence of the insert of the plasmid deposited with the ATCC and having ATCC Accession Number PTA-127517, the light chain encoding nucleic acid sequence of the insert of the plasmid deposited with the ATCC and having ATCC Accession Number PTA-127519, the IL-12 p40 subunit encoding nucleic acid sequence of the insert of the plasmid deposited with the ATCC and having ATCC Accession Number PTA-127520, the heavy chain-IL12 p35 fusion polypeptide encoding nucleic acid sequence of the insert of the plasmid deposited with the ATCC and having ATCC Accession Number PTA-127518, or each of the heavy chain encoding nucleic acid sequence of the insert of the plasmid deposited with the ATCC and having ATCC Accession Number PTA-127517, the light chain encoding nucleic acid sequence of the insert of the plasmid deposited with the ATCC and having ATCC Accession Number PTA-127519, the IL-12 p40 subunit encoding nucleic acid sequence of the insert of the plasmid deposited with the ATCC and having ATCC Accession Number PTA-127520, and the heavy chain-IL12 p35 fusion polypeptide encoding nucleic acid sequence of the insert of the plasmid deposited with the ATCC and having ATCC Accession Number PTA-127518.
E51. The polynucleotide or polynucleotides of any one of E46-E50, wherein said polynucleotide(s) is RNA or DNA.
E52. The polynucleotide or polynucleotides of any one of E46-E51, wherein said polynucleotide(s) comprises at least one chemical modification.
E53. The polynucleotide or polynucleotides of E52, wherein the chemical modification wherein is selected from pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine,), 5-methoxyuridine and 2′-O-methyl uridine.
E54. The polynucleotide or polynucleotides of any one of E46-E53, wherein said polynucleotide does not comprise a chemical modification.
E55. A vector comprising the polynucleotide or polynucleotides of any one of E46-E54.
E56. An isolated host cell comprising the polynucleotide or polynucleotides of any one of E46-E54 or the vector of E55.
E57. A method of producing an IL-12 variant, anti-PD1 antibody, or fusion protein comprising culturing the host cell of E56 under conditions that result production of the IL-12 variant, anti-PD1 antibody, or fusion protein, and optionally further recovering the IL-12 variant, anti-PD1 antibody, or fusion protein.
E58. A pharmaceutical composition comprising the IL-12 variant, anti-PD1 antibody, or fusion protein of any one of E1-E45 and a pharmaceutically acceptable carrier.
E59. A method of treating cancer in a subject, the method comprising administering to the subject in need thereof a therapeutically effective amount of the pharmaceutical composition of E58 or the IL-12 variant, anti-PD1 antibody, or fusion protein of any one of E1-E45.
E60. The IL-12 variant, anti-PD1 antibody, or fusion protein of any one of E1-E45 for use as a medicament, optionally for use a medicament for cancer.
E61. The IL-12 variant, anti-PD1 antibody, or fusion protein of any one of E1-E45 for use for the treatment of cancer.
E62. The IL-12 variant, anti-PD1 antibody, fusion protein, or method of any of E59-E61 wherein the cancer is bladder cancer, breast cancer, clear cell kidney cancer, head/neck squamous cell carcinoma [squamous cell carcinoma of the head and neck (SCCHN)], lung squamous cell carcinoma, lung adenocarcinoma, malignant melanoma, non-small-cell lung cancer (NSCLC), ovarian cancer, pancreatic cancer, prostate cancer, renal cell carcinoma (RCC), small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), endometrial cancer, B-cell acute lymphoblastic leukemia, colorectal cancer (CRC), glioblastoma, uterine cancer, cervical cancer, penile cancer, gastric cancer (GC), non-melanoma skin cancer, NSCLC that has been previously treated with platinum-based treatment and/or a checkpoint inhibitor (e.g. a PD(L)1 inhibitor), RCC that has been previously treated with a tyrosine kinase inhibitor and/or a checkpoint inhibitor (e.g. a PD(L)1 inhibitor), ovarian cancer, microsatellite stable (MSS) CRC, hepatocellular carcinoma (HCC), or bladder cancer.
E63. The IL-12 variant, anti-PD1 antibody, fusion protein, or method of any of E59-E62, wherein the cancer was previously treated with a PD(L)1 inhibitor which is different than the anti-PD1 antibody of any one of E21-E35.
E64. The IL-12 variant, anti-PD1 antibody, fusion protein, or method of any of E59-E62, wherein the cancer is treated in combination with a PD(L)1 inhibitor which is different than the anti-PD1 antibody of any one of E21-E35.

The section headings used herein are for organizational purposes only and are not to be construed as limiting the subject matter described.

All references cited herein, including patent applications, patent publications, UniProtKB accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd, edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000): Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of IL-12 variant or fusion protein) means that the parameter may vary by as much as 10% below or above the stated numerical value for that parameter. For example, a dose of about 5 mg means 5%±10%, i.e. it may vary between 4.5 mg and 5.5 mg.

An “antibody” refers to an immunoglobulin molecule capable of specific binding to a target, such as a polypeptide, carbohydrate, polynucleotide, lipid, etc., through at least one antigen binding site, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding site.

An antibody includes an antibody of any class, such as IgG, IgA, or IgM (or sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The extended definition is the combination of the Kabat and Chothia definitions. The AbM definition uses an integrated suite of computer programs produced by Oxford Molecular Group that model antibody structure. See, e.g., Martin et al., 1989, Proc Natl Acad Sci (USA), 86:9268-9272; “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999. “Ab Initio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions.

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

A ‘Fc domain’ refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ (or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

A “bispecific antibody” refers to a molecule that has binding specificity for at least two different epitopes. In some embodiments, bispecific antibodies can bind simultaneously two different antigens. In other embodiments, the two different epitopes may reside on the same antigen.

The term “half maximal effective concentration (EC₅₀)” refers to the concentration of a therapeutic agent which causes a response halfway between the baseline and maximum after a specified exposure time. The therapeutic agent may cause inhibition or stimulation. The EC₅₀ value is commonly used, and is used herein, as a measure of potency.

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, or reduces a biological activity or effect of another molecule, such as a receptor. The term antagonist encompasses substances (such as an antibody) which bind to a molecule to prevent or reduce the activity of that molecule.

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE system). For example, SPR can be used in an in vitro competitive binding inhibition assay to determine the ability of one antibody to inhibit the binding of a second antibody. Another assay for measuring antibody competition uses an ELISA-based approach.

An “Fc receptor” (FcR) refers to a receptor that binds to the Fc domain of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward, Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

A “fragment” or “portion” of an antibody or polypeptide may be made by truncation, e.g., by removal of one or more amino acids from the amino terminal end, the carboxy terminal end or both ends of a polypeptide. One, 2, 3, 4, 5, 6, 7, 8, 9, 10, up to 20, up to 30 up to 40, up to 50, up to 60, up to 70, up to 80 up to 100 or more amino acids may be removed from the amino terminal end, the carboxy terminal end or both ends of the polypeptide to produce a fragment or portion. A fragment or portion may be made by one or more deletions of amino acids from the polypeptide. A fragment or portion may be made by one or more deletions of amino acids from the polypeptide as well as removal of one or more amino acids from the amino terminal end, the carboxy terminal end or both ends of a polypeptide.

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc domain amino acid sequences and increased or decreased ADCC activity are described, e.g., in U.S. Pat. Nos. 7,923,538, and 7,994,290.

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc domain.

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine. Exemplary potential conservative substitutions include the following amino acid pairs that may be substituted: Ala/Val; Arg/Lys; Asn/Gln; Asp/Glu; Cys/Ser; Gln/Asn; Glu/Asp; Gly/Ala; His/Arg; Ile/Leu; Met/Leu; Phe/Tyr; Pro/Ala; Ser/Thr; Trp/Tyr; Val/Leu.

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm.

To determine percent identity, sequences can be aligned using the methods and computer programs, including BLAST, available over the world wide web at the National Center for Biotechnology Information (NCBI). Other alignment programs include MegAlign® program in the Lasergene® suite of bioinformatics software (DNASTAR®, Inc., Madison, WI). Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. Smith-Waterman is one type of algorithm that permits gaps in sequence alignments. See Meth. Mal. Biol. 70: 173-187 (1997). Also, the GAP program using the Needleman and Wunsch alignment method can be utilized to align sequences. See J. Mal. Biol. 48: 443-453 (1970).

Also, of interest is the BestFit program using the local homology algorithm of Smith and Waterman (1981. Advances in Applied Mathematics 2: 482-489) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from Genetics Computing Group (GCG) package, from Madison, WI, USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient” includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder, or condition is cancer.

The terms “prevent” or “prevention” refer to preventing a disease, condition or disorder in an individual that may be predisposed to the disease, condition or disorder but does not yet experience or display the pathology or symptomatology of the disease. In some embodiments, prevention is assessed on a population basis such that an agent is considered to “prevent” a particular disease, disorder or condition if a statistically significant decrease in the development, frequency or intensity of the disease, disorder or condition is observed in a population susceptible to the disease, disorder or condition. Prevention may be considered complete when onset of disease, disorder or condition has been delayed for a predefined period of time.

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development. In some embodiments, a subject is a patient with cancer.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### IL-12 Variants

In some embodiments, provided herein are interleukin 12 (IL-12) variants. IL-12 variants are also known as IL-12 “muteins”.

IL-12 is a heterodimer that contains two subunits, p35 (also known as IL-12alpha; it is encoded by the IL-12A gene) and p40 (also known as IL-12beta; it is encoded by the IL-12B gene). The two IL-12 subunits can form an inter-subunit disulfide bond between C177 of p40 and C74 of p35.

The amino acid sequence of the mature wild-type human IL-12 p35 subunit is herein provided as SEQ ID NO: 1:

(SEQ ID NO: 1)
RNLPVATPDPGMFPCLHHSQNLLRAVSNMLQKARQTLEFYPCTSEEIDH
EDITKDKTSTVEACLPLELTKNESCLNSRETSFITNGSCLASRKTSFMM
ALCLSSIYEDLKMYQVEFKTMNAKLLMDPKRQIFLDQNMLAVIDELMQA
LNFNSETVPQKSSLEEPDFYKTKIKLCILLHAFRIRAVTIDRVMSYLNA
S.

The mature human p35 subunit (SEQ ID NO: 1) is generated from a full-length p35 polypeptide that also includes a 22-amino acid signal peptide which is cleaved during intracellular processing of the initially translated precursor protein. The full-length human p35 amino acid sequence including the signal peptide is available under UniProt Accession Number P29459, is herein provided as SEQ ID NO: 28 (the signal peptide is underlined):

(SEQ ID NO: 28)
MCPARSLLLVATLVLLDHLSLARNLPVATPDPGMFPCLHHSQNLLRAVS
NMLQKARQTLEFYPCTSEEIDHEDITKDKTSTVEACLPLELTKNESCLN
SRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLC
ILLHAFRIRAVTIDRVMSYLNAS

All references herein to a specific amino acid number in the IL-12p35 amino acid sequence are to the position of the amino acid in the mature IL-12p35 sequence lacking the signal peptide (not to the position of the amino acid in the precursor, full-length protein). For example, amino acid “R1” in the IL-12p35 amino acid sequence refers to the arginine (R) in the first position in SEQ ID NO: 1.

The amino acid sequence of the mature wild-type human IL-12 p40 subunit is herein provided as SEQ ID NO: 2:

(SEQ ID NO: 2)
IWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVT
CGAATLSAERVRGDNKEYEYSVECQEDSACPAAEESLPIEVMVDAVHKL
KYENYTSSFFIRDIIKPDPPKNLQLKPLKNSRQVEVSWEYPDTWSTPHS
YFSLTFCVQVQGKSKREKKDRVFTDKTSATVICRKNASISVRAQDRYYS
SSWSEWASVPCS.

The mature human p40 subunit (SEQ ID NO: 2) is generated from a full-length p40 polypeptide that also includes a 22-amino acid signal peptide which is cleaved during intracellular processing of the initially translated precursor protein. The full-length human p40 amino acid sequence including the signal peptide is available under UniProt Accession Number P29460, is herein provided as SEQ ID NO: 29 (the signal peptide is underlined):

(SEQ ID NO: 29)
MCHQQLVISWFSLVFLASPLVAIWELKKDVYVVELDWYPDAPGEMVVLT
CDTPEEDGITWTLDQSSEVLGSGKTLTIQVKEFGDAGQYTCHKGGEVLS
HSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQED
SACPAAEESLPIEVMVDAVHKLKYENYTSSFFIRDIIKPDPPKNLQLKP
LKNSRQVEVSWEYPDTWSTPHSYFSLTFCVQVQGKSKREKKDRVFTDKT
SATVICRKNASISVRAQDRYYSSSWSEWASVPCS

All references herein to a specific amino acid number in the IL-12p40 amino acid sequence are to the position of the amino acid in the mature IL-12p40 sequence lacking the signal peptide (not to the position of the amino acid in the precursor, full-length protein). For example, amino acid “W2” in the IL-12p40 amino acid sequence refers to the tryptophan (W) in the second position in SEQ ID NO: 2.

As used herein, an IL-12 “variant” or “mutein” refers to any IL-12 molecule that contains at least one amino acid change in at least one of the p35 or p40 subunits as compared to the amino acid sequence of the wild-type mature p35 subunit (SEQ ID NO: 1) or the wild-type mature p40 subunit (SEQ ID NO: 2). In some embodiments, an IL-12 variant provided herein may have at least one amino acid change in both the p35 and p40 subunits as compared to the amino acid sequence of wild-type mature p35 (SEQ ID NO: 1) or p40 (SEQ ID NO: 2).

In some embodiments, provided herein is an IL-12 variant, wherein the variant comprises an amino acid substitution at one or more of the following positions in SEQ ID NO: 1 (p35 subunit): F39, I52, or Y167. In some embodiments, the F39 substitution is F39R or F39A. In some embodiments, the I52 substitution is I52E, I52R, or I52H. In some embodiments, the Y167 substitution is Y167A.

In some embodiments, provided herein is an IL-12 variant, wherein the variant comprises an amino acid substitution at one or more of the following positions in SEQ ID NO: 2 (p40 subunit): K85 or D93. In some embodiments, the K85 substitution is K85E. In some embodiments, the D93 substitution is D93L.

In some embodiments, provided herein is an IL-12 variant, wherein the variant comprises an amino acid substitution at one or more of the following positions in SEQ ID NO: 1 (p35 subunit): F39, I52, or Y167, and at one or more of the following positions in SEQ ID NO: 2 (p40 subunit): K85 or D93. In some embodiments, the F39 substitution is F39R or F39A. In some embodiments, the I52 substitution is I52E, I52R, or I52H. In some embodiments, the Y167 substitution is Y167A. In some embodiments, the K85 substitution is K85E. In some embodiments, the D93 substitution is D93L.

In some embodiments, provided herein is an IL-12 variant having reduced activity, wherein the IL-12 variant is the variant referred to as H1, H2, H3, H4, H5, H6, H7, H8, H9, H10, H11, H12, H13, H14, H15, H16, H17, H18, H19, H20, H21, H22, H23, H24, H25, H30, H31, or H32 in Example 1 Table 10, and wherein the respective variant has the respective mutations in one or both of the p35 and p40 subunits as shown in Table 10.

In some embodiments, IL-12 variants provided herein have reduced activity. As used herein “reduced activity” of IL-12 variants refers to reduced activity as compared to activity of the corresponding wild-type IL-12 (e.g. wild-type human IL-12). “Activity” of IL-12 may be assessed by any suitable assay known in the art to measure IL-12 activity. For example, IL-12 activity may be assessed by measuring STAT4 phosphorylation (pSTAT4) in cells in response to IL-12 exposure. STAT4 phosphorylation is an early downstream effect of IL-12-induced receptor dimerization, and thus it can serve as a receptor proximal readout for IL-12 activity. In another example, IL-12 activity may be assessed by examining T helper type 1 (“Th1”)-related gene transcription, such as IFN gamma gene transcription. pSTAT4 leads to upregulation of Th1-related gene transcription and thus IFN gamma (or other Th1-related genes) can serve as a downstream readout for IL-12 activity. In another example, IL-12 activity may be indirectly assessed by measuring the affinity of an IL-12 variant for the IL-12 receptor.

IL-12 variants that have reduced activity may also be described as being “potency reduced” IL-12 variants, “partial agonists”, or the like.

In some embodiments, IL-12 variants provided herein have reduced binding to the IL-12 receptor as compared to the binding of wild-type IL-12 to the IL-12 receptor. The IL-12 receptor is a heterodimer containing the subunits IL-12Rbeta1 (see UniProt ID NO: P42701 for human IL-12Rbeta1 details) and IL-12Rbeta2 (see UniProt ID NO: Q99665 for human IL-12Rbeta2 details). IL-12 variants having reduced binding to the IL-12 receptor may be useful, for example, in circumstances in which the IL-12 variant still binds to the IL-12 receptor with sufficient affinity in order to activate the receptor in certain circumstances, but which provides less activation of the IL-12 receptor as compared to wild-type IL-12. Although IL-12 activity may be useful therapeutically in order to activate a subject's immune system, excessive IL-12 activity may be harmful to patients in the event of overstimulation of the immune response, which can result in treatment-related adverse events (TRAEs), such as cytokine release syndrome (CRS).

In some embodiments, an IL-12 variant provided herein has activity that is less than 90%, less than 80%, less than 70%, less than 60%, less than 50%, less than 40%, less than 30%, less than 20%, less than 10%, less than 9%, less than 8%, less than 7%, less than 6%, less than 5%, less than 4%, less than 3%, less than 2%, less than 1%, less than 0.5%, less than 0.1%, or less than 0.01% of the activity of the same amount of the corresponding wild-type IL-12 molecule when tested under the same experimental conditions.

In some embodiments, an IL-12 variant provided herein has activity that is reduced by 10% or more, 20% or more, 30% or more, 40% or more, 50% or more, 60% or more, 70% or more, 80% or more, 90% or more, 95% or more, 96% or more, 97% or more, 98% or more, 99% or more, 99.9% or more, 99.99% or more as compared the activity of the same amount of the corresponding wild-type IL-12 molecule when tested under the same experimental conditions.

In some embodiments, an IL-12 variant provided herein has activity that is reduced by 2-fold or more, 3-fold or more, 5-fold or more, 10-fold or more, 20-fold or more, 50-fold or more, 100-fold or more, 500-fold or more, 1000-fold or more, 5000-fold or more, 10000-fold or more; 15000-fold or more, 20000-fold or more, 23000-fold or more, 25000-fold or more, 50000-fold or more, or 100,000-fold or more as compared to the same amount of the corresponding wild-type IL-12 molecule when tested under the same experimental conditions.

In some embodiments, the affinity of an IL-12 variant provided herein for the IL-12 receptor is less than 90%, less than 80%, less than 70%, less than 60%, less than 50%, less than 40%, less than 30%, less than 20%, less than 10%, less than 9%, less than 8%, less than 7%, less than 6%, less than 5%, less than 4%, less than 3%, less than 2%, less than 1%, less than 0.5%, or less than 0.1% of the affinity the corresponding wild-type IL-12 molecule for the IL-12 receptor when tested under the same experimental conditions.

In some embodiments, the affinity of an IL-12 variant provided herein for the IL-12 receptor is reduced by 10% or more, 20% or more, 30% or more, 40% or more, 50% or more, 60% or more, 70% or more, 80% or more, 90% or more, 95% or more, 96% or more, 97% or more, 98% or more, 99% or more, 99.9% or more, 99.99% or more as compared to the affinity of the corresponding wild-type IL-12 molecule for the IL-12 receptor when tested under the same experimental conditions.

In some embodiments, the affinity of an IL-12 variant provided herein for the IL-12 receptor is reduced by 2-fold or more, 3-fold or more, 5-fold or more, 10-fold or more, 20-fold or more, 50-fold or more, 100-fold or more, 500-fold or more, 1000-fold or more, 5000-fold or more, 10000-fold or more, 15000-fold or more, 20000-fold or more, 23000-fold or more, 25000-fold or more, 50000-fold or more, or 100.000-fold or more as compared to the affinity of the corresponding wild-type IL-12 molecule for the IL-12 receptor when tested under the same experimental conditions.

Exemplary IL-12 variants provided herein include those shown in Example 1 and described in the claims and enumerated embodiments. IL-12 variants include, for example, the H10 mutein which comprises the p35 amino acid sequence of SEQ ID NO: 3 and the p40 amino acid sequence of SEQ ID NO: 4, as shown in Table 1.

TABLE 1

| ID | Sequence |
|---|---|
| IL-12 H10 | RNLPVATPDPGMFPCLHHSQNLLRAVSNMLQKARQTLEFYPCTSEEIDHEDITK |
| variant p35 | DKTSTVEACLPLELTKNESCLNSRETSFITNGSCLASRKTSFMMALCLSSIYEDLK |
| subunit | MYQVEFKTMNAKLLMDPKRQIFLDQNMLAVIDELMQALNFNSETVPQKSSLEEP |
| | DFAKTKIKLCILLHAFRIRAVTIDRVMSYLNAS (SEQ ID NO: 3) |
| IL-12 H10 | IWELKKDVYVVELDWYPDAPGEMVVLTCDTPEEDGITWTLDQSSEVLGSGKTLT |
| variant p40 | IQVKEFGDAGQYTCHKGGEVLSHSLLLLHKKEDGIWSTLILKDQKEPKNKTFLRC |
| subunit | EAKNYSGRFTCWWLTTISTDLTFSVKSSRGSSDPQGVTCGAATLSAERVRGDN |
| | KEYEYSVECQEDSACPAAEESLPIEVMVDAVHKLKYENYTSSFFIRDIIKPDPPKN |
| | LQLKPLKNSRQVEVSWEYPDTWSTPHSYFSLTFCVQVQGGGGDRVFTDKTSAT |
| | VICRKNASISVRAQDRYYSSSWSEWASVPCS (SEQ ID NO: 4) |

In some embodiments, provided herein is an IL-12 variant, wherein the variant comprises an amino acid substitution at one or more of the following positions in SEQ ID NO: 1 (p35 subunit): F39, I52, or Y167, wherein the variant comprises an amino acid sequence having 80% or greater, 85% or greater, 90% or greater, 95% or greater, 96% or greater, 97% or greater, 98% or greater, or 99% or greater sequence identity to the amino acid sequence of SEQ ID NO: 1. In some embodiments, the F39 substitution is F39R or F39A. In some embodiments, the I52 substitution is I52E, I52R, or I52H. In some embodiments, the Y167 substitution is Y167A.

In some embodiments, provided herein is an IL-12 variant, wherein the variant comprises an amino acid substitution at one or more of the following positions in SEQ ID NO: 2 (p40 subunit). K85 or D93, wherein the variant comprises an amino acid sequence having 80% or greater, 85% or greater, 90% or greater, 95% or greater, 96% or greater, 97% or greater, 98% or greater, or 99% or greater sequence identity to the amino acid sequence of SEQ ID NO: 2. In some embodiments, the K85 substitution is K85E. In some embodiments, the D93 substitution is D93L.

In some embodiments, provided herein is an IL-12 variant, wherein the variant comprises an amino acid substitution at one or more of the following positions in SEQ ID NO: 1 (p35 subunit): F39, I52, or Y167, and at one or more of the following positions in SEQ ID NO: 2 (p40 subunit): K85 or D93, wherein the variant comprises an amino acid sequence having 80% or greater, 85% or greater, 90% or greater, 95% or greater, 96% or greater, 97% or greater, 98% or greater, or 99% or greater sequence identity to the amino acid sequence of SEQ ID NO: 1 and an amino acid sequence having 80% or greater, 85% or greater, 90% or greater, 95% or greater, 96% or greater, 97% or greater, 98% or greater, or 99% or greater sequence identity to the amino acid sequence of SEQ ID NO: 2. In some embodiments, the F39 substitution is F39R or F39A. In some embodiments, the I52 substitution is I52E, I52R, or I52H. In some embodiments, the Y167 substitution is Y167A. In some embodiments, the K85 substitution is K85E. In some embodiments, the D93 substitution is D93L.

In some embodiments, provided herein is an IL-12 variant, wherein the variant comprises the amino acid substitution Y167A in SEQ ID NO: 1 (p35 subunit) and the amino acid substitution D93L in SEQ ID NO: 2 (p40 subunit), wherein the variant comprises an amino acid sequence having 80% or greater, 85% or greater, 90% or greater, 95% or greater, 96% or greater, 97% or greater, 98% or greater, or 99% or greater sequence identity to the amino acid sequence of SEQ ID NO: 1 and an amino acid sequence having 80% or greater, 85% or greater, 90% or greater, 95% or greater, 96% or greater, 97% or greater, 98% or greater, or 99% or greater sequence identity to the amino acid sequence of SEQ ID NO: 2.

In some embodiments, provided herein is an IL-12 variant provided herein linked to another protein, such as an antibody. These molecules are referred to as “IL-12 variant fusion proteins” are described in detail later herein.

### Antibodies to PD1

The present disclosure also provides antibodies that bind to human PD1. PD1 (programmed cell death protein 1; also known as PD-1 and CD279) is an immune checkpoint protein. PD1 is a type 1 transmembrane receptor that was originally identified in a T cell line undergoing activation-induced apoptosis. PD1 is expressed on various immune cells such as T cells, B cells, and macrophages. The ligands for PD1 are the B7 family members PD-L1 (B7-H1) and PD-L2 (B7-DC). PD1 down-regulates immune cell activity; thus inhibition of PD1 results in increased immune cell activity, such as increased T cell proliferation and activation, increased IFN, IL-2 and TNF secretion from immune cells, and increased immune cell anti-tumor response.

As used herein, the term “PD1” includes variants, isoforms, homologs, orthologs and paralogs of PD1. In some embodiments, an antibody disclosed herein cross-reacts with PD1 from species other than human, such as PD1 of cynomolgus monkey, as well as different forms of PD1. In some embodiments, an antibody may be completely specific for human PD1 and may not exhibit species cross-reactivity (e.g., does not bind mouse PD1) or other types of cross-reactivity (e.g., does not bind other receptors in the tumor necrosis factor receptor family). As used herein the term PD1 refers to naturally occurring human PD1 unless contextually dictated otherwise. Therefore, a “PD1 antibody” “anti-PD1 antibody” or other similar designation means any antibody (as defined herein) that binds or reacts with PD1, an isoform, fragment or derivative thereof.

There are multiple different anti-PD1 antibodies that have been developed for the treatment of cancer, such nivolumab and pembrolizumab. Typically, these antibodies provide a therapeutic effect by reducing PD1 biological activity, such as by inhibiting the binding of PD1 to its ligands PDL1 and PDL2. Anti-PD1 antibodies that inhibit the binding of PD1 to one or both of PDL1 and PDL2 are referred to herein as “blocking”, “inhibitory”, or “antagonist” anti-PD1 antibodies.

In contrast, in some embodiments, provided herein are antibodies that bind to PD1 but that do not inhibit (or do not inhibit completely) the binding of PDL1 and PDL2 to PD1. These antibodies can bind to PD1 at the same time that PD1 is bound to PDL1 or PDL2. These antibodies are referred to herein as “non-blocking” anti-PD1 antibodies. Non-blocking anti-PD1 antibodies are useful for their ability to bind PD1 (even if they do not inhibit PD1 activity that is mediated by the PD1-PDL1/PDL2 interaction). For example, non-blocking anti-PD1 antibodies can be used to target molecules that are linked to the non-blocking anti-PD1 antibody to PD1-expressing cells, such as T cells.

In some embodiments, a non-blocking anti-PD1 antibody provided herein can bind to PD1 at the same time that PD1 is bound by a blocking anti-PD1 antibody

As used herein, the term “PD1-binding” antibody refers to both blocking and non-blocking anti-PD1 antibodies.

In some embodiments, an anti-PD1 antibody of the disclosure encompasses an antibody that one or both of i) competes for binding to human PD1 with or ii) binds the same epitope as, an antibody comprising the amino acid sequence of a heavy chain variable region set forth as SEQ ID NO: 7 and the amino acid sequence of a light chain variable region set forth as SEQ ID NO: 8. In some embodiments, an anti-PD1 antibody of the disclosure encompasses an antibody that one or both of i) competes for binding to human PD1 with or ii) binds the same epitope as, an antibody comprising the amino acid sequence of a heavy chain variable region set forth as SEQ ID NO: 17 and the amino acid sequence of a light chain variable region set forth as SEQ ID NO: 18. In some embodiments, an anti-PD1 antibody of the disclosure encompasses an antibody that one or both of i) competes for binding to human PD1 with or ii) binds the same epitope as, an antibody comprising the amino acid sequence of a heavy chain variable region set forth as SEQ ID NO: 33 and the amino acid sequence of a light chain variable region set forth as SEQ ID NO: 34.

Anti-PD1 antibodies of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies) In some embodiments, an anti-PD1 antibody is a monoclonal antibody. In some embodiments, an anti-PD1 antibody is a human or humanized antibody. In some embodiments, an anti-PD1 antibody is a chimeric antibody. In some embodiments, an anti-PD1 antibody provided herein is IgG1 subclass. In some embodiments, an anti-PD1 antibody provided herein has knob-in-hole mutations in Fc chains to promote heterodimerization between the chains. In some embodiments, an anti-PD1 antibody provided herein has mutations in the Fc chains to decrease binding affinity to a human Fc gamma receptor.

In some embodiments, the invention provides an antibody having a light chain variable region (VL) sequence and a heavy chain variable region (VH) sequence as found in Table 2 or variants thereof. In Table 2, the underlined sequences are CDR sequences (Kabat definition) and the sequences in bold are CDR sequences (Chothia definition).

TABLE 2

| ID | Sequence |
|---|---|
| VH | EVQLVESGGGLVQPGGSLRLSCAASGFSFGDFDMRWFRQAPGKGLEWVGTIKSRA |
| TPP- | YLEATEFAASVEGRFTISRDDAKNSAYLQMNSLRAEDTAVYYCTRDAYSSGLLDYW |
| 77658 | GQGTLVTVSS (SEQ ID NO: 7) |
| VL | DIQMTQSPSSLSASVGDRVTITCRASQGISNYLAWFQQKPGKAPKRLIYAAQIPGSG |
| TPP- | VPSRFSGSGSGTDFTLTISSLQPEDFATYYCLQHYSYPLTFGGGTKVEIK (SEQ ID |
| 77658 | NO: 8) |
| VH | EVQLVESGGGLVQPGGSLRLSCAASGFSFGDFDMRWFRQAPGKGLEWVGLIKSRA |
| TPP- | YLEATEFAASVEGRFTISRDDAKNSAYLQMNSLRAEDTAVYYCTRDSYSSGLLDYW |
| 76868 | GQGTLVTVSS (SEQ ID NO: 17) |
| VL | DIQMTQSPSSLSASVGDRVTITCRASQGISNYLAWFQQKPGKAPKRLIYAAQIPGSG |
| TPP- | VPSRFSGSGSGTDFTLTISSLQPEDFATYYCLQHYSYPLTFGGGTKVEIK (SEQ ID |
| 76868 | NO: 18) |
| VH | EVQLVESGGGLVQPGGSLRLSCAASGFSFGDFDMRWFRQAPGKGLEWVGLIKSKA |
| TPP- | YRYATEFAASVEGRFTISRDDAKNSAYLQMNSLRAEDTAVYYCTRDSYSSGLLDYW |
| 68807 | GQGTLVTVSS (SEQ ID NO: 33) |
| VL | DIQMTQSPSSLSASVGDRVTITCRASQGISNYLAWFQQKPGKAPKRLIYAASIPGSGV |
| TPP- | PSRFSGSGSGTDFTLTISSLQPEDFATYYCLQHKSYPLTFGGGTKVEIK (SEQ ID |
| 68807 | NO: 34) |

In some embodiments, provided herein is an antibody comprising a VH as set forth in SEQ ID NO: 7 and a VL as set forth in SEQ ID NO: 8. In some embodiments, provided herein is an antibody comprising a VH as set forth in SEQ ID NO: 17 and a VL as set forth in SEQ ID NO: 18. In some embodiments, provided herein is an antibody comprising a VH as set forth in SEQ ID NO: 33 and a VL as set forth in SEQ ID NO: 34.

The invention also provides CDR portions of antibodies to PD1. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the antibody comprises three CDRs of a heavy chain variable region shown in Table 2. In some embodiments, the antibody comprises three CDRs of a light chain variable region shown in Table 2. In some embodiments, the antibody comprises three CDRs of a heavy chain variable region shown in Table 2, and three CDRs of a light chain variable region shown in Table 2.

Table 3 provides examples of CDR sequences of anti-PD1 antibodies provided herein. CDRs that are not notated with any specific CDR definition in Table 3 have the same CDR definition according to Chothia, Kabat, and Extended definitions.

TABLE 3
Anti-PD1 antibodies (mAbs) and their antigen-binding CDR sequences

| mAb | CDR1 | CDR2 | CDR3 |
|---|---|---|---|
| VH TPP-77658 | GFSFGDF (SEQ ID | KSRAYLEA (SEQ ID NO: | DAYSSGLLDY |
| | NO: 35) (Chothia) | 37) (Chothia) | (SEQ ID NO: 11) |
| | DFDMR (SEQ ID | TIKSRAYLEATEFAASVEG | |
| | NO: 36) (Kabat) | (SEQ ID NO: 10) (Kabat | |
| | GFSFGDFDMR | and Extended) | |
| | (SEQ ID NO: 9) | | |
| | (Extended) | | |
| VL TPP-77658 | RASQGISNYLA | AAQIPGS (SEQ ID NO: | LQHYSYPLT (SEQ |
| | (SEQ ID NO: 12) | 13) | ID NO: 14) |
| VH TPP-76868 | GFSFGDF (Chothia) | KSRAYLEA (Chothia) | DSYSSGLLDY |
| | (SEQ ID NO: 35) | (SEQ ID NO: 37) | (SEQ ID NO: 21) |
| | DFDMR (Kabat) | LIKSRAYLEATEFAASVEG | |
| | (SEQ ID NO: 36) | (Kabat and Extended) | |
| | GFSFGDFDMR | (SEQ ID NO: 20) | |
| | (Extended) (SEQ ID | | |
| | NO: 19) | | |
| VL TPP-76868 | RASQGISNYLA | AAQIPGS (SEQ ID NO: | LQHYSYPLT (SEQ |
| | (SEQ ID NO: 22) | 23) | ID NO: 24) |
| VH TPP-68807 | GFSFGDF (SEQ ID | KSKAYRYA (SEQ ID NO: | DSYSSGLLDY |
| | NO: 35) (Chothia) | 38) (Chothia) | (SEQ ID NO: 21) |
| | DFDMR (SEQ ID | LIKSKAYRYATEFAASVEG | |
| | NO: 36) (Kabat) | (SEQ ID NO: 39) (Kabat | |
| | GFSFGDFDMR | and Extended) | |
| | (SEQ ID NO: 9) | | |
| | (Extended) | | |
| VL TPP-68807 | RASQGISNYLA | AASIPGS (SEQ ID NO: 40) | LQHKSYPLT (SEQ |
| | (SEQ ID NO: 12) | | ID NO: 41) |

In some embodiments, an anti-PD1 antibody provided herein comprises three light chain CDRs and three heavy chain CDRs from an antibody as shown in Table 3.

In some embodiments, an anti-PD1 antibody comprises one or both of i) the full-length heavy chain, with or without the C-terminal lysine, or ii) the full-length light chain. The amino acid sequences of the full-length heavy chain and light chain for exemplary anti-PD1 antibodies provided herein are shown below in Table 4.

TABLE 4
Heavy chain and light chain sequences for anti-PD1 mAbs

| ID | Sequence |
|---|---|
| TPP- | EVQLVESGGGLVQPGGSLRLSCAASGFSFGDFDMRWFRQAPGKGLEWVGTIKS |
| 77658 | RAYLEATEFAASVEGRFTISRDDAKNSAYLQMNSLRAEDTAVYYCTRDAYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCVVVDVSH |
| | EDPEVKFNWYVDGVEVHNAKTKPREEQYNSTYRVVSVLTVLHQDWLNGKEYKC |
| | KVSNKALPAPIEKTISKAKGQPREPQVYTLPPCREEMTKNQVSLSCAVKGFYPSDI |
| | AVEWESNGQPENNYKTTPPVLDSDGSFFLVSKLTVDKSRWQQGNVFSCSVMHE |
| | ALHNHYTQKSLSLSPGK (SEQ ID NO: 5) |
| TPP- | EVQLVESGGGLVQPGGSLRLSCAASGFSFGDFDMRWFRQAPGKGLEWVGTIKS |
| 77658 | RAYLEATEFAASVEGRFTISRDDAKNSAYLQMNSLRAEDTAVYYCTRDAYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| without | KVEPKSCDKTHTCPPCPAPEAAGAPSVFLFPPKPKDTLMISRTPEVTCVVVDVSH |
| hole | EDPEVKFNWYVDGVEVHNAKTKPREEQYNSTYRVVSVLTVLHQDWLNGKEYKC |
| mutations | KVSNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSVMHE |
| | ALHNHYTQKSLSLSPGK (SEQ ID NO: 51) |
| TPP- | EVQLVESGGGLVQPGGSLRLSCAASGFSFGDFDMRWFRQAPGKGLEWVGTIKS |
| 77658 | RAYLEATEFAASVEGRFTISRDDAKNSAYLQMNSLRAEDTAVYYCTRDAYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| without | KVEPKSCDKTHTCPPCPAPELLGGPSVFLFPPKPKDTLMISRTPEVTCVVVDVSH |
| hole or | EDPEVKFNWYVDGVEVHNAKTKPREEQYNSTYRVVSVLTVLHQDWLNGKEYKC |
| effector | KVSNKALPAPIEKTISKAKGQPREPQVYTLPPSREEMTKNQVSLTCLVKGFYPSDI |
| null | AVEWESNGQPENNYKTTPPVLDSDGSFFLYSKLTVDKSRWQQGNVFSCSVMHE |
| mutations | ALHNHYTQKSLSLSPGK (SEQ ID NO: 52) |
| TPP- | DIQMTQSPSSLSASVGDRVTITCRASQGISNYLAWFQQKPGKAPKRLIYAAQIPGS |
| 77658 | GVPSRFSGSGSGTDFTLTISSLQPEDFATYYCLQHYSYPLTFGGGTKVEIKRTVAA |
| Light | PSVFIFPPSDEQLKSGTASVVCLLNNFYPREAKVQWKVDNALQSGNSQESVTEQ |
| Chain | DSKDSTYSLSSTLTLSKADYEKHKVYACEVTHQGLSSPVTKSFNRGEC (SEQ ID |
| | NO: 6) |
| TPP- | EVQLVESGGGLVQPGGSLRLSCAASGFSFGDFDMRWFRQAPGKGLEWVGLIKS |
| 76868 | RAYLEATEFAASVEGRFTISRDDAKNSAYLQMNSLRAEDTAVYYCTRDSYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCVVVDVSH |
| | EDPEVKFNWYVDGVEVHNAKTKPREEQYNSTYRVVSVLTVLHQDWLNGKEYKC |
| | KVSNKALPAPIEKTISKAKGQPREPQVYTLPPCREEMTKNQVSLSCAVKGFYPSDI |
| | AVEWESNGQPENNYKTTPPVLDSDGSFFLVSKLTVDKSRWQQGNVFSCSVMHE |
| | ALHNHYTQKSLSLSPGK (SEQ ID NO: 15) |
| TPP- | EVQLVESGGGLVQPGGSLRLSCAASGFSFGDFDMRWFRQAPGKGLEWVGLIKS |
| 76868 | RAYLEATEFAASVEGRFTISRDDAKNSAYLQMNSLRAEDTAVYYCTRDSYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| without | KVEPKSCDKTHTCPPCPAPEAAGAPSVFLFPPKPKDTLMISRTPEVTCVVVDVSH |
| hole | EDPEVKFNWYVDGVEVHNAKTKPREEQYNSTYRVVSVLTVLHQDWLNGKEYKC |
| mutations | KVSNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSVMHE |
| | ALHNHYTQKSLSLSPGK (SEQ ID NO: 53) |
| TPP- | EVQLVESGGGLVQPGGSLRLSCAASGFSFGDFDMRWFRQAPGKGLEWVGLIKS |
| 76868 | RAYLEATEFAASVEGRFTISRDDAKNSAYLQMNSLRAEDTAVYYCTRDSYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| without | KVEPKSCDKTHTCPPCPAPELLGGPSVFLFPPKPKDTLMISRTPEVTCVVVDVSH |
| hole or | EDPEVKFNWYVDGVEVHNAKTKPREEQYNSTYRVVSVLTVLHQDWLNGKEYKC |
| effector | KVSNKALPAPIEKTISKAKGQPREPQVYTLPPSREEMTKNQVSLTCLVKGFYPSDI |
| null | AVEWESNGQPENNYKTTPPVLDSDGSFFLYSKLTVDKSRWQQGNVFSCSVMHE |
| mutations | ALHNHYTQKSLSLSPGK (SEQ ID NO: 54) |
| TPP- | DIQMTQSPSSLSASVGDRVTITCRASQGISNYLAWFQQKPGKAPKRLIYAAQIPGS |
| 76868 | GVPSRFSGSGSGTDFTLTISSLQPEDFATYYCLQHYSYPLTFGGGTKVEIKRTVAA |
| Light | PSVFIFPPSDEQLKSGTASVVCLLNNFYPREAKVQWKVDNALQSGNSQESVTEQ |
| Chain | DSKDSTYSLSSTLTLSKADYEKHKVYACEVTHQGLSSPVTKSFNRGEC (SEQ ID |
| | NO: 16) |
| TPP- | EVQLVESGGGLVQPGGSLRLSCAASGFSFGDFDMRWFRQAPGKGLEWVGLIKS |
| 68807 | KAYRYATEFAASVEGRFTISRDDAKNSAYLQMNSLRAEDTAVYYCTRDSYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCVVVDVSH |
| | EDPEVKFNWYVDGVEVHNAKTKPREEQYNSTYRVVSVLTVLHQDWLNGKEYKC |
| | KVSNKALPAPIEKTISKAKGQPREPQVYTLPPCREEMTKNQVSLSCAVKGFYPSDI |
| | AVEWESNGQPENNYKTTPPVLDSDGSFFLVSKLTVDKSRWQQGNVFSCSVMHE |
| | ALHNHYTQKSLSLSPGK (SEQ ID NO: 42) |
| TPP- | EVQLVESGGGLVQPGGSLRLSCAASGFSFGDFDMRWFRQAPGKGLEWVGLIKS |
| 68807 | KAYRYATEFAASVEGRFTISRDDAKNSAYLQMNSLRAEDTAVYYCTRDSYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| without | KVEPKSCDKTHTCPPCPAPEAAGAPSVFLFPPKPKDTLMISRTPEVTCVVVDVSH |
| hole | EDPEVKFNWYVDGVEVHNAKTKPREEQYNSTYRVVSVLTVLHQDWLNGKEYKC |
| mutations | KVSNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSVMHE |
| | ALHNHYTQKSLSLSPGK (SEQ ID NO: 55) |
| TPP- | EVQLVESGGGLVQPGGSLRLSCAASGFSFGDFDMRWFRQAPGKGLEWVGLIKS |
| 68807 | KAYRYATEFAASVEGRFTISRDDAKNSAYLQMNSLRAEDTAVYYCTRDSYSSGLL |
| Heavy | DYWGQGTLVTVSSASTKGPSVFPLAPSSKSTSGGTAALGCLVKDYFPEPVTVSW |
| Chain | NSGALTSGVHTFPAVLQSSGLYSLSSVVTVPSSSLGTQTYICNVNHKPSNTKVDK |
| without | KVEPKSCDKTHTCPPCPAPELLGGPSVFLFPPKPKDTLMISRTPEVTCVVVDVSH |
| hole or | EDPEVKFNWYVDGVEVHNAKTKPREEQYNSTYRVVSVLTVLHQDWLNGKEYKC |
| effector | KVSNKALPAPIEKTISKAKGQPREPQVYTLPPSREEMTKNQVSLTCLVKGFYPSDI |
| null | AVEWESNGQPENNYKTTPPVLDSDGSFFLYSKLTVDKSRWQQGNVFSCSVMHE |
| mutations | ALHNHYTQKSLSLSPGK (SEQ ID NO: 56) |
| TPP- | DIQMTQSPSSLSASVGDRVTITCRASQGISNYLAWFQQKPGKAPKRLIYAASIPGS |
| 68807 | GVPSRFSGSGSGTDFTLTISSLQPEDFATYYCLQHKSYPLTFGGGTKVEIKRTVAA |
| Light | PSVFIFPPSDEQLKSGTASVVCLLNNFYPREAKVQWKVDNALQSGNSQESVTEQ |
| Chain | DSKDSTYSLSSTLTLSKADYEKHKVYACEVTHQGLSSPVTKSFNRGEC (SEQ ID |
| | NO: 43) |

In Table 5, the amino acid sequence of the “TPP-77658 Heavy Chain” (SEQ ID NO: 5) includes the following annotated features in the Fc: effector null mutations L234A, L235A, and G237A (EU numbering: underlined), and mutations to form a “hole” for a Knob-in-Hole structure: S354C, T366S, L368A, and Y407V (EU numbering; underlined). The amino acid sequence of “TPP-77658 Heavy Chain without hole mutations” (SEQ ID NO: 51) is the same as SEQ ID NO: 5 except it does not contain the mutations to form a hole; it still contains the effector null mutations L234A, L235A, and G237A (underlined). The amino acid sequence of “TPP-77658 Heavy Chain without hole or effector null mutations” (SEQ ID NO: 52) is the same as SEQ ID NO: 5 except it does not contain the hole or effector null mutations of SEQ ID NO: 5; instead it contains the corresponding wild-type amino acids.

In Table 5, the amino acid sequence of the “TPP-76868 Heavy Chain” (SEQ ID NO: 15) includes the following annotated features in the Fc: effector null mutations L234A, L235A, and G237A (EU numbering; underlined), and mutations to form a “hole” for a Knob-in-Hole structure: S354C, T366S, L368A, and Y407V (EU numbering; underlined). The amino acid sequence of “TPP-76868 Heavy Chain without hole mutations” (SEQ ID NO: 53) is the same as SEQ ID NO: 15 except it does not contain the mutations to form a hole; it still contains the effector null mutations L234A, L235A, and G237A (underlined). The amino acid sequence of “TPP-76868 Heavy Chain without hole or effector null mutations” (SEQ ID NO: 54) is the same as SEQ ID NO: 15 except it does not contain the hole or effector null mutations of SEQ ID NO: 15; instead it contains the corresponding wild-type amino acids.

In Table 5, the amino acid sequence of the “TPP-68807 Heavy Chain” (SEQ ID NO: 42) includes the following annotated features in the Fc: effector null mutations L234A, L235A, and G237A (EU numbering; underlined), and mutations to form a “hole” for a Knob-in-Hole structure: S354C, T366S, L368A, and Y407V (EU numbering: underlined). The amino acid sequence of “TPP-68807 Heavy Chain without hole mutations” (SEQ ID NO: 55) is the same as SEQ ID NO: 42 except it does not contain the mutations to form a hole; it still contains the effector null mutations L234A, L235A, and G237A (underlined). The amino acid sequence of “TPP-68807 Heavy Chain without hole or effector null mutations” (SEQ ID NO: 56) is the same as SEQ ID NO: 5 except it does not contain the hole or effector null mutations of SEQ ID NO. 42; instead it contains the corresponding wild-type amino acids.

In certain embodiments, an antibody described herein comprises an Fc domain. The Fc domain can be derived from IgA (e.g., IgA₁ or IgA₂), IgG, IgE, or IgG (e.g., IgG₁, IgG₂, IgG₃, or IgG₄). In some embodiments, an anti-PD1 antibody provided herein is an IgG1 antibody.

The invention encompasses modifications to the variable regions shown in Table 2, the CDRs shown in Table 3, and heavy chain and light chain sequences shown in Table 4. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to PD1. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86 319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

For example, in some embodiments, the constant region of an antibody provided herein is modified to have decreased binding affinity to a human Fc gamma receptor. These antibodies are also referred to as “effector null” or as having an “inert Fc domain”. Such antibodies may have, for example, one or more of the mutations L234A, L235A, and G237A in the IgG1 CH2 domain to reduce or eliminate effector function (numbering according to EU nomenclature).

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997. Chem. Immunol. 65:111-128: Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-PD1 antibodies containing variations of the variable regions shown in Table 2, the CDRs shown in Table 3, or heavy chain and light chain sequences shown in Table 4, wherein such variant polypeptides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% amino acid sequence identity to any of the amino acid sequences disclosed in Table 2, 3, or 4. These amounts are not meant to be limiting and increments between the recited percentages are specifically envisioned as part of the disclosure.

In some embodiments provided herein is an anti-PD1 antibody that comprises a VH and a VL, wherein the antibody VH has an amino acid sequence encoded by a nucleic acid sequence within the nucleic acid sequence of the insert of the plasmid deposited with the ATCC having ATCC Accession No. PTA-127517 and the antibody VL has an amino acid sequence encoded by a nucleic acid sequence within the nucleic acid sequence of the insert of the plasmid deposited with the ATCC having ATCC Accession No. PTA-127519.

The invention also encompasses fusion proteins comprising one or more components of the antibodies disclosed herein. In some embodiments, a fusion protein may be made that comprises all or a portion of an anti-PD1 antibody of the invention linked to another polypeptide. In another embodiment, only the variable domains of the anti-PD1 antibody are linked to the polypeptide. In another embodiment, the VH domain of an anti-PD1 antibody is linked to a first polypeptide, while the VL domain of an anti-PD1 antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### IL-12 Variant Fusion Proteins

In some embodiments, provided herein is an IL-12 variant provided herein linked to another protein. These molecules are referred to herein as an “IL-12 variant fusion protein”.

In some embodiments, an IL-12 variant can be linked to various other types of protein, such as an antibody, another cytokine, an enzyme, or an antibody Fc domain.

IL-12 variants provided herein can be linked to another protein in any suitable manner. For example, an Il-12 variant can be covalently linked to another protein directly (e.g. such that an amino acid of IL-12 is directly covalently linked to an amino acid of the other protein) or via a polypeptide linker. In addition, IL-12 can be linked in any orientation relative to the other protein—e.g. it can be linked at the N or C terminus of the other protein; similarly either the N or C terminus of the IL-12 subunit can be linked to the other protein.

When IL-12 is linked to another protein, one or both of the IL-12 subunits can be linked to the other protein. For example, if an IL-12 variant provided herein is linked to an antibody, in some embodiments, one subunit of IL-12 (e.g. p35) can be linked to a first chain of the antibody, and the other subunit of IL-12 (e.g. p40) can be linked to a second chain of the antibody. In other embodiments, only one of the IL-12 subunits (p35 or p40) is linked to a chain of the antibody, and the other, non-linked IL-12 subunit associates in the complex via its interaction with the linked IL-12 subunit.

In some embodiments, when Il-12 is linked to another protein, Il-12 can be prepared as single polypeptide chain containing both the p35 and p40 subunits of IL-12. Combining the sequences of the p35 and p40 subunits into a single polypeptide that can assemble as an intact IL-12 molecule may be desirable in order to facilitate the expression of recombinant IL-12 variants provided herein.

In some embodiments, an IL-12 variant is linked to an antibody. Linking of an IL-12 variant to an antibody can be useful for one or more purposes, such as 1) to target the IL-12 to the location of antibody target (e.g. if the antibody binds to a cell surface receptor, linking IL-12 to that antibody can target IL-12 to cells having that cell surface receptor) and 2) if the antibody has a therapeutic effect, IL-12 activity can be coupled with the therapeutic effect of the antibody.

In some embodiments, provided herein are fusion proteins that link an IL-12 variant provided herein and an anti-PD1 antibody (referred to herein as an “IL-12 variant/anti-PD1 fusion protein”, “IL-12 variant/anti-PD1 molecule” or the like). In some embodiments, the anti-PD1 antibody is an anti-PD1 antibody as provided herein.

High expression of PD1 is predominantly seen on CD8-positive and CD4-positive tumor-infiltrating lymphocytes (TIL) and enriched in the tumor microenvironment (TME) compared to circulating T cell subsets. This anatomical localization and cellular expression profile suggests that targeting IL-12 activity towards PD1-positive (PD1+) cells could reduce systemic activity while still enabling strong anti-tumor immunity. Overall, it is hypothesized that directing IL-12 activity from the periphery towards the tumor microenvironment will enhance anti-tumor efficacy of IL-12 in both PD(L)1 antagonist naïve and PD(L)1 antagonist-resistant tumors, while reducing systemic toxicity.

PD1-positive cells (e.g. CD8 positive T cells and CD4 positive T cells) also often contain IL-12 receptors. Thus, binding of the antibody portion of an IL-12 variant/anti-PD1 fusion protein to PD1 on an immune cell containing an IL-12 receptor brings the IL-12 variant in the fusion protein in close proximity to the IL-12 receptor on the PD1-positive cell. In other words, an IL-12 variant/anti-PD1 fusion provided herein can bind to both 1) PD1 and 2) IL-12 receptor on the same cell (e.g. a T cell). This is also referred to as “cis-targeting” of the IL-12 (i.e. targeting the IL-12 variant to the same cell that is bound by the antibody which is linked to the IL-12 variant). Cis-targeting of IL-12 variants to PD1-positive cells has multiple potential benefits. First, IL-12 variants that have reduced affinity for the IL-12 receptor (e.g. IL-12 variants as provided herein) have low activity on cells that are IL-12-receptor positive but negative for PD1, in view of the low affinity of the IL-12 variants for the IL-12 receptor. Cells that are positive for the IL-12 receptor but negative for PD1 (or that have low amounts of PD1) are most commonly circulating cells/cells in the periphery (i.e. outside the tumor microenvironment). Thus, fusion proteins containing an IL-12 variant with reduced affinity for the IL-12 receptor and an anti-PD1 antibody will have minimal activity and associated potential toxicity on peripheral cells that do not express PD1. Second, IL-12 variants that have reduced affinity for the IL-12 receptor (e.g. IL-12 variants as provided herein) can still have effective activity on cells that are IL-12-receptor positive and positive for PD1, in view of binding of the IL-12 variant/anti-PD1 fusion molecule to PD1. In this circumstance, the anti-PD1 antibody bound to PD1 effectively maintains the IL-12 variant in close proximity to the IL-12 receptor such that the IL-12 variant and the IL-12 receptor still interact sufficiently strongly to generate downstream effects of IL-12 binding to the IL-12 receptor (e.g. increase CD8 T cell cytotoxicity, promote CD4 Th1 cell differentiation, inhibit regulatory T cell (Treg) function, and increase additional cytokine and chemokine expression, resulting various anti-tumor effects such as in recruitment of immune cells, inhibition of angiogenesis, and inhibition of tumor growth). Thus, it can be said that linking an anti-PD1 antibody to an IL-12 variant that has reduced affinity for the Il-12 receptor “rescues” the activity of that IL-12 variant in the sense that the IL-12 variant will have low or no activity on IL-12 receptors unless the IL-12 variant is maintained in close physical proximity to the IL-12 receptor via being linked to an anti-PD1 antibody that is bound to a PD1 molecule on a cell surface near the IL-12 receptor on the same cell surface.

In some embodiments, an IL-12 variant/anti-PD1 fusion protein is selected after optimizing the anti-PD1 binding of the antibody portion of the fusion protein and the IL-12 activity of the IL-12 variant portion of the fusion protein, in order to achieve a selected balance of potency and efficacy of the fusion protein. In some embodiments, an IL-12 variant/anti-PD1 fusion protein provided herein is designed to have one, two, or all three of the following features: 1) deliver PD1-mediated, avidity-driven IL-12 receptor stimulation preferentially to PD1-positive cells; 2) exhibit improved therapeutic index as compared to full agonist IL-12 molecules (e.g. wild-type IL-12-Fc fusion molecules) and 3) bind to an epitope on PD1 that permits simultaneous binding of a PD1 antagonist (e.g. antibody that blocks PD1 and PDL1 interaction) to PD1, such that the PD1 antagonist activity is maintained when the IL-12 variant/anti-PD1 fusion protein binds PD1.

Exemplary IL-12 variant/anti-PD1 fusion proteins include those shown in the Examples and described in the claims and embodiments herein.

In one embodiment, provided herein is an IL-12 variant/anti-PD1 fusion protein comprising the following characteristics. The anti-PD1 portion of the fusion protein is an anti-PD1 antibody containing two heavy chains and two light chains. One of the heavy chains of the anti-PD1 antibody has at the C-terminus of the chain a linker sequence which connects to the N-terminus of the IL-12 variant p35 amino acid sequence, such that it forms a single continuous polypeptide that contains the following components (in order from N-terminus to C-terminus): anti-PD heavy chain—linker sequence—IL-12 variant p35 sequence. There is a “knob” mutation in the Fc of one of the heavy chains and a “hole” mutation in the Fc of the other heavy chain, in order to promote heterodimerization of the two heavy chains. The p40 subunit of the IL-12 variant is connected to the p35 subunit via a disulfide bond.

In one embodiment, an IL-12 variant/anti-PD1 fusion protein provided herein is a fusion protein containing the IL-12 H mutein and the anti-PD antibody TPP-77658. This fusion protein is also referred to herein as the “H10658 fusion”. There are 5 total separate polypeptides in the H10658 fusion: 1) antibody heavy chain (not linked to an IL-12 polypeptide); 2) antibody heavy chain linked to p35 of the H10 IL-12 mutein; 3) antibody light chain (copy 1); 4) antibody light chain (copy 2); 5) p40 of the IL10 IL-12 mutein. Of the 5 separate polypeptides, there are 4 different polypeptide sequences (there are two copies of the antibody light chain in the fusion protein; both light chains have the same amino acid sequence). The amino acid sequences of the polypeptides in the H10658 fusion are shown below in Table 5.

TABLE 5

| Description | Sequence |
|---|---|
| Heavy chain | EVQLVESGGGLVQPGGSLRLSCAASGFSFGDFDMRWFRQAPGKGLEWVGTI |
| TPP-77658 | KSRAYLEATEFAASVEGRFTISRDDAKNSAYLQMNSLRAEDTAVYYCTRDAYS |
| | SGLLDYWGQGTLVTVSSASTKGPSVFPLAPSSKSTSGGTAALGCLVKDYFPE |
| | PVTVSWNSGALTSGVHTFPAVLQSSGLYSLSSVVTVPSSSLGTQTYICNVNHK |
| | PSNTKVDKKVEPKSCDKTHTCPPCPAPEAAGAPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPCREEMTKNQ |
| | VSLSCAVKGFYPSDIAVEWESNGQPENNYKTTPPVLDSDGSFFLVSKLTVDKS |
| | RWQQGNVFSCSVMHEALHNHYTQKSLSLSPGK (SEQ ID NO: 5) |
| Heavy chain | EVQLVESGGGLVQPGGSLRLSCAASGFSFGDFDMRWFRQAPGKGLEWVGTI |
| TPP-77658 | KSRAYLEATEFAASVEGRFTISRDDAKNSAYLQMNSLRAEDTAVYYCTRDAYS |
| fused to p35 | SGLLDYWGQGTLVTVSSASTKGPSVFPLAPSSKSTSGGTAALGCLVKDYFPE |
| of H10 | PVTVSWNSGALTSGVHTFPAVLQSSGLYSLSSVVTVPSSSLGTQTYICNVNHK |
| | PSNTKVDKKVEPKSCDKTHTCPPCPAPEAAGAPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVCTLPPSREEMTKNQ |
| | VSLWCLVKGFYPSDIAVEWESNGQPENNYKTTPPVLDSDGSFFLYSKLTVDKS |
| | RWQQGNVFSCSVMHEALHNHYTQKSLSLSPGSGGGGSGGGGSGGGGRNL |
| | PVATPDPGMFPCLHHSQNLLRAVSNMLQKARQTLEFYPCTSEEIDHEDITKDK |
| | TSTVEACLPLELTKNESCLNSRETSFITNGSCLASRKTSFMMALCLSSIYEDLK |
| | MYQVEFKTMNAKLLMDPKRQIFLDQNMLAVIDELMQALNFNSETVPQKSSLEE |
| | PDFAKTKIKLCILLHAFRIRAVTIDRVMSYLNAS (SEQ ID NO: 25) |
| Light chain | DIQMTQSPSSLSASVGDRVTITCRASQGISNYLAWFQQKPGKAPKRLIYAAQIP |
| TPP-77658 | GSGVPSRFSGSGSGTDFTLTISSLQPEDFATYYCLQHYSYPLTFGGGTKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNR |
| | GEC (SEQ ID NO: 6) |
| p40 of H10 | IWELKKDVYVVELDWYPDAPGEMVVLTCDTPEEDGITWTLDQSSEVLGSGKT |
| | LTIQVKEFGDAGQYTCHKGGEVLSHSLLLLHKKEDGIWSTLILKDQKEPKNKTF |
| | LRCEAKNYSGRFTCWWLTTISTDLTFSVKSSRGSSDPQGVTCGAATLSAERV |
| | RGDNKEYEYSVECQEDSACPAAEESLPIEVMVDAVHKLKYENYTSSFFIRDIIK |
| | PDPPKNLQLKPLKNSRQVEVSWEYPDTWSTPHSYFSLTFCVQVQGGGGDRV |
| | FTDKTSATVICRKNASISVRAQDRYYSSSWSEWASVPCS (SEQ ID NO: 4) |

The amino acid sequences of the H10658 fusion shown in Table 5 include the following annotated features. In the Heavy Chain TPP-77658 sequence (SEQ ID NO: 5), there are effector null mutations in the Fc: L234A, L235A, and G237A (EU numbering; underlined), and mutations to form a “hole” for a Knob-in-Hole structure: S354C, T366S, L368A, and Y407V (EU numbering; underlined). In the Heavy Chain TPP-77658 fused to p35 of H10 sequence (SEQ ID NO: 25), there are effector null mutations in the Fc: L234A, L235A, and G237A (EU numbering; underlined), mutations to form a “knob” for a Knob-in-Hole structure: Y349C and T366W (EU numbering: underlined), and a linker sequence [SGGGGSGGGGSGGGG (SEQ ID NO: 27)] connecting the heavy chain and p35 of H10. The C-terminal lysine of SEQ ID NO: 5 is optional.

In one embodiment, an IL-12 variant/anti-PD1 fusion protein provided herein is a fusion protein containing the IL-12 H10 mutein and the anti-PD1 antibody TPP-76868. This fusion protein is also referred to herein as the “H10868 fusion”. There are 5 total separate polypeptides in the H10868 fusion: 1) antibody heavy chain (not linked to an IL-12 polypeptide); 2) antibody heavy chain linked to p35 of the H10 IL-12 mutein; 3) antibody light chain (copy 1); 4) antibody light chain (copy 2); 5) p40 of the IL10 IL-12 mutein. Of the 5 separate polypeptides, there are 4 different polypeptide sequences (there are two copies of the antibody light chain in the fusion protein; both light chains have the same amino acid sequence). The amino acid sequences of the polypeptides in the H10868 fusion are shown below in Table 6.

TABLE 6

| Description | Sequence |
|---|---|
| Heavy chain | EVQLVESGGGLVQPGGSLRLSCAASGFSFG |
| TPP-76868 | DFDMRWFRQAPGKGLEWVGLIKSRAYLEAT |
| | EFAASVEGRFTISRDDAKNSAYLQMNSLRA |
| | EDTAVYYCTRDSYSSGLLDYWGQGTLVTVS |
| | SASTKGPSVFPLAPSSKSTSGGTAALGCLV |
| | KDYFPEPVTVSWNSGALTSGVHTFPAVLQS |
| | SGLYSLSSVVTVPSSSLGTQTYICNVNHKP |
| | SNTKVDKKVEPKSCDKTHTCPPCPAPEAAG |
| | APSVFLFPPKPKDTLMISRTPEVTCWVDVS |
| | HEDPEVKFNWYVDGVEVHNAKTKPREEQYN |
| | STYRVVSVLTVLHQDWLNGKEYKCKVSNKA |
| | LPAPIEKTISKAKGQPREPQVYTLPPCREE |
| | MTKNQVSLSCAVKGFYPSDIAVEWESNGQP |
| | ENNYKTTPPVLDSDGSFFLVSKLTVDKSRW |
| | QQGNVFSCSVMHEALHNHYTQKSLSLSPGK |
| | (SEQ ID NO: 15) |
| Heavy | EVQLVESGGGLVQPGGSLRLSCAASGFSFG |
| Chain TPP- | DFDMRWFRQAPGKGLEWVGLIKSRAYLEAT |
| 76868 fused | EFAASVEGRFTISRDDAKNSAYLQMNSLRA |
| to p35 of | EDTAVYYCTRDSYSSGLLDYWGQGTLVTVS |
| H10 | SASTKGPSVFPLAPSSKSTSGGTAALGCLV |
| | KDYFPEPVTVSWNSGALTSGVHTFPAVLQS |
| | SGLYSLSSVVTVPSSSLGTQTYICNVNHKP |
| | SNTKVDKKVEPKSCDKTHTCPPCPAPEAAG |
| | APSVFLFPPKPKDTLMISRTPEVTCVVVDV |
| | SHEDPEVKFNWYVDGVEVHNAKTKPREEQY |
| | NSTYRVVSVLTVLHQDWLNGKEYKCKVSNK |
| | ALPAPIEKTISKAKGQPREPQVCTLPPSRE |
| | EMTKNQVSLWCLVKGFYPSDIAVEWESNGQ |
| | PENNYKTTPPVLDSDGSFFLYSKLTVDKSR |
| | WQQGNVFSCSVMHEALHNHYTQKSLSLSPG |
| | SGGGGSGGGGSGGGGRNLPVATPDPGMFPC |
| | LHHSQNLLRAVSNMLQKARQTLEFYPCTSE |
| | EIDHEDITKDKTSTVEACLPLELTKNESCL |
| | NSRETSFITNGSCLASRKTSFMMALCLSSI |
| | YEDLKMYQVEFKTMNAKLLMDPKRQIFLDQ |
| | NMLAVIDELMQALNFNSETVPQKSSLEEPD |
| | FAKTKIKLCILLHAFRIRAVTIDRVMSYLN |
| | AS (SEQ ID NO: 26) |
| Light chain | DIQMTQSPSSLSASVGDRVTITCRASQGIS |
| TPP-76868 | NYLAWFQQKPGKAPKRLIYAAQIPGSGVPS |
| | RFSGSGSGTDFTLTISSLQPEDFATYYCLQ |
| | HYSYPLTFGGGTKVEIKRTVAAPSVFIFPP |
| | SDEQLKSGTASVVCLLNNFYPREAKVQWKV |
| | DNALQSGNSQESVTEQDSKDSTYSLSSTLT |
| | LSKADYEKHKVYACEVTHQGLSSPVTKSFN |
| | RGEC |
| | (SEQ ID NO: 16) |
| P40 of H10 | IWELKKDVYVVELDWYPDAPGEMVVLTCDT |
| | PEEDGITWTLDQSSEVLGSGKTLTIQVKEF |
| | GDAGQYTCHKGGEVLSHSLLLLHKKEDGIW |
| | STLILKDQKEPKNKTFLRCEAKNYSGRFTC |
| | WWLTTISTDLTFSVKSSRGSSDPQGVTCGA |
| | ATLSAERVRGDNKEYEYSVECQEDSACPAA |
| | EESLPIEVMVDAVHKLKYENYTSSFFIRDI |
| | IKPDPPKNLQLKPLKNSRQVEVSWEYPDTW |
| | STPHSYFSLTFCVQVQGGGGDRVFTDKTSA |
| | TVICRKNASISVRAQDRYYSSSWSEWASVP |
| | CS |
| | (SEQ ID NO: 4) |

The amino acid sequences of the H10868 fusion shown in Table 6 include the following annotated features. In the Heavy Chain TPP-76868 sequence (SEQ ID NO: 15), there are effector null mutations in the Fc: L234A, L235A, and G237A (EU numbering; underlined), and mutations to form a “hole” for a Knob-in-Hole structure S354C, T366S, L368A, and Y407V (EU numbering; underlined). In the Heavy Chain TPP-76868 fused to p35 of H10 sequence (SEQ ID NO: 26), there are effector null mutations in the Fc: L234A, L235A, and G237A (EU numbering; underlined), mutations to form a “knob” for a Knob-in-Hole structure: Y349C and T366W (EU numbering; underlined), and a linker sequence [SGGGGSGGGGSGGGG (SEQ ID NO: 27)] connecting the heavy chain and p35 of H10. The C-terminal lysine of SEQ ID NO: 15 is optional.

### Biological Activity of IL-12 Variant/Anti-PD1 Fusion Proteins

In addition to binding an epitope on PD1, an IL-12 variant/anti-PD1 fusion protein of the disclosure can mediate a biological activity. That is, the disclosure includes an isolated IL-12 variant/anti-PD1 fusion protein that specifically binds PD1 and mediates at least one detectable activity selected from the following:
(i) binds specifically to human PD1;
(ii) binds specifically to cynomolgus monkey P01;
(iii) inhibits tumor growth
(iv) increases STAT4 phosphorylation;
(v) increases interferon (IFN) gamma expression;

Without being bound by a particular theory, administration of an IL-12 variant/anti-PD1 fusion protein provided herein to a subject may effectively deliver IL-12 to PD1-positive cells in the tumor microenvironment (TME) [e.g. tumor-infiltrating lymphocytes (TILs)], with minimal peripheral activity in order to enhance the anti-tumor activity of TILs, and reduce the risk of systemic toxicity from IL-12. Also, PD1-positive T cells in the TME are known to be strong mediators of antitumor activity. This may enhance IL-12 anti-tumor efficacy in both PD(L)1-naïve (i.e. not previously treated with an agent that blocks the interaction between PD1 and PDL1) and PD(L)1-resistant (i.e. previously treated with an agent that blocks the interaction between PD1 and PDL1) tumors, while reducing systematic immune system activation and potential toxicity from IL-12. PD1-positive (PD1+) cells in the tumor microenvironment include, for example, CD8 positive (CD8+) T cells, CD4 positive (CD4+) T cells, and regulatory T cells (Tregs).

Similarly, in the non-tumor microenvironment (i.e. peripheral or normal tissues), there is a lower abundance of PD1-positive cells, and thus an IL-12 variant/anti-PD1 fusion protein provided herein results in minimal activity and toxicity due to the attenuation of IL-12 activity in the IL-12 variant and reduced PD1 binding due to low numbers of PD1-positive cells.

In some embodiments, binding of an IL-12 variant/anti-PD1 fusion protein to PD1 promotes the inhibition of tumor growth in PD1/PDL1 therapy resistant cancer cells [i.e. cancer cells that are resistant to treatment with one or both of PD1 and PDL1 (collectively “PD(L)1”) inhibitors].

Engagement of the IL-12R by IL-12 results in induction of STAT4 phosphorylation (pSTAT4) leading to upregulation of T helper type 1 (“Th1”)-related gene transcription, such as IFN gamma, thereby enhancing the functional capacity of T cells. Since phosphorylation of STAT4 is a principal activation step of IL-12 induced IL-12 receptor dimerization, pSTAT4 can serve as a receptor proximal readout for IL-12 activity, and IFN gamma can serve as a downstream readout for IL-12 activity.

Polynucleotides Encoding IL-12 Variants, anti-PD1 Antibodies, Fusion Proteins and Methods of Manufacture

The disclosure also provides polynucleotides encoding any of the Il-12 variants, anti-PD1 antibodies, or fusion proteins provided herein, including portions and modified versions of these molecules. Also included are methods of making any of the IL-12 variants, anti-PD1 antibodies, and fusion proteins provided herein. Polynucleotides can be made and proteins expressed by procedures known in the art.

An anti-PD1 antibody (monoclonal or polyclonal) of interest may be sequenced and the polynucleotide sequence may then be cloned into a vector for expression or propagation. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide or polynucleotide(s) comprising a sequence or sequences encoding one or both of the p35 and p40 subunits of an IL-12 variant provided herein. In some embodiments, provided herein is a polynucleotide or polynucleotide(s) comprising a sequence or sequences encoding any one or more of the polypeptides of an IL-12 variant/anti-PD1 fusion protein provided herein. In some embodiments, provided herein is a polynucleotide or polynucleotide(s) comprising a sequence or sequences encoding one or both of the heavy chain or the light chain variable regions of an anti-PD1 antibody provided herein. The polynucleotide(s) encoding the IL-12 variant, antibody, or fusion protein of interest may be maintained in a vector in a host cell and the host cell can then be expanded and frozen for future use. Vectors (including expression vectors) and host cells are further described herein.

In some embodiments, the disclosure provides a polynucleotide encoding the amino acid sequence of the p35 subunit of the IL-12 H10 variant, wherein the polynucleotide encodes the amino acid sequence SEQ ID NO: 3. In some embodiments, a polynucleotide comprising the nucleotide sequence of SEQ ID NO: 44 encodes the amino acid sequence of SEQ ID NO: 3. The nucleotide sequence of SEQ ID NO: 44 is below in Table 7.

In some embodiments, the disclosure provides a polynucleotide encoding the amino acid sequence of the p40 subunit of the IL-12 H10 variant, wherein the polynucleotide encodes the amino acid sequence SEQ ID NO: 4. In some embodiments, a polynucleotide comprising the nucleotide sequence of SEQ ID NO: 45 encodes the amino acid sequence of SEQ ID NO: 4. The nucleotide sequence of SEQ ID NO: 45 is below in Table 7.

In some embodiments, the disclosure provides a polynucleotide encoding the amino acid sequence of the VH of the anti-PD1 mAb TPP-77658, wherein the polynucleotide encodes the amino acid sequence SEQ ID NO: 7. In some embodiments, a polynucleotide comprising the nucleotide sequence of SEQ ID NO: 46 encodes the amino acid sequence of SEQ ID NO: 7. The nucleotide sequence of SEQ ID NO: 46 is below in Table 7.

In some embodiments, the disclosure provides a polynucleotide encoding the amino acid sequence of the VL of the anti-PD1 mAb TPP-77658, wherein the polynucleotide encodes the amino acid sequence SEQ ID NO: 8. In some embodiments, a polynucleotide comprising the nucleotide sequence of SEQ ID NO: 47 encodes the amino acid sequence of SEQ ID NO 8. The nucleotide sequence of SEQ ID NO: 47 is below in Table 7.

In some embodiments, the disclosure provides a polynucleotide encoding the amino acid sequence of the TPP-77658 heavy chain of the H10658 fusion, wherein the polynucleotide encodes the amino acid sequence SEQ ID NO: 5. In some embodiments, a polynucleotide comprising the nucleotide sequence of SEQ ID NO. 48 encodes the amino acid sequence of SEQ ID NO: 5. The nucleotide sequence of SEQ ID NO: 48 is below in Table 7.

In some embodiments, the disclosure provides a polynucleotide encoding the amino acid sequence of the TPP-77658 heavy chain-H10 mutein p35 fusion of the H10658 fusion, wherein the polynucleotide encodes the amino acid sequence SEQ ID NO: 25. In some embodiments, a polynucleotide comprising the nucleotide sequence of SEQ ID NO: 49 encodes the amino acid sequence of SEQ ID NO: 25. The nucleotide sequence of SEQ ID NO: 49 is below in Table 7.

In some embodiments, the disclosure provides a polynucleotide encoding the amino acid sequence of the TPP-77658 light chain of the H10658 fusion, wherein the polynucleotide encodes the amino acid sequence SEQ ID NO: 6. In some embodiments, a polynucleotide comprising the nucleotide sequence of SEQ ID NO: 50 encodes the amino acid sequence of SEQ ID NO: 6. The nucleotide sequence of SEQ ID NO: 50 is below in Table 7.

In some embodiments, the disclosure provides a polynucleotide encoding the amino acid sequence of the H10 mutein p40 subunit of the H10658 fusion, wherein the polynucleotide encodes the amino acid sequence SEQ ID NO: 4. In some embodiments, a polynucleotide comprising the nucleotide sequence of SEQ ID NO: 45 encodes the amino acid sequence of SEQ ID NO: 4. The nucleotide sequence of SEQ ID NO: 45 is below in Table 7.

In some embodiments, the disclosure provides a polynucleotide encoding the amino acid sequence of the p35 subunit of the IL-12 H10 variant, wherein the polynucleotide comprises the nucleotide sequence of SEQ ID NO: 44.

In some embodiments, the disclosure provides a polynucleotide encoding the amino acid sequence of the p40 subunit of the IL-12 H10 variant, wherein the polynucleotide comprises the nucleotide sequence of SEQ ID NO: 45.

In some embodiments, the disclosure provides a polynucleotide encoding the amino acid sequence of the VH of the anti-PD1 mAb TPP-77658, wherein the polynucleotide comprises the nucleotide sequence of SEQ ID NO: 46.

In some embodiments, the disclosure provides a polynucleotide encoding the amino acid sequence of the VL of the anti-PD1 mAb TPP-77658, wherein the polynucleotide comprises the nucleotide sequence of SEQ ID NO: 47.

In some embodiments, the disclosure provides a polynucleotide encoding the amino acid sequence of the TPP-77658 heavy chain of the H10658 fusion, wherein the polynucleotide comprises the nucleotide sequence of SEQ ID NO: 48.

In some embodiments, the disclosure provides a polynucleotide encoding the amino acid sequence of the TPP-77658 heavy chain-H10 mutein p35 fusion of the H10658 fusion, wherein the polynucleotide comprises the nucleotide sequence of SEQ ID NO: 49.

In some embodiments, the disclosure provides a polynucleotide encoding the amino acid sequence of the TPP-77658 light chain of the H10658 fusion, wherein the polynucleotide comprises the nucleotide sequence of SEQ ID NO: 50.

In some embodiments, the disclosure provides a polynucleotide encoding the amino acid sequence of the H10 mutein p40 subunit of the H10658 fusion, wherein the polynucleotide comprises the nucleotide sequence of SEQ ID NO: 45.

TABLE 7
Nucleotide sequences:

| Description | Sequence |
|---|---|
| Nucleotide | CGGAACCTCCCCGTCGCAACTCCTGACCCGGGGATGTTCCCTTGTCTGCACCATAGCC |
| sequence | AGAACCTGTTGAGAGCCGTGTCCAACATGCTCCAGAAAGCCCGGCAGACTCTTGAGTT |
| encoding p35 | TTACCCATGCACCTCCGAAGAAATCGACCATGAAGATATTACCAAAGACAAGACCTCCA |
| of IL-12 | CTGTGGAAGCGTGCTTGCCACTTGAGCTGACTAAGAACGAAAGCTGCCTGAACTCCCG |
| variant H10 | GGAGACTTCTTTCATCACCAATGGTTCCTGCCTTGCGTCCCGCAAGACCTCTTTTATGAT |
| (SEQ ID NO: | GGCCCTGTGCCTGTCAAGCATATACGAAGATCTGAAGATGTACCAAGTCGAGTTCAAGA |
| 3) | CCATGAATGCGAAGCTCCTTATGGACCCTAAGCGGCAGATCTTCCTGGATCAGAACATG |
| | CTGGCTGTGATCGACGAGCTGATGCAGGCTCTGAACTTCAACAGCGAGACAGTGCCGC |
| | AAAAGTCCAGCCTGGAAGAACCCGACTTCGCCAAGACCAAGATCAAGCTGTGCATTCT |
| | GCTGCACGCATTCAGGATCAGGGCAGTCACTATTGACAGAGTGATGTCCTACCTGAAC |
| | GCCAGC (SEQ ID NO: 44) |
| Nucleotide | ATCTGGGAACTGAAGAAAGATGTGTACGTGGTCGAACTTGACTGGTACCCCGATGCGC |
| sequence | CTGGAGAGATGGTCGTGCTGACTTGCGATACGCCTGAGGAAGATGGAATCACTTGGAC |
| encoding p40 | TCTCGACCAGTCGTCCGAAGTCCTCGGGTCGGGAAAGACCCTGACAATCCAGGTCAAG |
| of IL-12 | GAGTTCGGGGACGCCGGACAGTACACTTGCCACAAGGGCGGAGAAGTGCTGTCACAC |
| variant H10; | TCCCTGCTGCTCCTCCACAAGAAGGAAGATGGCATCTGGTCCACTCTGATCCTCAAGGA |
| also p40 of | CCAGAAGGAGCCGAAGAACAAGACTTTCCTGCGCTGCGAGGCCAAGAACTACTCCGGA |
| H10658 | CGGTTCACGTGTTGGTGGCTGACCACCATTAGCACCGACCTGACCTTCTCCGTGAAGT |
| fusion (SEQ | CCAGCCGGGGGAGCAGCGACCCGCAGGGAGTGACCTGTGGCGCCGCGACCCTCTCC |
| ID NO: 4) | GCTGAGCGCGTGCGGGGAGACAACAAGGAATATGAGTACAGCGTGGAGTGTCAGGAA |
| | GATTCCGCCTGTCCTGCTGCCGAAGAGTCGCTGCCAATTGAAGTGATGGTCGATGCCG |
| | TGCATAAGTTGAAATACGAGAACTACACCTCGTCGTTCTTCATCCGGGACATCATTAAG |
| | CCCGACCCGCCCAAGAACTTGCAGCTGAAGCCCCTGAAGAACTCGAGACAGGTCGAAG |
| | TGTCCTGGGAGTATCCCGACACCTGGTCCACCCCCCATTCGTACTTCTCGCTGACTTTC |
| | TGTGTGCAAGTGCAGGGTGGGGGGGGGACAGGGTGTTCACCGATAAGACCTCAGCC |
| | ACTGTGATTTGCCGCAAGAACGCGTCAATTTCAGTCAGGGCCCAGGATCGGTATTACTC |
| | CTCGTCATGGTCCGAATGGGCCTCCGTGCCCTGCTCG (SEQ ID NO: 45) |
| Nucleotide | GAGGTGCAACTGGTGGAAAGCGGAGGAGGCCTGGTGCAGCCCGGCGGATCTCTGCG |
| sequence | GCTGTCTTGTGCCGCTTCTGGCTTCAGCTTCGGCGACTTCGACATGCGGTGGTTTAGA |
| encoding VH | CAGGCCCCTGGCAAGGGCCTCGAGTGGGTGGGCACCATCAAAAGCAGAGCTTATCTG |
| of TPP- | GAAGCCACCGAGTTCGCCGCCAGCGTGGAAGGCAGATTCACCATCAGCCGGGACGAC |
| 77658 (SEQ | GCCAAGAACTCCGCCTACCTGCAGATGAACAGCCTGAGAGCCGAGGACACCGCCGTG |
| ID NO: 7) | TACTACTGCACCAGAGATGCCTACAGCAGCGGCCTGCTGGATTACTGGGGCCAGGGCA |
| | CACTGGTCACAGTGTCCAGC (SEQ ID NO: 46) |
| Nucleotide | GATATCCAGATGACCCAGAGCCCTAGCTCTCTGAGCGCCAGCGTGGGCGACAGAGTGA |
| sequence | CCATCACCTGTAGAGCCTCTCAGGGCATCTCCAACTACCTGGCTTGGTTCCAGCAGAAA |
| encoding VL | CCTGGCAAGGCCCCTAAGGGGCTGATCTACGCCGCTCAGATCCCAGGCAGCGGCGTC |
| of TPP- | CCCAGCAGATTCAGCGGCTCCGGCTCTGGCACCGACTTCACCCTGACCATTAGCAGCC |
| 77658 (SEQ | TGCAACCTGAGGACTTTGCCACATATTACTGCCTGCAGCACTACAGCTACCCCCTGACA |
| ID NO: 8) | TTCGGCGGAGGAACAAAGGTGGAAATCAAG (SEQ ID NO: 47) |
| Nucleotide | GAGGTGCAACTGGTGGAAAGCGGAGGAGGCCTGGTGCAGCCCGGCGGATCTCTGCG |
| sequence | GCTGTCTTGTGCCGCTTCTGGCTTCAGCTTCGGCGACTTCGACATGCGGTGGTTTAGA |
| encoding | CAGGCCCCTGGCAAGGGCCTCGAGTGGGTGGGCACCATCAAAAGCAGAGCTTATCTG |
| TPP-77658 | GAAGCCACCGAGTTCGCCGCCAGCGTGGAAGGCAGATTCACCATCAGCCGGGACGAC |
| heavy chain | GCCAAGAACTCCGCCTACCTGCAGATGAACAGCCTGAGAGCCGAGGACACCGCCGTG |
| of H10658 | TACTACTGCACCAGAGATGCCTACAGCAGCGGCCTGCTGGATTACTGGGGCCAGGGCA |
| fusion (SEQ | CACTGGTCACAGTGTCCAGCGCGTCGACCAAGGGCCCATCGGTCTTCCCCCTGGCACC |
| ID NO: 5) | CTCCTCCAAGAGCACCTCTGGGGGCACAGCGGCCCTGGGCTGCCTGGTCAAGGACTA |
| | CTTCCCCGAACCGGTGACGGTGTCGTGGAACTCAGGCGCCCTGACCAGCGGCGTGCA |
| | CACCTTCCCGGCTGTCCTACAGTCCTCAGGACTCTACTCCCTCAGCAGCGTGGTGACC |
| | GTGCCCTCCAGCAGCTTGGGCACCCAGACCTACATCTGCAACGTGAATCACAAGCCCA |
| | GCAACACCAAGGTGGACAAGAAAGTTGAGCCCAAATCTTGTGACAAAACTCACACATGC |
| | CCACCGTGCCCAGCACCTGAAGCCGCTGGGGCACCGTCAGTCTTCCTCTTCCCCCCAA |
| | AACCCAAGGACACCCTCATGATCTCCCGGACCCCTGAGGTCACATGCGTGGTGGTGGA |
| | CGTGAGCCACGAAGACCCTGAGGTCAAGTTCAACTGGTACGTGGACGGCGTGGAGGT |
| | GCATAATGCCAAGACAAAGCCGCGGGAGGAGCAGTACAACAGCACGTACCGTGTGGTC |
| | AGCGTCCTCACCGTCCTGCACCAGGACTGGCTGAATGGCAAGGAGTACAAGTGCAAGG |
| | TCTCCAACAAAGCCCTCCCAGCCCCCATCGAGAAAACCATCTCCAAAGCCAAAGGGCA |
| | GCCCCGAGAACCACAGGTGTACACCCTGCCCCCATGCCGGGAGGAGATGACCAAGAA |
| | CCAGGTCAGCCTGTCCTGCGCGGTCAAAGGCTTCTATCCCAGCGACATCGCCGTGGAG |
| | TGGGAGAGCAATGGGCAGCCGGAGAACAACTACAAGACCACGCCTCCCGTGCTGGAC |
| | TCCGACGGCTCCTTCTTCCTCGTTAGCAAGCTCACCGTGGACAAGAGCAGGTGGCAGC |
| | AGGGGAACGTCTTCTCATGCTCCGTGATGCATGAGGCTCTGCACAACCACTACACGCA |
| | GAAGAGCCTCTCCCTGTCCCCCGGAAAA (SEQ ID NO: 48) |
| Nucleotide | GAGGTGCAACTGGTGGAAAGCGGAGGAGGCCTGGTGCAGCCCGGCGGATCTCTGCG |
| sequence | GCTGTCTTGTGCCGCTTCTGGCTTCAGCTTCGGCGACTTCGACATGCGGTGGTTTAGA |
| encoding | CAGGCCCCTGGCAAGGGCCTCGAGTGGGTGGGCACCATCAAAAGCAGAGCTTATCTG |
| TPP-77658 | GAAGCCACCGAGTTCGCCGCCAGCGTGGAAGGCAGATTCACCATCAGCCGGGACGAC |
| heavy chain- | GCCAAGAACTCCGCCTACCTGCAGATGAACAGCCTGAGAGCCGAGGACACCGCCGTG |
| H10 mutein | TACTACTGCACCAGAGATGCCTACAGCAGCGGCCTGCTGGATTACTGGGGCCAGGGCA |
| p35 fusion of | CACTGGTCACAGTGTCCAGCGCGTCGACCAAGGGCCCATCGGTCTTCCCCCTGGCACC |
| H10658 | CTCCTCCAAGAGCACCTCTGGGGGCACAGCGGCCCTGGGCTGCCTGGTCAAGGACTA |
| fusion (SEQ | CTTCCCCGAACCGGTGACGGTGTCGTGGAACTCAGGCGCCCTGACCAGCGGCGTGCA |
| ID NO: 25) | CACCTTCCCGGCTGTCCTACAGTCCTCAGGACTCTACTCCCTCAGCAGCGTGGTGACC |
| | GTGCCCTCCAGCAGCTTGGGCACCCAGACCTACATCTGCAACGTGAATCACAAGCCCA |
| | GCAACACCAAGGTGGACAAGAAAGTTGAGCCCAAATCTTGTGACAAAACTCACACATGC |
| | CCACCGTGCCCAGCACCTGAAGCCGCTGGGGCACCGTCAGTCTTCCTCTTCCCCCCAA |
| | AACCCAAGGACACCCTCATGATCTCCCGGACCCCTGAGGTCACATGCGTGGTGGTGGA |
| | CGTGAGCCACGAAGACCCTGAGGTCAAGTTCAACTGGTACGTGGACGGCGTGGAGGT |
| | GCATAATGCCAAGACAAAGCCGCGGGAGGAGCAGTACAACAGCACGTACCGTGTGGTC |
| | AGCGTCCTCACCGTCCTGCACCAGGACTGGCTGAATGGCAAGGAGTACAAGTGCAAGG |
| | TCTCCAACAAAGCCCTCCCAGCCCCCATCGAGAAAACCATCTCCAAAGCCAAAGGGCA |
| | GCCCCGAGAACCACAGGTGTGCACCCTGCCCCCATCCCGGGAGGAGATGACCAAGAA |
| | CCAGGTCAGCCTGTGGTGCCTGGTCAAAGGCTTCTATCCCAGCGACATCGCCGTGGAG |
| | TGGGAGAGCAATGGGCAGCCGGAGAACAACTACAAGACCACGCCTCCCGTGCTGGAC |
| | TCCGACGGCTCCTTCTTCCTCTATAGCAAGCTCACCGTGGACAAGAGCAGGTGGCAGC |
| | AGGGGAACGTCTTCTCATGCTCCGTGATGCATGAGGCTCTGCACAACCACTACACGCA |
| | GAAGAGCCTCTCCCTGTCCCCCGGAAGCGGTGGAGGAGGTTCCGGTGGGGGAGGTTC |
| | AGGTGGCGGAGGGCGGAACCTCCCCGTCGCAACTCCTGACCCGGGGATGTTCCCTTG |
| | TCTGCACCATAGCCAGAACCTGTTGAGAGCCGTGTCCAACATGCTCCAGAAAGCCCGG |
| | CAGACTCTTGAGTTTTACCCATGCACCTCCGAAGAAATCGACCATGAAGATATTACCAAA |
| | GACAAGACCTCCACTGTGGAAGCGTGCTTGCCACTTGAGCTGACTAAGAACGAAAGCT |
| | GCCTGAACTCCCGGGAGACTTCTTTCATCACCAATGGTTCCTGCCTTGCGTCCCGCAAG |
| | ACCTCTTTTATGATGGCCCTGTGCCTGTCAAGCATATACGAAGATCTGAAGATGTACCA |
| | AGTCGAGTTCAAGACCATGAATGCGAAGCTCCTTATGGACCCTAAGCGGCAGATCTTCC |
| | TGGATCAGAACATGCTGGCTGTGATCGACGAGCTGATGCAGGCTCTGAACTTCAACAG |
| | CGAGACAGTGCCGCAAAAGTCCAGCCTGGAAGAACCCGACTTCGCCAAGACCAAGATC |
| | AAGCTGTGCATTCTGCTGCACGCATTCAGGATCAGGGCAGTCACTATTGACAGAGTGAT |
| | GTCCTACCTGAACGCCAGC (SEQ ID NO: 49) |
| Nucleotide | GATATCCAGATGACCCAGAGCCCTAGCTCTCTGAGCGCCAGCGTGGGCGACAGAGTGA |
| sequence | CCATCACCTGTAGAGCCTCTCAGGGCATCTCCAACTACCTGGCTTGGTTCCAGCAGAAA |
| encoding | CCTGGCAAGGCCCCTAAGCGGCTGATCTACGCCGCTCAGATCCCAGGCAGCGGCGTC |
| TPP-77658 | CCCAGCAGATTCAGCGGCTCCGGCTCTGGCACCGACTTCACCCTGACCATTAGCAGCC |
| light chain; | TGCAACCTGAGGACTTTGCCACATATTACTGCCTGCAGCACTACAGCTACCCCCTGACA |
| also light | TTCGGCGGAGGAACAAAGGTGGAAATCAAGCGTACGGTGGCTGCACCATCTGTCTTCA |
| chain of | TCTTCCCGCCATCTGATGAGCAGTTGAAATCTGGAACTGCCTCTGTTGTGTGCCTGCTG |
| H10658 | AATAACTTCTATCCCAGAGAGGCCAAAGTACAGTGGAAGGTGGATAACGCCCTCCAATC |
| fusion (SEQ | GGGTAACTCCCAGGAGAGTGTCACAGAGCAGGACAGCAAGGACAGCACCTACAGCCT |
| ID NO: 6) | CAGCAGCACCCTGACGCTGAGCAAAGCAGACTACGAGAAACACAAAGTCTACGCCTGC |
| | GAAGTCACCCATCAGGGCCTGAGCTCGCCCGTCACAAAGAGCTTCAACAGGGGAGAGT |
| | GT (SEQ ID NO: 50) |

In some embodiments, the disclosure provides a polynucleotide or polynucleotides comprising one or more nucleotide sequences encoding any IL-12 variant provided herein. In some embodiments, the disclosure provides a polynucleotide or polynucleotides comprising one or more nucleotide sequences encoding the IL-12 variant H10, wherein the variant H10 comprises the p35 subunit amino acid sequence of SEQ ID NO: 3 and the p40 subunit amino acid sequence of SEQ ID NO: 4. In some embodiments, the disclosure provides a polynucleotide or polynucleotides comprising one or more nucleotide sequences encoding the IL-12 variant H1, H2, H3, H4, H5, H6, H7, H8, H9, H10, H11, H12, H13, H14, H15, H16, H17, H18, H19. H20, H21, H22, H23, H24, H25, H30, H31, or H32 as described in Example 1 herein.

In some embodiments, the disclosure provides a polynucleotide or polynucleotides comprising one or more nucleotide sequences encoding any anti-PD1 antibody provided herein. In some embodiments, the disclosure provides a polynucleotide or polynucleotides comprising one or more nucleotide sequences encoding an isolated antibody that binds to PD1 and comprises a VH and VL, wherein the VH comprises the amino acid sequence of SEQ ID NO: 7 and the VL comprises the amino acid sequence of SEQ ID NO: 8. In some embodiments, the disclosure provides a polynucleotide or polynucleotides comprising one or more nucleotide sequences encoding an isolated antibody that binds to PD1 and comprises a VH and VL, wherein the VH comprises the amino acid sequence of SEQ ID NO: 17 and the VL comprises the amino acid sequence of SEQ ID NO: 18. In some embodiments, the disclosure provides a polynucleotide or polynucleotides comprising one or more nucleotide sequences encoding an isolated antibody that binds to PD1 and comprises a VH and VL, wherein the VH comprises the amino acid sequence of SEQ ID NO: 33 and the VL comprises the amino acid sequence of SEQ ID NO: 34. In some embodiments, the disclosure provides a polynucleotide or polynucleotides comprising one or more nucleotide sequences encoding an isolated antibody that binds to PD1 and that comprises a light chain comprising the amino acid sequence of SEQ ID NO: 6 and a heavy chain comprising the amino acid sequence of SEQ ID NO: 5, 51, or 52, wherein the C-terminal lysine of SEQ ID NO: 5, 51, or 52 is optional. In some embodiments, the disclosure provides a polynucleotide or polynucleotides comprising one or more nucleotide sequences encoding an isolated antibody that binds to PD1 and that comprises a light chain comprising the amino acid sequence of SEQ ID NO: 16 and a heavy chain comprising the amino acid sequence of SEQ ID NO: 15, 53, or 54, wherein the C-terminal lysine of SEQ ID NO: 15, 53, or 54 is optional. In some embodiments, the disclosure provides a polynucleotide or polynucleotides comprising one or more nucleotide sequences encoding an isolated antibody that binds to PD1 and that comprises a light chain comprising the amino acid sequence of SEQ ID NO: 43 and a heavy chain comprising the amino acid sequence of SEQ ID NO: 42, 55, or 56, wherein the C-terminal lysine of SEQ ID NO: 42, 55, or 56 is optional.

In some embodiments, the disclosure provides a polynucleotide or polynucleotides comprising one or more nucleotide sequences encoding any IL-12 variant/anti-PD1 fusion protein provided herein. In some embodiments, the disclosure provides a polynucleotide or polynucleotides comprising one or more nucleotide sequences encoding an IL-12 variant/anti-PD1 fusion protein that comprises 1) an antibody heavy chain, 2) an antibody heavy chain linked to the p35 subunit of the H10 IL-12 mutein: 3) an antibody light chain; and 4) the p40 subunit of the H10 IL-12 mutein, wherein the antibody heavy chain comprises the amino acid sequence of SEQ ID NO: 5, wherein the antibody heavy chain linked to the p35 subunit of the H10 IL-12 mutein comprises the amino acid sequence of SEQ ID NO: 25, wherein the antibody light chain comprises the amino acid sequence of SEQ ID NO: 6, and wherein the p40 subunit of the H10 IL-12 mutein comprises the amino acid sequence of SEQ ID NO: 4. In some embodiments, the disclosure provides a polynucleotide or polynucleotides comprising one or more nucleotide sequences encoding an IL-12 variant/anti-PD1 fusion protein that comprises 1) an antibody heavy chain, 2) an antibody heavy chain linked to the p35 subunit of the H10 IL-12 mutein; 3) an antibody light chain; and 4) the p40 subunit of the H10 IL-12 mutein, wherein the antibody heavy chain comprises the amino acid sequence of SEQ ID NO: 15, wherein the antibody heavy chain linked to the p35 subunit of the H10 IL-12 mutein comprises the amino acid sequence of SEQ ID NO: 26, wherein the antibody light chain comprises the amino acid sequence of SEQ ID NO: 16, and wherein the p40 subunit of the H10 IL-12 mutein comprises the amino acid sequence of SEQ ID NO: 4.

In some embodiments, provided herein is a polynucleotide comprising the nucleic acid sequence of the insert of the plasmid deposited with the ATCC and having Accession No. PTA-127517 encoding the TPP-77658 heavy chain of the H10658 fusion. In some embodiments, provided herein is a polynucleotide comprising the nucleic acid sequence of the insert of the plasmid deposited with the ATCC and having Accession No. PTA-127518 encoding the TPP-77658 heavy chain-H10 mutein p35 fusion polypeptide of the H10658 fusion. In some embodiments, provided herein is a polynucleotide comprising the nucleic acid sequence of the insert of the plasmid deposited with the ATCC and having Accession No. PTA-127519 encoding the TPP-77658 light chain of the H10658 fusion. In some embodiments, provided herein is a polynucleotide comprising the nucleic acid sequence of the insert of the plasmid deposited with the ATCC and having Accession No. PTA-127520 encoding the H10 mutein p40 subunit of the H10658 fusion.

In addition, also provided herein is a polypeptide comprising the amino acid sequence encoded by the DNA insert of the plasmid deposited with the ATCC and having Accession No. PTA-127517, encoding the TPP-77658 heavy chain of the H10658 fusion. Also provided herein is a polypeptide comprising the amino acid sequence encoded by the DNA insert of the plasmid deposited with the ATCC and having Accession No. PTA-127518, encoding the TPP-77658 heavy chain-H10 mutein p35 fusion of the H10658 fusion. Also provided herein is a polypeptide comprising the amino acid sequence encoded by the DNA insert of the plasmid deposited with the ATCC and having Accession No. PTA-127519, encoding the TPP-77658 light chain of the H10658 fusion. Also provided herein is a polypeptide comprising the amino acid sequence encoded by the DNA insert of the plasmid deposited with the ATCC and having Accession No. PTA-127520, encoding the H10 mutein p40 subunit of the H10658 fusion.

In some embodiments, provided herein is an anti-PD1 antibody comprising a VH encoded by a portion of the DNA insert of the plasmid deposited with the ATCC and having Accession No. PTA-127517 and a VL encoded by a portion of the DNA insert of the plasmid deposited with the ATCC and having Accession No. PTA-127519. In some embodiments, provided herein is an anti-PD1 antibody comprising a heavy chain encoded by the DNA insert of the plasmid deposited with the ATCC and having Accession No. PTA-127517 and a light chain encoded by the DNA insert of the plasmid deposited with the ATCC and having Accession No. PTA-127519. In some embodiments, provided herein is an anti-PD1 antibody comprising a heavy chain encoded by the DNA insert of the plasmid deposited with the ATCC and having Accession No. PTA-127518 and a light chain encoded by the DNA insert of the plasmid deposited with the ATCC and having Accession No. PTA-127519. In some embodiments, provided herein is an IL-12 variant comprising a p35 subunit encoded by a portion of the DNA insert of the plasmid deposited with the ATCC and having Accession No. PTA-127518 and a p40 subunit encoded by the DNA insert of the plasmid deposited with the ATCC and having Accession No. PTA-127520.

It will be appreciated by those of ordinary skill in the art that, as a result of the degeneracy of the genetic code, there are many nucleotide sequences that encode a polypeptide as described herein. Some of these polynucleotides bear minimal homology to a nucleotide sequence provided herein. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide chain.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae. S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Pharmaceutical Compositions

In another embodiment, the invention comprises pharmaceutical compositions.

A “pharmaceutical composition” refers to a mixture of an IL-12 variant, anti-PD1 antibody, or fusion protein of the invention and one or more excipient.

Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol: alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Therapeutic, Diagnostic, and Other Methods

IL-12 variants, anti-PD1 antibodies, and IL-12 variant/anti-PD1 fusion proteins of the present invention are useful in various applications including but not limited to, as a medicament, for therapeutic treatment methods and for diagnostic methods.

In some embodiments, IL-12 variants and IL-12 variant/anti-PD1 fusion proteins provided herein may be used for treating a subject for any condition in which it would be beneficial to have increased IL-12 activity. IL-12 variants and IL-12 variant/anti-PD1 fusion proteins provided herein are particularly useful for situations in which it is desirable to provide IL-12 activity to a subject in a tightly controlled or limited approach.

In one aspect, the invention provides a method for treating cancer. In some embodiments, the method of treating cancer in a subject comprises administering to the subject in need thereof an effective amount of a pharmaceutical composition comprising any of the IL-12 variant, antibodies, or fusion proteins as described herein. In some embodiments, provided is a method of treating cancer in a subject, comprising administering to the subject in need thereof an effective amount of a composition comprising an IL-12 variant, antibodies, or fusion protein provided herein.

In some embodiments, provided herein is a method of inhibiting the growth of PD1/PD-L1-treatment resistant cancer cells in a subject, comprising administering to the subject in need thereof an effective amount of a composition comprising an IL-12 variant or IL-12 variant/anti-PD1 fusion protein provided herein.

In some embodiments, provided herein is a method of promoting the infiltration of CD8 positive (+) T cells in the tumor microenvironment in a subject, comprising administering to the subject in need thereof an effective amount of a composition comprising an IL-12 variant or IL-12 variant/anti-PD1 fusion protein provided herein.

In some embodiments, provided herein is a method of promoting STAT4 phosphorylation in a subject, comprising administering to the subject in need thereof an effective amount of a composition comprising an IL-12 variant or IL-12 variant/anti-PD1 fusion protein provided herein.

In some embodiments, provided herein is a method of promoting the production of interferon gamma (IFNg) in a subject, comprising administering to the subject in need thereof an effective amount of a composition comprising an IL-12 variant or IL-12 variant/anti-PD1 fusion protein provided herein.

In another aspect, the invention further provides an IL-12 variant, IL-12 variant/anti-PD1 fusion protein or related pharmaceutical composition as described herein for use in the described method of treating cancer. The invention also provides the use of an IL-12 variant or IL-12 variant/anti-PD1 fusion protein as described herein in the manufacture of a medicament for treating cancer.

In some embodiments, cancers that can be treated with IL-12 variants, anti-PD1 antibodies, or IL-12 variant/anti-PD1 fusion proteins provided herein include, for example, solid tumors or liquid tumors. In some embodiments, solid tumors for treatment with an IL-12 variant provided herein include, for example, non-small-cell lung cancer (NSCLC), ovarian cancer, renal cell carcinoma (RCC), colorectal cancer (CRC), and hepatocellular carcinoma (HCC). In some embodiments, cancers that can be treated include one or more of bladder cancer, breast cancer, clear cell kidney cancer, head/neck squamous cell carcinoma (HNSCC) [squamous cell carcinoma of the head and neck (SCCHN)], lung squamous cell carcinoma, lung adenocarcinoma, malignant melanoma, non-small-cell lung cancer (NSCLC), ovarian cancer, pancreatic cancer, prostate cancer, renal cell carcinoma (RCC), small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Md-1), myelodysplastic syndrome (MDS), non-Hodgkin's lymphoma (NHL), small lymphocytic lymphoma (SLL), endometrial cancer, B-cell acute lymphoblastic leukemia, colorectal cancer (CRC), glioblastoma, uterine cancer, cervical cancer, penile cancer, gastric cancer (GC), and non-melanoma skin cancer. In some embodiments, cancers that can be treated with IL-12 variants, anti-PD1 antibodies, or IL-12 variant/anti-PD1 fusion proteins provided herein include, for example, NSCLC that has been previously treated with platinum-based treatment and/or a checkpoint inhibitor (e.g. a PD(L)1 inhibitor), RCC that has been previously treated with a tyrosine kinase inhibitor and/or a checkpoint inhibitor (e.g. a PD(L)1 inhibitor), ovarian cancer, microsatellite stable (MSS) CRC, hepatocellular carcinoma (HCC), or bladder cancer.

### Administration and Dosing

Typically, an IL-12 variant, anti-PD1 antibody, or IL-12 variant/anti-PD1 fusion protein of the invention is administered in an amount effective to treat a condition as described herein. The molecules the invention can be administered as a molecule per se, or alternatively, as a pharmaceutical composition containing the molecule.

The molecules of the invention are administered by any suitable route in the form of a pharmaceutical composition adapted to such a route, and in a dose effective for the treatment intended.

In some embodiments, the antibodies may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques. In some embodiments, an IL-12 variant, anti-PD1 antibody, or IL-12 variant/anti-PD1 fusion protein provided herein is administered subcutaneously (SC). In some embodiments, an IL-12 variant, anti-PD1 antibody, or IL-12 variant/anti-PD1 fusion protein provided herein is administered intravenously (IV).

The dosage regimen for the antibodies of the invention or compositions containing said antibodies is based on a variety of factors, including the type, age, weight, sex and medical condition of the subject; the severity of the condition; the route of administration; and the activity of the particular antibody employed. Thus, the dosage regimen may vary widely. In one embodiment, the total daily dose of an antibody of the invention is typically from about 0.01 to about 100 mg/kg (i.e., mg antibody of the invention per kg body weight) for the treatment of the indicated conditions discussed herein. In another embodiment, total daily dose of the antibody of the invention is from about 0.1 to about 20 mg/kg, and in another embodiment, from about 0.5 to about 10 mg/kg.

In some embodiments, an IL-12 variant, anti-PD1 antibody, or IL-12 variant/anti-PD1 fusion protein provided herein is administered once a week (Q1W), once every two weeks (Q2W), once every three weeks (Q3W), once every four weeks (Q4W), once every five weeks (Q5W), once every six weeks (Q6W), once a month (Q1M), once every two months (Q2M), or once every three months (Q3M).

In some embodiments, an IL-12 variant/anti-PD1 fusion protein is provided at a dose of about 1 mg/kg, 2 mg/kg, 3 mg/kg, 5 mg/kg, 10 mg/kg, 15 mg/kg, or 20 mg/kg.

In some embodiments, an IL-12 variant/anti-PD1 fusion protein is provided IV Q2W at a dose of 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg or 15 mg/kg. In some embodiments, an IL-12 variant/anti-PD1 fusion protein is provided IV Q3W at a dose of 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg or 15 mg/kg. In some embodiments, an IL-12 variant/anti-PD1 fusion protein is provided SC Q2W at a dose of 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg or 15 mg/kg. In some embodiments, an IL-12 variant/anti-PD1 fusion protein is provided SC Q3W at a dose of 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg or 15 mg/kg. In some embodiments, the IL-12 variant/anti-PD1 fusion protein provided at the doses above is the H10868 fusion or the H10658 fusion described in Example 3 herein.

Toxicity and efficacy of the prophylactic and/or therapeutic protocols of the instant invention maybe determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it may be expressed as the ratio LD₅₀/ED₅₀. Prophylactic and/or therapeutic agents that exhibit large therapeutic indices are preferred.

### Co-Administration

The IL-12 variants, anti-PD1 antibodies, and IL-12 variant/anti-PD1 fusion proteins provided herein can be used alone, or in combination with one or more other therapeutic agents. Provided herein are any of the uses, methods or compositions as defined herein wherein an IL-12 variants, anti-PD1 antibodies, or IL-12 variant/anti-PD1 fusion protein of the invention is used in combination with one or more other therapeutic agent discussed herein.

The administration of two or more agents “in combination” means that all of the agents are administered closely enough in time to affect treatment of the subject. The two or more agents may be administered simultaneously or sequentially. Additionally, simultaneous administration may be carried out by mixing the agents prior to administration or by administering the agents at the same point in time but as separate dosage forms at the same or different site of administration.

In some embodiments, an IL-12 variant, anti-PD1 antibody, or IL-12 variant/anti-PD1 fusion protein provided herein may be administered in combination with the administration of one or more additional therapeutic agents. Optionally, the additional therapeutic agent may include an additional anti-cancer agent. These include, but are not limited to, the administration of a biotherapeutic agent and/or a chemotherapeutic agent, such as but not limited to, a vaccine, a CAR-T cell-based therapy, radiotherapy, a cytokine therapy, a CD3 bispecific antibody, an inhibitor of other immunosuppressive pathways, an inhibitor of angiogenesis, a T cell activator, an inhibitor of a metabolic pathway, an mTOR inhibitor, an inhibitor of an adenosine pathway, a tyrosine kinase inhibitor including but not limited to Inlyta, ALK inhibitors and sunitinib, a BRAF inhibitor, an epigenetic modifier, an IDO1 inhibitor, a JAK inhibitor, a STAT inhibitor, a cyclin-dependent kinase inhibitor, a biotherapeutic agent (including but not limited to antibodies to VEGF, VEGFR, EGFR, Her2/neu, other growth factor receptors, CD40, CD-40L, CTLA-4, OX-40, 4-1BB, TIGIT, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of biotherapeutic agents include therapeutic antibodies, immune modulating agents, and therapeutic immune cells.

Therapeutic antibodies may have specificity against a variety of different of antigens. For example, therapeutic antibodies may be directed to a tumor associated-antigen, such that binding of the antibody to the antigen promotes death of the cell expressing the antigen. In other example, therapeutic antibodies may be directed to an antigen (e.g. PD1) on an immune cell, such that binding of the antibody prevents downregulation of the activity of the cell expressing the antigen (and thereby promotes activity of the cell expressing the antigen). In another example, a therapeutic activity may be directed to an antigen such that binding of the antibody to the antigen stimulates the target molecule that contains the antigen (i.e. the antibody is an agonist antibody), in order to promote a desired activity. In some situations, a therapeutic antibody may function through multiple different mechanisms (for example, it may both i) promote death of the cell expressing the antigen, and ii) prevent the antigen from causing down-regulation of the activity of immune cells in contact with the cell expressing the antigen).

Therapeutic antibodies may be directed to, for example, the antigens listed as follows. For some antigens, exemplary antibodies directed to the antigen are also included below (in brackets/parenthesis after the antigen). The antigens as follow may also be referred to as “target antigens” or the like herein. Target antigens for therapeutic antibodies herein include, for example: 4-1BB (e.g. utomilumab); 5T4; A33; alpha-folate receptor 1 (e.g. mirvetuximab soravtansine); Alk-1; BCMA [e.g. PF-06863135 (see U.S. Pat. No. 9,969,809)]; BTN1A1 (e.g. see WO2018222689); CA-125 (e.g. abagovomab); Carboanhydrase IX; CCR2; CCR4 (e.g. mogamulizumab); CCR5 (e.g. leronlimab); CCR8; CD3 [e.g. blinatumomab (CD3/CD19 bispecific), PF-06671008 (CD3/P-cadherin bispecific), PF-06863135 (CD3/BCMA bispecific), CD19 (e.g. blinatumomab, MOR208); CD20 (e.g. ibntumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, isatuximab), CD40; CD-40L; CD44v6; CD47; CD52 (e.g. alemtuzumab); CD63; CD79 (e.g. polatuzumab vedotin); CD80; CD123; CD276/B7-H3 (e.g. omburtamab); CDH17; CEA; ClhCG, CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO20181220584); GD2 (e.g. dinutuximab, 3F8); GD3; GITR; GloboH; GM1; GM2; GUCY2C (e.g. PF-07062119); HER2/neu [e.g. margetuximab, pertuzumab, trastuzumab; ado-trastuzumab emtansine, trastuzumab duocarmazine, PF-06804103 (see U.S. Pat. No. 8,828,401)]; HER3; HER4; ICOS; IL-10; ITG-AvB6; LAG-3 (e.g. relatlimab); Lewis-Y; LG; Ly-6; M-CSF [e.g. PD-0360324 (see U.S. Pat. No. 7,326,414)]; MCSP; mesothelin; MUC1; MUC2; MUC3; MUC4; MUC5AC; MUC5B; MUC7; MUC16; Notch1; Notch3; Nectin-4 (e.g. enfortumab vedotin); OX40 [e.g. PF-04518600 (see U.S. Pat. No. 7,960,515)]; P-Cadherin [e.g. PF-06671008 (see WO2016/001810)]; PCDHB2; PD1 [e.g. BCD-100, camrelizumab, cemiplimab, genolimzumab (CBT-501), MED10680, nivolumab, pembrolizumab, RN888 (see WO2016/092419), sintilimab, spartalizumab. STI-A1110, tislelizumab, TSR-042]; PD-L1 (e.g. atezolizumab, durvalumab, BMS-936559 (MDX-1105), or LY3300054); PDGFRA (e.g. olaratumab); Plasma Cell Antigen; PolySA; PSCA; PSMA; PTK7 [e.g. PF-06647020 (see U.S. Pat. No. 9,409,995)]; Ror1; SAS; SCRx6; SLAMF7 (e.g. elotuzumab); SHH; SIRPa (e.g. ED9, Effi-DEM); STEAP; TGF-beta; TIGIT; TIM-3; TMPRSS3; TNF-alpha precursor; TROP-2 (e.g sacituzumab govitecan); TSPAN8; VEGF (e.g. bevacizumab, brolucizumab): VEGFR1 (e.g. ranibizumab); VEGFR2 (e.g. ramucirumab, ranibizumab); Wue-1.

Therapeutic antibodies administered in combination with the IL-12 variants, anti-PD1 antibodies, or IL-12 variant/anti-PD1 fusion proteins provided herein may have any suitable format. For example, therapeutic antibodies may have any format as described elsewhere herein. In some embodiments, a therapeutic antibody may be a naked antibody. In some embodiments, a therapeutic antibody may be linked to a drug or other agent (also known as an “antibody-drug conjugate” (ADC)). In some embodiments, a therapeutic antibody against a particular antigen may incorporated into a multi-specific antibody (e.g. a bispecific antibody).

In some embodiments, an IL-12 variant, anti-PD1 antibody, or IL-12 variant/anti-PD1 fusion protein provided herein provided herein may be administered in combination with a pattern recognition receptor (PRR) agonists, immunostimulatory cytokines, and cancer vaccines. There are multiple classes of PRR molecules, including toll-like receptors (TLRs), RIG-1-like receptors (RLRs), nucleotide-binding oligomerization domain (NOD)-like receptors (NLRs), C-type lectin receptors (CLRs), and Stimulator of Interferon Genes (STING) protein. Other PRRs include, for example, DNA-dependent Activator of IFN-regulatory factors (DAI) and Absent in Melanoma 2 (AIM2). In some embodiments, an IL-12 variant, anti-PD1 antibody, or IL-12 variant/anti-PD1 fusion protein provided herein may be administered in combination with a TLR agonist (e.g. TLR2, TLR3, TLR4, TLR5, TLR6, TLR7, TLR8, or TLR9 agonist).

Examples of immunostimulatory cytokines that are useful in the treatment methods, medicaments, and uses of the present invention include GM-CSF, G-CSF, IFN-alpha, IFN-gamma; IL-2 (e.g. denileukin diftitox), IL-6, IL-7, IL-11, IL-15, IL-18, IL-21, and TNF-alpha.

Examples of cancer vaccines that are useful in the treatment methods, medicaments, and uses of the present invention include, for example, sipuleucel-T and talimogene laherparepvec (T-VEC).

Examples of immune cell therapies that are useful in the treatment methods, medicaments, and uses of the present invention include, for example, tumor-infiltrating lymphocytes (TILs) and chimeric antigen receptor T cells (CAR-T cells).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, nitrosureas such as carmustine, chlorozotocin, fotemustine, lomustine, nimustine, ranimustine; antibiotics such as the enediyne antibiotics (e.g., calicheamicin, especially calicheamicin gamma1I and calicheamicin phil1, see, e.g., Agnew, Chem. Inti. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), pegylated liposomal doxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone, mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16): ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; KRAS inhibitors; MCT4 inhibitors; MAT2a inhibitors; tyrosine kinase inhibitors such as sunitinib, axitinib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600; erb inhibitors such as dacomitinib; PARP inhibitors such as talazoparib; SMO inhibitors such as glasdegib, PF-5274857; EGFR T790M inhibitors such as PF-06747775; EZH2 inhibitors such as PF-06821497; PRMT5 inhibitors such as PF-06939999; TGFRβr1 inhibitors such as PF-06952229; and pharmaceutically acceptable salts, acids or derivatives of any of the above. In specific embodiments, such additional therapeutic agent is bevacizumab, cetuximab, sirolimus, panitumumab, 5-fluorouracil (5-FU), capecitabine, tivozanib, irinotecan, oxaliplatin, cisplatin, trifluridine, tipiracil, leucovorin, gemcitabine, regorafenib or erlotinib hydrochloride.

In some embodiments, an IL-12 variant, anti-PD1 antibody, or IL-12 variant/anti-PD1 fusion protein provided herein is administered in combination with a PD1 or PDL1 inhibitor. PD1 and PDL1 inhibitors are collectively referred to herein as “PD(L)1” inhibitors. In some embodiments, the PD(L)1 inhibitor is sasanlimab.

In some embodiments, PD(L)1 inhibitors are anti-PD1 or anti-PD-L1 antibodies. These bind to PD1 or PDL1, and block the interaction between PD1 and PDL1. Examples of PD(L)1 inhibitors useful in methods, medicaments and uses of the present invention include, for example: sasanlimab (aka RN888, an anti-PD1 IgG4 monoclonal antibody) pembrolizumab (aka MK-3475, an anti-PD1 IgG4 monoclonal antibody) nivolumab (aka BMS-936558 or MDX1106, an anti-PD1 IgG4 monoclonal antibody), cemiplimab (aka REGN-2810, an anti-PD1 antibody), atezolizumab (aka MPDL3280A an IgG1-engineered, anti-PDL1 antibody), BMS-936559 (a fully human, anti-PDL1, IgG4 monoclonal antibody), MED14736 (aka durvalumab, an engineered IgG1 kappa anti-PDL1 monoclonal antibody with triple mutations in the Fc domain to remove antibody-dependent, cell-mediated cytotoxic activity). Additional exemplary PD(L)1 inhibitors useful in the methods, medicaments and uses of the present invention include SHR1210 (anti-PD1 antibody), KN035 (anti-PDL1 antibody), IB1308 (anti-PD1 antibody), PDR001 (anti-PD1 antibody), BGB-A317 (anti-PD1 antibody), BCD-100 (anti-PD1 antibody), JS001 (anti-PD1 antibody). In some embodiments, a PD(L)1 inhibitor is a small molecule PD1 or PDL1 antagonist (e.g. CA-170), as described in Yang et al Med. Res. Rev. (2019), 39, pp 265-301.

In some circumstances, it may be advantageous to combine a IL-12 variant/anti-PD1 fusion protein provided herein with an anti-PD1 antibody that binds to a different epitope on PD1 than the anti-PD1 antibody of the IL-12 variant/anti-PD1 fusion protein. For example, some anti-PD1 antibodies provided herein bind to an epitope on P01 such that the antibody does not block the interaction between PD1 and PDL1. In contrast, most or all anti-PD1 antibodies previously approved for therapeutic use bind to an epitope on PD1 such that the antibody inhibits the interaction between PD1 and PDL1.

Anti-PD1 antibodies that do not block the interaction between PD1 and PDL1 are useful for targeting an IL-12 variant/anti-PD1 fusion protein to PD1 expressing cells, such as T cells in the tumor microenvironment.

Thus, in some embodiments, provided herein is a combination therapy comprising 1) an IL-12 variant/anti-PD1 fusion protein provided herein wherein the anti-PD1 antibody of the fusion protein does not block the interaction between PD1 and PDL1 and 2) a PD(L)1 inhibitor, wherein the PD(L)1 inhibitor blocks the interaction between PD1 and PDL1. In some embodiments, the PD(L)1 inhibitor is an anti-PD1 antibody that inhibits the interaction between PD1 and PDL1. In some embodiments, the PD(L)1 inhibitor is an anti-PDL1 antibody that inhibits the interaction between PD1 and PDL1.

In some embodiments, an IL-12 variant, anti-PD1 antibody, or IL-12 variant/anti-PD1 fusion protein provided herein administered in combination with an VEGF or VEGF receptor (VEGFR) inhibitor. VEGF and VEGFR inhibitors are collectively referred to herein as “VEGF(R)” inhibitors. VEGF(R) inhibitors include agents that bind to any VEGF subtype (e.g. VEGF-A, VEGF-C, and VEGF-D) and VEGFR subtype (e.g. VEGFR1, VEGFR2, and VEGFR3). In some embodiments, the VEGF(R) inhibitor is axitinib or bevacizumab.

In some embodiments, VEGF(R) inhibitors are anti-VEGF or anti-VEGFR antibodies. These bind to VEGF or VEGFR, and block the interaction between VEGF and VEGFR and/or inhibit the activity of VEGFR. Anti-VEGF(R) antibodies include, for example, bevacizumab, ramucirumab, and ranibizumab. In some embodiments. VEGF(R) inhibitors are small molecule agents that bind to VEGF or VEGFR and inhibit the activity of VEGFR. Small molecule VEGF(R) inhibitors include, for example, apatinib, axitinib, cabozantinib, lapatinib, lenvatinib, nintedanib, pazopanib, ponatinib, regorafenib, sorafenib, sunitinib, and vandetanib.

In some embodiments, an IL-12 variant, anti-PD1 antibody, or IL-12 variant/anti-PD1 fusion protein provided herein may be co-administered with, or be sequentially administered before or after the other agent treatment by intervals ranging from minutes to weeks. In embodiments where the other agents and/or a proteins or polynucleotides are administered separately, one would generally ensure that a significant period of time did not expire between each delivery, such that the agent and the composition of the present invention would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

In some embodiments, an IL-12 variant, anti-PD1 antibody, or IL-12 variant/anti-PD1 fusion protein is combined with a treatment regimen further comprising a traditional therapy selected from the group consisting of surgery, radiation therapy, chemotherapy, targeted therapy, immunotherapy, hormonal therapy, angiogenesis inhibition and palliative care.

### Kits

Another aspect of the invention provides kits comprising an IL-12 variant, anti-PD1 antibody, or IL-12 variant/anti-PD1 fusion protein provided herein. A kit may include, in addition to an IL-12 variant, anti-PD1 antibody, or IL-12 variant/anti-PD1 fusion protein, one or more diagnostic or therapeutic agents. A kit may also include instructions for use in a diagnostic or therapeutic method. In some embodiments, the kit includes an antibody or a pharmaceutical composition thereof and a diagnostic agent. In other embodiments, the kit includes an antibody or a pharmaceutical composition thereof and one or more therapeutic agents, such as a PD(L)1 inhibitor (e.g. a blocking anti-PD1 antibody).

In yet another embodiment, the invention comprises kits that are suitable for use in performing the methods of treatment described herein. In one embodiment, the kit contains a first dosage form comprising one or more of the IL-12 variants, anti-PD1 antibodies, or IL-12 variant/anti-PD1 fusion proteins of the invention in quantities sufficient to carry out the methods of the invention. In another embodiment, the kit comprises one or more of the IL-12 variants, anti-PD1 antibodies, or IL-12 variant/anti-PD1 fusion proteins of the invention in quantities sufficient to carry out the methods of the invention and at least a first container for a first dosage and a second container for a second dosage.

### Biological Deposits

Representative materials of the present invention were deposited in the American Type Culture Collection (ATCC), 10801 University Boulevard, Manassas, VA 20110-2209, USA, on Feb. 7, 2023. Vector “HC_H10658” having ATCC Accession No. PTA-127517 comprises a DNA insert encoding the TPP-77658 heavy chain of the H10658 fusion. Vector “HCp35_H10658” having ATCC Accession No. PTA-127518 comprises a DNA insert encoding the TPP-77658 heavy chain-H10 mutein p35 fusion of the H10658 fusion. Vector “LC_H10658” having ATCC Accession No. PTA-127519 comprises a DNA insert encoding the TPP-77658 light chain of the H10658 fusion. Vector “p40_H10658” having ATCC Accession No. PTA-127520 comprises a DNA insert encoding the H10 mutein p40 subunit of the H10658 fusion. These are summarized in Table 8 below.

TABLE 8
ATCC deposit information

| | | ATCC |
|---|---|---|
| Vector | Description | Accession No. |
| HC_H10658 | TPP-77658 heavy chain | PTA-127517 |
| | of the H10658 fusion | |
| HCp35_H10658 | TPP-77658 heavy chain-H10 mutein p35 | PTA-127518 |
| | fusion polypeptide of the H10658 fusion | |
| LC_H10658 | TPP-77658 light chain | PTA-127519 |
| | of the H10658 fusion | |
| p40_H10658 | H10 mutein p40 subunit | PTA-127520 |
| | of the H10658 fusion | |

The deposits were made under the provisions of the Budapest Treaty on the International Recognition of the Deposit of Microorganisms for the Purpose of Patent Procedure and Regulations thereunder (Budapest Treaty). This assures maintenance of a viable culture of the deposit for 30 years from the date of deposit. The deposit will be made available by ATCC under the terms of the Budapest Treaty, and subject to an agreement between Pfizer Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 U.S.C. Section 122 and the Commissioner's rules pursuant thereto (including 37 C.F.R. Section 1.14 with particular reference to 886 OG 638).

The assignee of the present application has agreed that if a culture of the materials on deposit should die or be lost or destroyed when cultivated under suitable conditions; the materials will be promptly replaced on notification with another of the same. Availability of the deposited material is not to be construed as a license to practice the invention in contravention of the rights granted under the authority of any government in accordance with its patent laws

Incorporated by reference herein for all purposes is the content of U.S. Provisional Patent Application No. 63/353,241 filed Jun. 17, 2022 and U.S. Provisional Patent Application No. 63/496,545 filed Apr. 17, 2023.

### Summary of Sequences

Sequences provided in this application are summarized in Table 9 below.

TABLE 9

| SEQ | | |
|---|---|---|
| ID | | |
| NO: | Description | Sequence |
| 1 | WT human | RNLPVATPDPGMFPCLHHSQNLLRAVSNMLQKARQTLEFYPCTSEEIDHEDITK |
| | mature IL12 | DKTSTVEACLPLELTKNESCLNSRETSFITNGSCLASRKTSFMMALCLSSIYEDL |
| | p35 | KMYQVEFKTMNAKLLMDPKRQIFLDQNMLAVIDELMQALNFNSETVPQKSSLEE |
| | | PDFYKTKIKLCILLHAFRIRAVTIDRVMSYLNAS |
| 2 | WT human | IWELKKDVYVVELDWYPDAPGEMVVLTCDTPEEDGITWTLDQSSEVLGSGKTL |
| | mature IL 12 | TIQVKEFGDAGQYTCHKGGEVLSHSLLLLHKKEDGIWSTDILKDQKEPKNKTFL |
| | p40 | RCEAKNYSGRFTCWWLTTISTDLTFSVKSSRGSSDPQGVTCGAATLSAERVRG |
| | | DNKEYEYSVECQEDSACPAAEESLPIEVMVDAVHKLKYENYTSSFFIRDIIKPDP |
| | | PKNLQLKPLKNSRQVEVSWEYPDTWSTPHSYFSLTFCVQVQGKSKREKKDRV |
| | | FTDKTSATVICRKNASISVRAQDRYYSSSWSEWASVPCS |
| 3 | p35 H10 | RNLPVATPDPGMFPCLHHSQNLLRAVSNMLQKARQTLEFYPCTSEEIDHEDITK |
| | mutein | DKTSTVEACLPLELTKNESCLNSRETSFITNGSCLASRKTSFMMALCLSSIYEDL |
| | | KMYQVEFKTMNAKLLMDPKRQIFLDQNMLAVIDELMQALNFNSETVPQKSSLEE |
| | | PDFAKTKIKLCILLHAFRIRAVTIDRVMSYLNAS |
| 4 | p40 H10 | IWELKKDVYVVELDWYPDAPGEMVVLTCDTPEEDGITWTLDQSSEVLGSGKTL |
| | mutein; | TIQVKEFGDAGQYTCHKGGEVLSHSLLLLHKKEDGIWSTLILKDQKEPKNKTFLR |
| | component of | CEAKNYSGRFTCWWLTTISTDLTFSVKSSRGSSDPQGVTCGAATLSAERVRGD |
| | H10658 | NKEYEYSVECQEDSACPAAEESLPIEVMVDAVHKLKYENYTSSFFIRDIIKPDPP |
| | fusion and | KNLQLKPLKNSRQVEVSWEYPDTWSTPHSYFSLTFCVQVQGGGGDRVFTDKT |
| | H10868 | SATVICRKNASISVRAQDRYYSSSWSEWASVPCS |
| | fusion | |
| 5 | TPP-77658 | EVQLVESGGGLVQPGGSLRLSCAASGFSFGDFDMRWFRQAPGKGLEWVGTIK |
| | Heavy Chain; | SRAYLEATEFAASVEGRFTISRDDAKNSAYLQMNSLRAEDTAVYYCTRDAYSS |
| | component of | GLLDYWGQGTLVTVSSASTKGPSVFPLAPSSKSTSGGTAALGCLVKDYFPEPV |
| | H10658 | TVSWNSGALTSGVHTFPAVLQSSGLYSLSSVVTVPSSSLGTQTYICNVNHKPSN |
| | fusion | TKVDKKVEPKSCDKTHTCPPCPAPEAAGAPSVFLFPPKPKDTLMISRTPEVTCV |
| | | WVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPCREEMTKNQVSLSCA |
| | | VKGFYPSDIAVEWESNGQPENNYKTTPPVLDSDGSFFLVSKLTVDKSRWQQG |
| | | NVFSCSVMHEALHNHYTQKSLSLSPGK |
| 6 | TPP-77658 | DIQMTQSPSSLSASVGDRVTITCRASQGISNYLAWFQQKPGKAPKRLIYAAQIP |
| | Light Chain; | GSGVPSRFSGSGSGTDFTLTISSLQPEDFATYYCLQHYSYPLTFGGGTKVEIKR |
| | component of | TVAAPSVFIFPPSDEQLKSGTASVVCLLNNFYPREAKVQWKVDNALQSGNSQE |
| | H10658 | SVTEQDSKDSTYSLSSTLTLSKADYEKHKVYACEVTHQGLSSPVTKSFNRGEC |
| | fusion | |
| 7 | TPP-77658 | EVQLVESGGGLVQPGGSLRLSCAASGFSFGDFDMRWFRQAPGKGLEVWGTIK |
| | VH | SRAYLEATEFAASVEGRFTISRDDAKNSAYLQMNSLRAEDTAVYYCTRDAYSS |
| | | GLLDYWGQGTLVTVSS |
| 8 | TPP-77658 | DIQMTQSPSSLSASVGDRVTITCRASQGISNYLAWFQQKPGKAPKRLIYAAQIP |
| | VL | GSGVPSRFSGSGSGTDFTLTISSLQPEDFATYYCLQHYSYPLTFGGGTKVEIK |
| 9 | TPP-77658 | GFSFGDFDMR |
| | HCDR1 | |
| | Extended | |
| 10 | TPP-77658 | TIKSRAYLEATEFAASVEG |
| | HCDR2, | |
| | Kabat and | |
| | Extended | |
| 11 | TPP-77658 | DAYSSGLLDY |
| | HCDR3 | |
| 12 | TPP-77658 | RASQGISNYLA |
| | LCDR1 | |
| 13 | TPP-77658 | AAQIPGS |
| | LCDR2 | |
| 14 | TPP-77658 | LQHYSYPLT |
| | LCDR3 | |
| 15 | TPP-76868 | EVQLVESGGGLVQPGGSLRLSCAASGFSFGDFDMRWFRQAPGKGLEWWVGLIK |
| | Heavy chain; | SRAYLEATEFAASVEGRFTISRDDAKNSAYLQMNSLRAEDTAVYYCTRDSYSS |
| | component of | GLLDYWGQGTLVTVSSASTKGPSVFPLAPSSKSTSGGTAALGCLVKDYFPEPV |
| | H10868 | TVSWNSGALTSGVHTFPAVLQSSGLYSLSSVVTVPSSSLGTQTYICNVNHKPSN |
| | fusion | TKVDKKVEPKSCDKTHTCPPCPAPEAAGAPSVFLFPPKPKDTLMISRTPEVTCV |
| | | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPCREEMTKNQVSLSCA |
| | | VKGFYPSDIAVEWESNGQPENNYKTTPPVLDSDGSFFLVSKLTVDKSRWQQG |
| | | NVFSCSVMHEALHNHYTQKSLSLSPGK |
| 16 | TPP-76868 | DIQMTQSPSSLSASVGDRVTITCRASQGISNYLAWFQQKPGKAPKRLIYAAQIP |
| | Light chain; | GSGVPSRFSGSGSGTDFTLTISSLQPEDFATYYCLQHYSYPLTFGGGTKVEIKR |
| | component of | TVAAPSVFIFPPSDEQLKSGTASVVCLLNNFYPREAKVQWKVDNALQSGNSQE |
| | H10868 | SVTEQDSKDSTYSLSSTLTLSKADYEKHKVYACEVTHQGLSSPVTKSFNRGEC |
| | fusion | |
| 17 | TPP-76868 | EVQLVESGGGLVQPGGSLRLSCAASGFSFGDFDMRWFRQAPGKGLEWWVGLIK |
| | VH | SRAYLEATEFAASVEGRFTISRDDAKNSAYLQMNSLRAEDTAVYYCTRDSYSS |
| | | GLLDYWGQGTLVTVSS |
| 18 | TPP-76868 | DIQMTQSPSSLSASVGDRVTITCRASQGISNYLAWFQQKPGKAPKRLIYAAQIP |
| | VL | GSGVPSRFSGSGSGTDFTLTISSLQPEDFATYYCLQHYSYPLTFGGGTKVEIK |
| 19 | TPP-76868 | GFSFGDFDMR |
| | HCDR1, | |
| | extended | |
| 20 | TPP-76868 | LIKSRAYLEATEFAASVEG |
| | HCDR2, | |
| | Kabat and | |
| | Extended | |
| 21 | TPP-76868 | DSYSSGLLDY |
| | HCDR3 | |
| 22 | TPP-76868 | RASQGISNYLA |
| | LCDR1 | |
| 23 | TPP-76868 | AAQIPGS |
| | LCDR2 | |
| 24 | TPP-76868 | LQHYSYPLT |
| | LCDR3 | |
| 25 | TPP-77658 | EVQLVESGGGLVQPGGSLRLSCAASGFSFGDFDMRWFRQAPGKGLEVWGTIK |
| | Heavy Chain | SRAYLEATEFAASVEGRFTISRDDAKNSAYLQMNSLRAEDTAVYYCTRDAYSS |
| | fused to H10 | GLLDYWGQGTLVTVSSASTKGPSVFPLAPSSKSTSGGTAALGCLVKDYFPEPV |
| | p35; | TVSWNSGALTSGVHTFPAVLQSSGLYSLSSVVTVPSSSLGTQTYICNVNHKPSN |
| | component of | TKVDKKVEPKSCDKTHTCPPCPAPEAAGAPSVFLFPPKPKDTLMISRTPEVTCV |
| | H10658 | VVDVSHEDPEVKFNWYVDGVEVHNAKTKPREEQYNSTYRVVSVLTVLHQDWL |
| | fusion | NGKEYKCKVSNKALPAPIEKTISKAKGQPREPQVCTLPPSREEMTKNQVSLWC |
| | | LVKGFYPSDIAVEWESNGQPENNYKTTPPVLDSDGSFFLYSKLTVDKSRWQQG |
| | | NVFSCSVMHEALHNHYTQKSLSLSPGSGGGGSGGGGGGGGRNLPVATPDP |
| | | GMFPCLHHSQNLLRAVSNMLQKARQTLEFYPCTSEEIDHEDITKDKTSTVEACL |
| | | PLELTKNESCLNSRETSFITNGSCLASRKTSFMMALCLSSIYEDLKMYQVEFKT |
| | | MNAKLLMDPKRQIFLDQNMLAVIDELMQALNFNSETVPQKSSLEEPDFAKTKIKL |
| | | CILLHAFRIRAVTIDRVMSYLNAS |
| 26 | TPP-76868 | EVQLVESGGGLVQPGGSLRLSCAASGFSFGDFDMRWFRQAPGKGLEWWGLIK |
| | Heavy Chain | SRAYLEATEFAASVEGRFTISRDDAKNSAYLQMNSLRAEDTAVYYCTRDSYSS |
| | fused to H10 | GLLDYWGQGTLVTVSSASTKGPSVFPLAPSSKSTSGGTAALGCLVKDYFPEPV |
| | p35; | TVSWNSGALTSGVHTFPAVLQSSGLYSLSSVVTVPSSSLGTQTYICNVNHKPSN |
| | component of | TKVDKKVEPKSCDKTHTCPPCPAPEAAGAPSVFLFPPKPKDTLMISRTPEVTCV |
| | H10868 | VVDVSHEDPEVKFNWYVDGVEVHNAKTKPREEQYNSTYRVVSVLTVLHQDWL |
| | fusion | NGKEYKCKVSNKALPAPIEKTISKAKGQPREPQVCTLPPSREEMTKNQVSLWC |
| | | LVKGFYPSDIAVEWESNGQPENNYKTTPPVLDSDGSFFLYSKLTVDKSRWQQG |
| | | NVFSCSVMHEALHNHYTQKSLSLSPGSGGGGSGGGGSGGGGRNLPVATPDP |
| | | GMFPCLHHSQNLLRAVSNMLQKARQTLEFYPCTSEEIDHEDITKDKTSTVEACL |
| | | PLELTKNESCLNSRETSFITNGSCLASRKTSFMMALCLSSIYEDLKMYQVEFKT |
| | | MNAKLLMDPKRQIFLDQNMLAVIDELMQALNFNSETVPQKSSLEEPDFAKTKIKL |
| | | CILLHAFRIRAVTIDRVMSYLNAS |
| 27 | Linker | SGGGGSGGGGSGGGG |
| 28 | Full length | MCPARSLLLVATLVLLDHLSLARNLPVATPDPGMFPCLHHSQNLLRAVSNMLQK |
| | human IL-12 | ARQTLEFYPCTSEEIDHEDITKDKTSTVEACLPLELTKNESCLNSRETSFITNGSC |
| | p35, | LASRKTSFMMALCLSSIYEDLKMYQVEFKTMNAKLLMDPKRQIFLDQNMLAVID |
| | including | ELMQALNFNSETVPQKSSLEEPDFYKTKIKLCILLHAFRIRAVTIDRVMSYLNAS |
| | signal | |
| | peptide | |
| 29 | Full length | MCHQQLVISWFSLVFLASPLVAIWELKKDVYVVELDWYPDAPGEMVVLTCDTP |
| | human IL-12 | EEDGITWTLDQSSEVLGSGKTLTIQVKEFGDAGQYTCHKGGEVLSHSLLLLHKK |
| | p40, | EDGIWSTDILKDQKEPKNKTFLRCEAKNYSGRFTCWWLTTISTDLTFSVKSSRG |
| | including | SSDPQGVTCGAATLSAERVRGDNKEYEYSVECQEDSACPAAEESLPIEVMVDA |
| | signal | VHKLKYENYTSSFFIRDIIKPDPPKNLQLKPLKNSRQVEVSWEYPDTWSTPHSY |
| | peptide | FSLTFCVQVQGKSKREKKDRVFTDKTSATVICRKNASISVRAQDRYYSSSWSE |
| | | WASVPCS |
| 30 | Portion of | KSKREKK |
| | p40 mutated | |
| | to GGG in | |
| | H10 mutein | |
| 31 | Mouse IL12 | MWELEKDVYVVEVDWTPDAPGETVNLTCDTPEEDDITWTSDQRHGVIGSGKTL |
| | mutein | TITVKEFLDAGQYTCHKGGETLSHSHLLLHKKENGIWSTEILKNFKNKTFLKCEA |
| | surrogate for | PNYSGRFTCSWLVQRNMDLKFNIKSSSSSPDSRAVTCGMASLSAEKVTLDQRD |
| | H10, | YEKYSVSCQEDVTCPTAEETLPIELALEARQQNKYENYSTSFFIRDIIKPDPPKNL |
| | containing | QMKPLKNSQVEVSWEYPDSWSTPHSYFSLKFFVGGGEKMKETEEGCNQKGA |
| | linked p40 | FLVEKTSTEVQCKGGNVCVQAQDRYYNSSCSKWACVPCRVRSGGGGGGSRV |
| | and p35 | IPVSGPARCLSQSRNLLKTTDDMVKTAREKLEFYPCTAEDIDHEDITRDQTSTLK |
| | | TCLPLELHKNESCLATRETSSTTRGSCLPPQKTSLMMTLCLGSIYEDLKMYQTE |
| | | FQAINAALQNHNHQQIILDKGMLVAIDELMQSLNHNGETLRQKPPVGEADPYRV |
| | | KMKLCILLHAFSTRVVTINRVMGYLSSA |
| 32 | Linker | GGGGGGS |
| | connecting | |
| | p40 and p35 | |
| | in mouse | |
| | surrogate | |
| | IL 12 mutein | |
| 33 | VH TPP- | EVQLVESGGGLVQPGGSLRLSCAASGFSFGDFDMRWFRQAPGKGLEWVGLIK |
| | 68807 | SKAYRYATEFAASVEGRFTISRDDAKNSAYLQMNSLRAEDTAVYYCTRDSYSS |
| | | GLLDYWGQGTLVTVSS |
| 34 | VL TPP- | DIQMTQSPSSLSASVGDRVTITCRASQGISNYLAWFQQKPGKAPKRLIYAASIPG |
| | 68807 | SGVPSRFSGSGSGTDFTLTISSLQPEDFATYYCLQHKSYPLTFGGGTKVEIK |
| 35 | VH TPP- | GFSFGDF |
| | 77658, TPP- | |
| | 76868, and | |
| | TPP-68807 | |
| | CDR1 | |
| | Chothia | |
| 36 | VH TPP- | DFDMR |
| | 77658, TPP- | |
| | 76868, and | |
| | TPP-68807 | |
| | CDR1 Kabat | |
| 37 | VH TPP- | KSRAYLEA |
| | 77658 and | |
| | TPP-76868 | |
| | CDR2 | |
| | Chothia | |
| 38 | VH TPP- | KSKAYRYA |
| | 68807 CDR2 | |
| | Chothia | |
| 39 | VH TPP- | LIKSKAYRYATEFAASVEG |
| | 68807 CDR2 | |
| | Kabat and | |
| | Extended | |
| 40 | VL TPP- | AASIPGS |
| | 68807 CDR2 | |
| 41 | VL TPP- | LQHKSYPLT |
| | 68807 CDR3 | |
| 42 | TPP-68807 | EVQLVESGGGLVQPGGSLRLSCAASGFSFGDFDMRWFRQAPGKGLEWVGLIK |
| | Heavy Chain | SKAYRYATEFAASVEGRFTISRDDAKNSAYLQMNSLRAEDTAVYYCTRDSYSS |
| | | GLLDYWGQGTLVTVSSASTKGPSVFPLAPSSKSTSGGTAALGCLVKDYFPEPV |
| | | TVSWNSGALTSGVHTFPAVLQSSGLYSLSSVVTVPSSSLGTQTYICNVNHKPSN |
| | | TKVDKKVEPKSCDKTHTCPPCPAPEAAGAPSVFLFPPKPKDTLMISRTPEVTCV |
| | | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPCREEMTKNQVSLSCA |
| | | VKGFYPSDIAVEWESNGQPENNYKTTPPVLDSDGSFFLVSKLTVDKSRWQQG |
| | | NVFSCSVMHEALHNHYTQKSLSLSPGK |
| 43 | TPP-68807 | DIQMTQSPSSLSASVGDRVTITCRASQGISNYLAWFQQKPGKAPKRLIYAASIPG |
| | Light Chain | SGVPSRFSGSGSGTDFTLTISSLQPEDFATYYCLQHKSYPLTFGGGTKVEIKRT |
| | | VAAPSVFIFPPSDEQLKSGTASVVQLLNNFYPREAKVQWKVDNALQSGNSQES |
| | | VTEQDSKDSTYSLSSTLTLSKADYEKHKVYACEVTHQGLSSPVTKSFNRGEC |
| 44 | H10 p35 | CGGAACCTCCCCGTCGCAACTCCTGACCCGGGGATGTTCCCTTGTCTGCAC |
| | nucleotide | CATAGCCAGAACCTGTTGAGAGCCGTGTCCAACATGCTCCAGAAAGCCCGG |
| | | CAGACTCTTGAGTTTTACCCATGCACCTCCGAAGAAATCGACCATGAAGATA |
| | | TTACCAAAGACAAGACCTCCACTGTGGAAGCGTGCTTGCCACTTGAGCTGA |
| | | CTAAGAACGAAAGCTGCCTGAACTCCCGGGAGACTTCTTTCATCACCAATG |
| | | GTTCCTGCCTTGCGTCCCGCAAGACCTCTTTTATGATGGCCCTGTGCCTGT |
| | | CAAGCATATACGAAGATCTGAAGATGTACCAAGTCGAGTTCAAGACCATGAA |
| | | TGCGAAGCTCCTTATGGACCCTAAGCGGCAGATCTTCCTGGATCAGAACAT |
| | | GCTGGCTGTGATCGACGAGCTGATGCAGGCTCTGAACTTCAACAGCGAGAC |
| | | AGTGCCGCAAAAGTCCAGCCTGGAAGAACCQGACTTCGCCAAGACCAAGAT |
| | | CAAGCTGTGCATTCTGCTGCACGCATTCAGGATCAGGGCAGTCACTATTGA |
| | | CAGAGTGATGTCCTACCTGAACGCCAGC |
| 45 | H10 p40 | ATCTGGGAACTGAAGAAAGATGTGTACGTGGTCGAACTTGACTGGTACCCCGAT |
| | nucleotide; | GCGCCTGGAGAGATGGTCGTGCTGACTTGCGATACGCCTGAGGAAGATGGAAT |
| | also H10 p40 | CACTTGGACTCTCGACCAGTCGTCCGAAGTCCTCGGGTCGGGAAAGACCCTGA |
| | from H10658 | CAATCCAGGTCAAGGAGTTCGGGGACGCCGGACAGTACACTTGCCACAAGGGC |
| | | GGAGAAGTGCTGTCACACTCCCTGCTGCTCCTCCACAAGAAGGAAGATGGCATC |
| | | TGGTCCACTCTGATCCTCAAGGACCAGAAGGAGCCGAAGAACAAGACTTTCCTG |
| | | CGCTGCGAGGCCAAGAACTACTCCGGACGGTTCACGTGTTGGTGGCTGACCAC |
| | | CATTAGCACCGACCTGACCTTCTCCGTGAAGTCCAGCCGGGGGAGCAGCGACC |
| | | CGCAGGGAGTGACCTGTGGCGCCGCGACCCTCTCCGCTGAGCGCGTGGGGGG |
| | | AGACAACAAGGAATATGAGTACAGCGTGGAGTGTCAGGAAGATTCCGCCTGTQC |
| | | TGCTGCCGAAGAGTCGCTGCCAATTGAAGTGATGGTCGATGCCGTGCATAAGTT |
| | | GAAATACGAGAACTACACCTCGTCGTTCTTCATCCGGGACATCATTAAGCCCGA |
| | | CCCGCCCAAGAACTTGCAGCTGAAGCCCCTGAAGAACTCGAGACAGGTCGAAG |
| | | TGTCCTGGGAGTATCCCGACACCTGGTCCACCCCCCATTCGTACTTCTCGCTGA |
| | | CTTTCTGTGTGCAAGTGCAGGGTGGCGGGGGGGACAGGGTGTTCACCGATAAG |
| | | ACCTCAGCCACTGTGATTTGCCGCAAGAACGCGTCAATTTCAGTCAGGGCCCAG |
| | | GATCGGTATTACTCCTCGTCATGGTCCGAATGGGCCTCCGTGCCCTGCTCG |
| 46 | TPP-77658 | GAGGTGCAACTGGTGGAAAGCGGAGGAGGCCTGGTGCAGCCCGGQGGATCTC |
| | VH NT | TGCGGCTGTCTTGTGCCGCTTCTGGCTTCAGCTTCGGCGACTTCGACATGCGGT |
| | | GGTTTAGACAGGCCCCTGGCAAGGGCCTCGAGTGGGTGGGCACCATCAAAAGC |
| | | AGAGCTTATCTGGAAGCCACCGAGTTCGCCGCCAGCGTGGAAGGCAGATTCAC |
| | | CATCAGCCGGGACGACGCCAAGAACTQCGQCTACCTGCAGATGAACAGCCTGA |
| | | GAGCCGAGGACACCGCCGTGTACTACTGCACCAGAGATGCCTACAGCAGGGGC |
| | | CTGCTGGATTACTGGGGCCAGGGCACACTGGTCACAGTGTCCAGC |
| 47 | TPP-77658 | GATATCCAGATGACCCAGAGCCCTAGCTCTCTGAGCGCCAGCGTGGGCGACAG |
| | VL NT | AGTGACCATCACCTGTAGAGCCTCTCAGGGCATCTCCAACTACCTGGCTTGGTT |
| | | CCAGCAGAAACCTGGCAAGGCCCCTAAGCGGCTGATCTACGCCGCTCAGATCC |
| | | CAGGCAGCGGCGTCCCCAGCAGATTCAGCGGCTCCGGCTCTGGCACCGACTTC |
| | | ACCCTGACCATTAGCAGCCTGCAACCTGAGGACTTTGCCACATATTACTGCCTG |
| | | CAGCACTACAGCTACCCCCTGACATTCGGCGGAGGAACAAAGGTGGAAATCAA |
| | | G |
| 48 | H10658 | GAGGTGCAACTGGTGGAAAGCGGAGGAGGCCTGGTGCAGCCCGGQGGATCTC |
| | heavy chain | TGCGGCTGTCTTGTGCCGQTTCTGGCTTCAGCTTCGGCGACTTCGACATGCGGT |
| | NT | GGTTTAGACAGGCCCCTGGCAAGGGCCTCGAGTGGGTGGGCACCATCAAAAGC |
| | | AGAGCTTATCTGGAAGCCACCGAGTTCGCCGCCAGCGTGGAAGGCAGATTCAC |
| | | CATCAGCCGGGACGACGCCAAGAACTCCGCCTACCTGCAGATGAACAGCQTGA |
| | | GAGCCGAGGACACCGCCGTGTACTACTGCACCAGAGATGCCTACAGCAGCGGC |
| | | CTGQTGGATTACTGGGGCCAGGGCACACTGGTCACAGTGTCCAGCGCGTCGAC |
| | | CAAGGGCCCATCGGTCTTCCCCCTGGCACCCTCCTCCAAGAGCACCTCTGGGG |
| | | GCACAGCGGCCCTGGGCTGCCTGGTCAAGGACTACTTCCCCGAACCGGTGACG |
| | | GTGTCGTGGAACTCAGGCGCCCTGACCAGCGGCGTGCACACCTTCCCGGCTGT |
| | | CCTACAGTCCTCAGGACTCTACTCCCTCAGCAGCGTGGTGACCGTGCCCTCCA |
| | | GCAGCTTGGGCACCCAGACCTACATCTGCAACGTGAATCACAAGCCCAGCAACA |
| | | CCAAGGTGGACAAGAAAGTTGAGCCCAAATCTTGTGACAAAACTCACACATGCC |
| | | CACCGTGCCCAGCACCTGAAGCCGCTGGGGCACCGTCAGTCTTCCTCTTCCCC |
| | | CCAAAACCCAAGGACACCCTCATGATCTCCCGGACCCCTGAGGTCACATGCGT |
| | | GGTGGTGGACGTGAGCCACGAAGACCCTGAGGTCAAGTTCAACTGGTACGTGG |
| | | ACGGCGTGGAGGTGCATAATGCCAAGACAAAGCCGCGGGAGGAGCAGTACAAC |
| | | AGCACGTACCGTGTGGTCAGCGTCCTCACCGTCCTGCACCAGGACTGGQTGAA |
| | | TGGCAAGGAGTACAAGTGCAAGGTCTCCAACAAAGCCCTCCCAGCCCCCATCG |
| | | AGAAAACCATCTCCAAAGCCAAAGGGCAGCCCCGAGAACCACAGGTGTACACC |
| | | CTGCCCCCATGCCGGGAGGAGATGACCAAGAACCAGGTCAGCCTGTCCTGCGC |
| | | GGTCAAAGGCTTCTATCCCAGCGACATCGCCGTGGAGTGGGAGAGCAATGGGC |
| | | AGCCGGAGAACAACTACAAGACCACGCCTCCCGTGQTGGACTCCGACGGCTCQ |
| | | TTCTTCCTCGTTAGCAAGCTCACCGTGGACAAGAGCAGGTGGCAGCAGGGGAA |
| | | CGTCTTCTCATGCTCCGTGATGCATGAGGCTCTGCACAACCACTACACGCAGAA |
| | | GAGCCTCTCCCTGTCCCCCGGAAAA |
| 49 | H10658 | GAGGTGCAACTGGTGGAAAGCGGAGGAGGCCTGGTGCAGCCCGGCGGATCTC |
| | heavy chain | TGCGGCTGTCTTGTGCCGCTTCTGGCTTCAGCTTCGGCGACTTCGACATGCGGT |
| | fused to p35 | GGTTTAGACAGGCCCCTGGCAAGGGCCTCGAGTGGGTGGGCACCATCAAAAGC |
| | NT | AGAGCTTATCTGGAAGCCACCGAGTTCGCCGCCAGCGTGGAAGGCAGATTCAC |
| | | CATCAGCCGGGACGACGCCAAGAACTCCGCCTACCTGCAGATGAACAGCCTGA |
| | | GAGCCGAGGACACCGCCGTGTACTACTGCACCAGAGATGCCTACAGCAGGGGC |
| | | CTGCTGGATTACTGGGGCCAGGGCACACTGGTCACAGTGTCCAGCGCGTCGAC |
| | | CAAGGGCCCATCGGTCTTCCCCCTGGCACCCTCCTCCAAGAGCACCTCTGGGG |
| | | GCACAGCGGCCCTGGGCTGCCTGGTCAAGGACTACTTCCCCGAACCGGTGACG |
| | | GTGTCGTGGAACTCAGGCGCCCTGACCAGCGGCGTGCACACCTTCCCGGCTGT |
| | | CCTACAGTCCTCAGGACTCTACTCCCTCAGCAGCGTGGTGACCGTGCCCTQCA |
| | | GCAGCTTGGGCACCCAGACCTACATCTGCAACGTGAATCACAAGCCCAGCAACA |
| | | CCAAGGTGGACAAGAAAGTTGAGCCCAAATCTTGTGACAAAACTCACACATGCC |
| | | CACCGTGCCCAGCACCTGAAGCCGCTGGGGCACCGTCAGTQTTCCTCTTCCCC |
| | | CCAAAACCCAAGGACACCQTCATGATCTCCCGGACCCCTGAGGTCACATGCGT |
| | | GGTGGTGGACGTGAGCCACGAAGACCCTGAGGTCAAGTTCAACTGGTACGTGG |
| | | ACGGCGTGGAGGTGCATAATGCCAAGACAAAGCCGCGGGAGGAGCAGTACAAC |
| | | AGCACGTACCGTGTGGTCAGCGTCCTCACCGTCCTGCACCAGGACTGGCTGAA |
| | | TGGCAAGGAGTACAAGTGCAAGGTCTCCAACAAAGCCCTQCCAGCCCCCATCG |
| | | AGAAAACCATCTCCAAAGCCAAAGGGCAGCCCCGAGAACCACAGGTGTGCACC |
| | | CTGCCCCCATCCCGGGAGGAGATGACCAAGAACCAGGTCAGCCTGTGGTGCCT |
| | | GGTCAAAGGCTTCTATCCCAGCGACATCGCCGTGGAGTGGGAGAGCAATGGGC |
| | | AGCCGGAGAACAACTACAAGACCACGCCTCCCGTGCTGGACTCCGACGGCTCC |
| | | TTCTTCCTCTATAGCAAGCTCACCGTGGACAAGAGCAGGTGGCAGCAGGGGAA |
| | | CGTCTTCTCATGCTCCGTGATGCATGAGGCTCTGCACAACCACTACACGCAGAA |
| | | GAGCCTCTCCCTGTCCCCCGGAAGCGGTGGAGGAGGTTCCGGTGGCGGAGGT |
| | | TCAGGTGGCGGAGGGGGGAACCTCCCCGTCGCAACTCCTGACCCGGGGATGTT |
| | | CCCTTGTCTGCACCATAGCCAGAACCTGTTGAGAGCCGTGTCCAACATGQTCCA |
| | | GAAAGCCCGGCAGACTCTTGAGTTTTACCCATGCACCTCCGAAGAAATCGACCA |
| | | TGAAGATATTACCAAAGACAAGACCTCCACTGTGGAAGCGTGCTTGCCACTTGA |
| | | GCTGACTAAGAACGAAAGCTGCCTGAACTCCCGGGAGACTTCTTTCATCACCAA |
| | | TGGTTCCTGCCTTGCGTCCCGCAAGACCTCTTTTATGATGGCCCTGTGCCTGTC |
| | | AAGCATATACGAAGATCTGAAGATGTACCAAGTCGAGTTCAAGACCATGAATGC |
| | | GAAGCTCCTTATGGACCCTAAGCGGCAGATQTTCCTGGATCAGAACATGCTGGC |
| | | TGTGATCGACGAGCTGATGCAGGCTCTGAACTTCAACAGCGAGACAGTGCCGC |
| | | AAAAGTCCAGCCTGGAAGAACCCGACTTCGCCAAGACCAAGATCAAGCTGTGCA |
| | | TTCTGCTGCACGCATTCAGGATCAGGGCAGTCACTATTGACAGAGTGATGTQCT |
| | | ACCTGAACGCCAGC |
| 50 | H10658 light | GATATCCAGATGACCCAGAGCCCTAGCTCTCTGAGCGCCAGCGTGGGCGACAG |
| | chain NT | AGTGACCATCACCTGTAGAGCCTCTCAGGGCATCTCCAACTACCTGGCTTGGTT |
| | | CCAGCAGAAACCTGGCAAGGCCCCTAAGCGGCTGATCTACGCCGCTCAGATCC |
| | | CAGGCAGCGGCGTCCCCAGCAGATTCAGCGGCTCCGGCTCTGGCACCGACTTC |
| | | ACCCTGACCATTAGCAGCCTGCAACCTGAGGACTTTGCCACATATTACTGCCTG |
| | | CAGCACTACAGCTACCCCCTGACATTQGGCGGAGGAACAAAGGTGGAAATCAA |
| | | GCGTACGGTGGCTGCACCATCTGTCTTCATCTTCCCGCCATCTGATGAGCAGTT |
| | | GAAATCTGGAACTGCCTCTGTTGTGTGCCTGCTGAATAACTTCTATQCCAGAGA |
| | | GGCCAAAGTACAGTGGAAGGTGGATAACGCCCTCCAATCGGGTAACTCCCAGG |
| | | AGAGTGTCACAGAGCAGGACAGCAAGGACAGCACCTACAGCCTCAGCAGCACC |
| | | CTGACGCTGAGCAAAGCAGACTACGAGAAACACAAAGTCTACGCCTGCGAAGTC |
| | | ACCCATCAGGGCCTGAGCTCGCCCGTCACAAAGAGCTTCAACAGGGGAGAGTG |
| | | T |
| 51 | TPP-77658 | EVQLVESGGGLVQPGGSLRLSCAASGFSFGDFDMRWFRQAPGKGLEWWVGTIK |
| | Heavy Chain | SRAYLEATEFAASVEGRFTISRDDAKNSAYLQMNSLRAEDTAVYYCTRDAYSS |
| | without hole | GLLDYWGQGTLVTVSSASTKGPSVFPLAPSSKSTSGGTAALGCLVKDYFPEPV |
| | mutations | TVSWNSGALTSGVHTFPAVLQSSGLYSLSSVVTVPSSSLGTQTYICNVNHKPSN |
| | | TKVDKKVEPKSCDKTHTCPPCPAPEAAGAPSVFLFPPKPKDTLMISRTPEVTCV |
| | | WVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |
| 52 | TPP-77658 | EVQLVESGGGLVQPGGSLRLSCAASGFSFGDFDMRWFRQAPGKGLEWWVGTIK |
| | Heavy Chain | SRAYLEATEFAASVEGRFTISRDDAKNSAYLQMNSLRAEDTAVYYCTRDAYSS |
| | without hole | GLLDYWGQGTLVTVSSASTKGPSVFPLAPSSKSTSGGTAALGCLVKDYFPEPV |
| | or effector | TVSWNSGALTSGVHTFPAVLQSSGLYSLSSVVTVPSSSLGTQTYICNVNHKPSN |
| | null | TKVDKKVEPKSCDKTHTCPPCPAPELLGGPSVFLFPPKPKDTLMISRTPEVTCV |
| | mutations | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |
| 53 | TPP-76868 | EVQLVESGGGLVQPGGSLRLSCAASGFSFGDFDMRWFRQAPGKGLEWVGLIK |
| | Heavy Chain | SRAYLEATEFAASVEGRFTISRDDAKNSAYLQMNSLRAEDTAVYYCTRDSYSS |
| | without hole | GLLDYWGQGTLVTVSSASTKGPSVFPLAPSSKSTSGGTAALGCLVKDYFPEPV |
| | mutations | TVSWNSGALTSGVHTFPAVLQSSGLYSLSSVVTVPSSSLGTQTYICNVNHKPSN |
| | | TKVDKKVEPKSCDKTHTCPPCPAPEAAGAPSVFLFPPKPKDTLMISRTPEVTCV |
| | | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |
| 54 | TPP-76868 | EVQLVESGGGLVQPGGSLRLSCAASGFSFGDFDMRWFRQAPGKGLEWVGLIK |
| | Heavy Chain | SRAYLEATEFAASVEGRFTISRDDAKNSAYLQMNSLRAEDTAVYYCTRDSYSS |
| | without hole | GLLDYWGQGTLVTVSSASTKGPSVFPLAPSSKSTSGGTAALGCLVKDYFPEPV |
| | or effector | TVSWNSGALTSGVHTFPAVLQSSGLYSLSSVVTVPSSSLGTQTYICNVNHKPSN |
| | null | TKVDKKVEPKSCDKTHTCPPCPAPELLGGPSVFLFPPKPKDTLMISRTPEVTCV |
| | mutations | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |
| 55 | TPP-68807 | EVQLVESGGGLVQPGGSLRLSCAASGFSFGDFDMRWFRQAPGKGLEWVGLIK |
| | Heavy Chain | SKAYRYATEFAASVEGRFTISRDDAKNSAYLQMNSLRAEDTAVYYCTRDSYSS |
| | without hole | GLLDYWGQGTLVTVSSASTKGPSVFPLAPSSKSTSGGTAALGCLVKDYFPEPV |
| | mutations | TVSWNSGALTSGVHTFPAVLQSSGLYSLSSVVTVPSSSLGTQTYICNVNHKPSN |
| | | TKVDKKVEPKSCDKTHTCPPCPAPEAAGAPSVFLFPPKPKDTLMISRTPEVTCV |
| | | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |
| 56 | TPP-68807 | EVQLVESGGGLVQPGGSLRLSCAASGFSFGDFDMRWFRQAPGKGLEWVGLIK |
| | Heavy Chain | SKAYRYATEFAASVEGRFTISRDDAKNSAYLQMNSLRAEDTAVYYCTRDSYSS |
| | without hole | GLLDYWGQGTLVTVSSASTKGPSVFPLAPSSKSTSGGTAALGCLVKDYFPEPV |
| | or effector | TVSWNSGALTSGVHTFPAVLQSSGLYSLSSVVTVPSSSLGTQTYICNVNHKPSN |
| | null | TKVDKKVEPKSCDKTHTCPPCPAPELLGGPSVFLFPPKPKDTLMISRTPEVTCV |
| | mutations | VVDVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### EXAMPLES

In order that this invention may be better understood, the following examples are set forth. These examples are for purposes of illustration only and are not to be construed as limiting the scope of the invention in any manner.

### Example 1: IL-12 Variants

The objective of this experiment was to generate human IL-12 variants (also referred to as IL-12 mutated proteins or “muteins”) having attenuated IL-12 activity relative to wild-type human IL-12.

A set of IL-12 variants were designed with mutations engineered into one or both of the p35 and the p40 subunit of human IL-12. The IL-12 variants are described in Table 10. In Table 10, the mutation positions are numbered based on the amino acid sequence of the mature human p35 and p40 proteins (SEQ ID NO: 1 and SEQ ID NO: 2, respectively). As shown in Table 10, the mutations were in one or more of positions F39, I52, and Y167 of the p35 subunit, and one or more of positions 093 and K85 of the p40 subunit. These amino acid positions are predicted to be located at the interface between IL12 and the IL12 receptor.

TABLE 10

| | | | | | | | EC₅₀ (ug/ml) |
|---|---|---|---|---|---|---|---|
| | | | | | | EC₅₀ (ug/ml) | hIL12R+ |
| | P35 | | | P40 | | hIL12R+ | PD1+ |
| Mutein | F39 | I52 | Y167 | D93 | K85 | cells | cells |
| H1 | R | | | | | 1.7 | 0.6 |
| H2 | | E | | | | 0.65 | 0.007 |
| H3 | | | A | | | 12.8 | 0.34 |
| H4 | | | | L | | 46.7 | 0.19 |
| H5 | | | | | E | 12.6 | 0.03 |
| H6 | R | | | L | | 138 | 13.9 |
| H7 | R | | | | E | 4 | 0.67 |
| H8 | | E | | L | | 221.8 | 331 |
| H9 | | E | | | E | 8.9 | 0.16 |
| H10 | | | A | L | | Undetermined; >3000 | 686 |
| H11 | | | A | | E | | |
| H12 | R | E | A | L | | | |
| H13 | R | E | A | | E | 72 | 8.8 |
| H14 | R | E | A | L | E | No response | No response |
| H15 | | R | | | | | |
| H16 | | R | | L | | | |
| H17 | | R | | L | E | 5.8 | 0.07 |
| H18 | | R | A | | | 46.6 | 0.85 |
| H19 | | R | A | L | E | | |
| H20 | R | R | | L | | 475.8 | Doesn't |
| | | | | | | | saturate |
| H21 | A | R | | L | | | |
| H22 | A | R | | L | E | 149 | 52 |
| H23 | A | H | | L | E | | |
| H24 | | H | A | L | E | 562 | 6.6 |
| H25 | A | H | A | L | E | | |
| H30 | R | | A | L | | | |
| H31 | | R | A | L | | | |
| H32 | R | R | A | | | | |
| WT IL-12 | | | | | | ~0.1 | 0.03 |

Fusion proteins were prepared in which the different IL-12 muteins shown in Table 10 were linked to a platform anti-human PD1 antibody. The activity of the different IL-12 mutein/anti-PD1 fusion proteins was then assessed.

First, the activity of the IL-12 mutein/anti-PD1 fusion proteins was assessed using an IL-12 receptor positive (IL12R+), human PD1-negative pSTAT4 reporter cell line. The half maximal effective concentration (EC₅₀) for the fusion proteins with this cell line (assessing STAT4 phosphorylation) is shown in the column “EC50 hIL12R+ cells” of Table 10. The EC₅₀ is provided in micrograms per milliliter (ug/ml). A lower EC₅₀ value indicates greater activity than a higher EC₅₀ value.

Next, the activity of the IL-12 mutein/anti-PD1 fusion proteins was assessed using an IL-12 receptor positive (IL12R+), human PD1-positive pSTAT4 reporter cell line. The half maximal effective concentration (EC₅₀) for the fusion proteins with this cell line (assessing STAT4 phosphorylation) is shown in the column “EC50 hIL12R+ PD1+ cells” of Table 10. This data provides information regarding the P01-induced rescued IL-12 mutein activity of each fusion protein (e.g. by comparing the activity of each fusion protein between P1DI-negative and PD1-positive cell lines).

As shown in Table 10, multiple IL-12 mutein/anti-PD1 fusion proteins have low activity in hIL12R+, PD1-negative cells, but greater activity in hIL12R+, PD1-positive cells (e.g. mutein H10). Thus, these fusion proteins have targeted IL-12 activity on PD1-positive cells.

As further shown in Table 10, the mutein “H10” has an undetermined but low activity (EC₅₀ greater than 3000 ug/ml; the highest concentration tested) on PD1-negative cells, but greater activity (EC₅₀ 686 ug/ml) on PD1-positive cells.

The H10 mutein was selected for further development based on multiple favorable characteristics identified in these assays. First, as noted above, the H10 mutein linked to an anti-PD1 antibody has low activity on PD1-negative cells. The lack of activity on PD1-negative cells potentially limits the number of cells that can be effectively stimulated by the H10 IL-12 mutein, and therefore potentially reduces toxicity that can be associated with IL-12 activity (e.g. caused by IL-12-mediated overstimulation of the immune response). Second, while the H10 mutein linked to an anti-PD1 antibody has low activity on PD1-negative cells, the H10 IL-12 mutein/anti-PD1 fusion protein still has detectable activity on PD1-positive cells. Therefore, the H10 IL-12 mutein/anti-PD1 fusion protein is hypothesized to still have activity in environments that are enriched with PD1-positive cells, such as the tumor microenvironment (TME). Third, assessment of biophysical properties of the H10 mutein (e.g. stability and predicted immunogenicity) indicated that the H10 mutein had more desirable molecular properties than other IL12-muteins that also had biased activity toward PD1-positive cells as compared to PD1-negative cells when linked to an anti-PD1 antibody.

The H10 IL12 mutein contains the mature p35 amino acid sequence of SEQ ID NO: 3 and the mature p40 amino acid sequence of SEQ ID NO: 4. As shown in SEQ ID NO: 3 below, the H10 p35 subunit contains the mutation Y167A (underlined), and as shown in SEQ ID NO: 4 below, the H10 p40 subunit contains the mutation D93L (underlined) and also the heparin-binding site mutations GGG (underlined). For the heparin binding site mutations, the amino acid sequence KSKREKK (SEQ ID NO: 30) in the wild-type IL-12p40 sequence (amino acids 258-264 in SEQ ID NO: 4) is mutated and truncated down to the sequence “GGG”. Wild-type IL-12 is known to bind to heparin and heparan sulfate (Hasan M, et. al. J Immunol. 1999 Jan. 15; 162(2): 1064-70); the mutation of the sequence KSKREKK (SEQ ID NO: 30) in wild-type IL-12p40 to GGG reduces the affinity of IL-12p40 to heparin and heparan sulfate. Binding of IL-12 to heparin is hypothesized to enhance IL-12 function (e.g. by retaining IL-12 close to the sites of secretion, serving to maintain high local cytokine concentration). In addition, the heparin binding site in IL-12 is protease sensitive; accordingly, removing the site reduces the protease-sensitivity of the H10 mutein.

H10 Mutein p35:
(SEQ ID NO: 3)
RNLPVATPDPGMFPCLHHSQNLLRAVSNMLQKARQTLEFYPCTSEEIDHE
DITKDKTSTVEACLPLELTKNESCLNSRETSFITNGSCLASRKTSFMMAL
CLSSIYEDLKMYQVEFKTMNAKLLMDPKRQIFLDQNMLAVIDELMQALNF
NSETVPQKSSLEEPDFAKTKIKLCILLHAFRIRAVTIDRVMSYLNAS
H10 Mutein p40:
(SEQ ID NO: 4)
IWELKKDVYVVELDWYPDAPGEMVVLTCDTPEEDGITWTLDQSSEVLGSG
KTLTIQVKEFGDAGQYTCHKGGEVLSHSLLLLHKKEDGIWSTLILKDQKE
PKNKTFLRCEAKNYSGRFTCWWLTTISTDLTFSVKSSRGSSDPQGVTCGA
ATLSAERVRGDNKEYEYSVECQEDSACPAAEESLPIEVMVDAVHKLKYEN
YTSSFFIRDIIKPDPPKNLQLKPLKNSRQVEVSWEYPDTWSTPHSYFSLT
FCVQVQGGGGDRVFTDKTSATVICRKNASISVRAQDRYYSSSWSEWASVP
CS

### Example 2: Non-Blocking Anti-PD1 Antibodies

A multi-step process was used to generate and select new non-blocking anti-human PD1 (hPD1) antibodies. The anti-hPD1 antibodies generated include the clones GBT-PD1-0009, GBT-PD1-0013, and GBT-PD1-0017.

Non-blocking anti-hPD1 antibodies are antibodies that do not bind to the same epitope on hPD1 as bound by the hPD1 ligand, human PDL1 (hPDL1). Non-blocking anti-hPD1 antibodies do not prevent the binding of hPDL1 to hPD1. Additionally, non-blocking antibodies potentially do not prevent the binding to PD1 of many currently available therapeutic antagonist anti-hPD1 antibodies (e.g. pembrolizumab, cemiplimab, nivolumab, and others), which interfere with the binding of hPDL1 to hPD1.

In order to assess whether various antibodies could simultaneously bind to hPD1, sandwich assays were performed in which 2 different antibodies were incubated at the same time with hPD1. In addition, in some assays an antibody was incubated with a fusion molecule containing hPDL1 covalently linked to an antibody Fc domain (hPDL1-Fc), order to assess whether the respective antibody could bind to hPD1 at the same time as hPDL1.

Table 11 shows results from the sandwich assays. In Table 11, the corresponding position in the Table is listed with a “Y” if the paired antibodies listed in the corresponding column and row can simultaneously bind hPD1 (i.e. there isn't competition between the antibodies for binding to hPD1), or “N” if there is competition between the antibodies for binding to hPD1. As an example, as shown in Table 11, the binding of cemiplimab to hPD1 is interfered with when nivolumab, pembrolizumab, or hPDL1 are present (as noted by the “N”) in each of these positions in the table. In contrast, the binding of cemiplimab to hPD1 is not interfered with in the presence of GBT-PD1-0009, GBT-PD1-0013, or GBT-PD1-0017.

TABLE 11

| | | | | GBT- | GBT- | GBT- | |
|---|---|---|---|---|---|---|---|
| | | | | PD1- | PD1- | PD1- | hPDL1- |
| | Cemiplimab | Nivolumab | Pembrolizumab | 0009 | 0017 | 0013 | Fc |
| Cemiplimab | | N | N | Y | Y | Y | N |
| Nivolumab | N | | N | Y | Y | Y | N |
| Pembrolizumab | N | N | | Y | Y | Y | N |
| GBT-PD1-0009 | Y | Y | Y | | N | Y | Y |
| GBT-PD1-0017 | Y | Y | Y | N | | N | Y |
| GBT-PD1-0013 | Y | Y | Y | Y | N | | Y |

For each of GBT-PD1-0009, GBT-PD1-0013, or GBT-PD1-0017, the binding of the respective antibody to PD1 is not interfered with by hPDL1, cemiplimab, nivolumab, or pembrolizumab, indicating that GBT-PD1-0009, GBT-PD1-0013, and GBT-PD1-0017 do not bind to PD1 at the position bound by hPDL1, or the epitope bound by cemiplimab, nivolumab or pembrolizumab.

To validate the non-blocking nature of GBT-PD1-0013, an X-ray crystal structure was elucidated for GBT-PD1-0013 in complex with the extracellular domain (ECD) of human PD1. The crystal structure showed that GBT-PD1-0013 binds to human PD1 ECD with a 1:1 stoichiometry at a site different from that of the native ligand PD-L1, as determined by aligning the relevant complex structures.

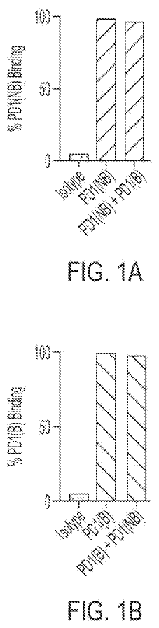

Simultaneous binding of a blocking anti-human PD1 antibody [“PD1(B)”] and the non-blocking anti-PD1 antibody GBT-PD1-0013 was evaluated by flow cytometry-based readouts using the BW5147.3 (T lymphoblast) cell line over-expressing human PD1. To enable flow cytometry-based readouts, PD1(B) and GBT-PD1-0013 were conjugated to fluorescent dyes (AlexaFluor488 and AlexaFluor647, respectively). Fixed number of cells were incubated with: i) the individual antibodies to assess maximum individual binding, or ii) a 1:1 mixture of the antibodies to assess simultaneous binding of the antibodies. An isotype control antibody was used as a negative control for binding to PD1. The results are shown in FIGS. 1A and 1B. In these FIGs, GBT-PD1-0013 is referred to as “PD1(NB)” and the blocking anti-PD1 antibody is referred to as “PD1(B)”. FIG. 1A and FIG. 1B show that GBT-PD1-0013 binding to PD1-expressing cells was only minimally impacted by the presence of PD1(B), and vice-versa. Specifically, FIG. 1A shows that the binding of GBT-PD1-0013 to PD1-expressing cells is only minimally impacted by the presence of PD1(B) (i.e. the % of PD1-expressing cells that are bound by GBT-PD1-0013 only slightly decreased when GBT-PD1-0013 and PD1(B) are co-incubated with PD1-expressing cells, as compared to when GBT-PD1-0013 alone is incubated with PD1-expressing cells). Similarly, FIG. 1B shows that that the binding of PD1(B) to PD1-expressing cells is only minimally impacted by the presence of GBT-PD1-0013.

The clone GBT-PD1-0013 was modified to generate a series of related non-blocking anti-hPD1 antibodies that have varying affinities towards hPD1, and that also have reduced predicted immunogenicity (based on lower number of predicted T cell epitopes) as compared to the parental GBT-PD1-0013 antibody. Binding characteristics of the different clones to hPD1 are provided in Table 12.

TABLE 12

| | kₐ (1/Ms) | k_{d} | K_{D} | t_{1/2} |
|---|---|---|---|---|
| GBT-PD1-0013 | 7.47E+04 | 2.65E−03 | 35 | 262 |
| (parent) | | | | |
| TPP-68807 | 7.85E+04 | 2.51E−04 | 3 | 2760 |
| TPP-76837_nd | 1.12E+05 | 3.61E−04 | 3 | 1921 |
| TPP-76852 | 1.14E+05 | 4.14E−04 | 4 | 1674 |
| TPP-76853 | 1.14E+05 | 4.37E−04 | 4 | 1587 |
| TPP-76868 | 1.35E+05 | 3.91E−04 | 3 | 1773 |
| TPP-76869 | 7.61E+04 | 4.87E−04 | 6 | 1422 |
| TPP-77658 | 1.09E+05 | 7.65E−04 | 7 | 906 |

### Example 3: IL-12 Mutein—Non-Blocking Anti-PD1 Antibody Fusion Proteins

This example describes the preparation and properties of fusion proteins that combine the IL-12 H10 mutein (Example 1) with various non-blacking anti-PD1 antibodies (Example 2).

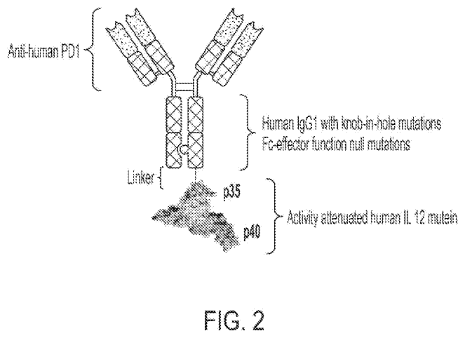

Multiple non-blocking anti-PD1 antibodies as described in Example 2 were covalently linked to the IL-12 mutein H10 via a serine-glycine linker. Specifically, the p35 subunit of the IL-12 H10 mutein was connected to the C-terminus of one heavy chain of the antibody via a linker having the amino acid sequence: SGGGGSGGGGSGGGG (SEQ ID NO: 27). (The p40 subunit of IL-12 H10 mutein associates with the antibody via its interaction with the p35 subunit.) A general schematic of the fusion proteins is shown in FIG. 2 As shown in FIG. 2, one of the heavy chains of the anti-human PD1 antibody is covalently linked to the p35 subunit of the IL-12 mutein via a linker; the p40 subunit of IL-12 associates with p35 via a disulfide bond between C74 of p35 and C177 of p40. In addition, to promote heterodimerization between 1) the antibody heavy chain that is covalently linked to p35 and 2) the antibody heavy chain that isn't linked to p35, each heavy chain has mutations in the Fc-region to form either a knob or hole structure. As depicted in FIG. 2, the antibody heavy chain that is covalently linked to p35 has mutations to form a knob and the heavy chain that isn't linked to p35 has mutations to form a hole. In addition, both heavy chains have mutations in the Fc domain to render the Fc effector null. The antibody is IgG1 subclass.

The fusion proteins were prepared with an inert Fc domain in order to eliminate potential Fc-mediated depletion and/or Fc receptor binding, and further in order to focus binding and activity of IL-12 towards PD1-positive cells.

Overall, the anti-PD1 portion of the IL-12 H10 mutein/anti-PD1 fusion protein helps to “anchor” the fusion protein to cells that are PD1-positive and IL-12 receptor-positive; in this way, the anti-PD1 portion of the fusion protein helps to focus IL12 activity on cells that are double positive for both PD1 and the IL-12 receptor.

The activity of these fusion proteins was assessed using human primary cells from healthy peripheral blood mononuclear cell (PBMC) donors. Purified CD4 T cells were activated in vitro and then stimulated with the different IL-12 H10 mutein/anti-PD1 fusion proteins to assess the levels of phosphorylation of STAT4, which is a readout for IL-12 activity. (Activated CD4 T cells have increased levels of IL-12 receptor and PD1.)

The half maximal effective concentration (EC₅₀) for the fusion proteins is show in Table 13. Each data column is for data from an individual PBMC donor.

TABLE 13

| | CD4 T cell | CD4 T cell |
|---|---|---|
| IL-12 H10 | activation assay | activation assay |
| muteln/ | PSTAT4 EC₅₀ | PSTAT4 EC₅₀ |
| anti-PD1 | (ug/ml); | (ug/ml); |
| fusion | Donor #1 | Donor #2 |
| H10/TPP-68807 | 0.1546 | |
| H10/TPP-76868 | 0.1388 | 0.9980 |
| H10/TPP-77658 | 0.2350 | 1.536 |
| H10/Isotype IgG | 19.17 | 95.6 |

As shown in Table 13 the EC₅₀ for the H10/anti-PD1 fusion molecules is lower than for an H10/Isotype IgG, indicating that the H10/anti-PD1 fusion molecules have targeted activity on PD1-positive cells.

The amino acid sequences for the polypeptides of the H10/TPP-76868 fusion protein are provided below in Table 14. This fusion is also referred to herein as the “H10868” fusion.

TABLE 14

| Description | Sequence |
|---|---|
| Heavy chain | EVQLVESGGGLVQPGGSLRLSCAASGFSFGDFDMRWFRQAPGKGLEWVGLIK |
| TPP-76868 | SRAYLEATEFAASVEGRFTISRDDAKNSAYLQMNSLRAEDTAVYYCTRDSYSS |
| | GLLDYWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPS |
| | NTKVDKKVEPKSCDKTHTCPPCPAPEAAGAPSVFLFPPKPKDTLMISRTPEVTC |
| | VVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPCREEMTKNQVSLSC |
| | AVKGFYPSDIAVEWESNGQPENNYKTTPPVLDSDGSFFLVSKLTVDKSRWQQ |
| | GNVFSCSVMHEALHNHYTQKSLSLSPGK (SEQ ID NO: 15) |
| Heavy | EVQLVESGGGLVQPGGSLRLSCAASGFSFGDFDMRWFRQAPGKGLEWVGLIK |
| Chain TPP- | SRAYLEATEFAASVEGRFTISRDDAKNSAYLQMNSLRAEDTAVYYCTRDSYSS |
| 76868 fused | GLLDYWGQGTLVTVSSASTKGPSVFPLAPSSKSTSGGTAALGCLVKDYFPEPV |
| to p35 of | TVSWNSGALTSGVHTFPAVLQSSGLYSLSSVVTVPSSSLGTQTYICNVNHKPS |
| H10 | NTKVDKKVEPKSCDKTHTCPPCPAPEAAGAPSVFLFPPKPKDTLMISRTPEVTC |
| | VVVDVSHEDPEVKFNWYVDGVEVHNAKTKPREEQYNSTYRVVSVLTVLHQDW |
| | LNGKEYKCKVSNKALPAPIEKTISKAKGQPREPQVCTLPPSREEMTKNQVSLW |
| | CLVKGFYPSDIAVEWESNGQPENNYKTTPPVLDSDGSFFLYSKLTVDKSRWQQ |
| | GNVFSCSVMHEALHNHYTQKSLSLSPGSGGGGSGGGGSGGGGRNLPVATPD |
| | PGMFPCLHHSQNLLRAVSNMLQKARQTLEFYPCTSEEIDHEDITKDKTSTVEAC |
| | LPLELTKNESCLNSRETSFITNGSCLASRKTSFMMALCLSSIYEDLKMYQVEFKT |
| | MNAKLLMDPKRQIFLDQNMLAVIDELMQALNFNSETVPQKSSLEEPDFAKTKIK |
| | LCILLHAFRIRAVTIDRVMSYLNAS (SEQ ID NO: 26) |
| Light chain | DIQMTQSPSSLSASVGDRVTITCRASQGISNYLAWFQQKPGKAPKRLIYAAQIP |
| TPP-76868 | GSGVPSRFSGSGSGTDFTLTISSLQPEDFATYYCLQHYSYPLTFGGGTKVEIKR |
| | TVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSFNRGEC |
| | (SEQ ID NO: 16) |
| P40 of H10 | IWELKKDVYVVELDWYPDAPGEMVVLTCDTPEEDGITWTLDQSSEVLGSGKTL |
| | TIQVKEFGDAGQYTCHKGGEVLSHSLLLLHKKEDGIWSTLILKDQKEPKNKTFL |
| | RCEAKNYSGRFTCWWLTTISTDLTFSVKSSRGSSDPQGVTCGAATLSAERVR |
| | GDNKEYEYSVECQEDSACPAAEESLPIEVMVDAVHKLKYENYTSSFFIRDIIKPD |
| | PPKNLQLKPLKNSRQVEVSWEYPDTWSTPHSYFSLTFCVQVQGGGGDRVFTD |
| | KTSATVICRKNASISVRAQDRYYSSSWSEWASVPCS (SEQ ID NO: 4) |

The amino acid sequences of the H10868 fusion shown in Table 14 include the following annotated features. In the Heavy Chain TPP-76868 sequence (SEQ ID NO: 15), there are effector null mutations in the Fc: L234A, L235A, and G237A (EU numbering; underlined), and mutations to form a ‘hole’ for a Knob-in-Hole structure: S354C, T366S, L368A, and Y407V (EU numbering; underlined). In the Heavy Chain TPP-76868 fused to p35 of H10 sequence (SEQ ID NO: 26), there are effector null mutations in the Fc: L234A, L235A, and G237A (EU numbering; underlined), mutations to form a “knob” for a Knob-in-Hole structure: Y349C and T366W (EU numbering; underlined), and a linker sequence [SGGGGSGGGGSGGGG (SEQ ID NO: 27)] connecting the heavy chain and p35 of H10.

### IL-12 H10 Mutein/Anti-PD1 TPP-77658 Fusion Protein

The amino acid sequences for the polypeptides of the H10/TPP-77658 fusion protein are provided below in Table 15. This fusion is also referred to herein as the “H10658” fusion.

TABLE 15

| Description | Sequence |
|---|---|
| Heavy chain | EVQLVESGGGLVQPGGSLRLSCAASGFSFGDFDMRWFRQAPGKGLEWVGTI |
| TPP-77658 | KSRAYLEATEFAASVEGRFTISRDDAKNSAYLQMNSLRAEDTAVYYCTRDAYS |
| | SGLLDYWGQGTLVTVSSASTKGPSVFPLAPSSKSTSGGTAALGQLVKDYFPE |
| | PVTVSWNSGALTSGVHTFPAVLQSSGLYSLSSVVTVPSSSLGTQTYICNVNHK |
| | PSNTKVDKKVEPKSCDKTHTCPPCPAPEAAGAPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPCREEMTKNQ |
| | VSLSCAVKGFYPSDIAVEWESNGQPENNYKTTPPVLDSDGSFFLVSKLTVDKS |
| | RWQQGNVFSCSVMHEALHNHYTQKSLSLSPGK (SEQ ID NO: 5) |
| Heavy chain | EVQLVESGGGLVQPGGSLRLSCAASGFSFGDFDMRWFRQAPGKGLEWVGTI |
| TPP-77658 | KSRAYLEATEFAASVEGRFTISRDDAKNSAYLQMNSLRAEDTAVYYCTRDAYS |
| fused to p35 | SGLLDYWGQGTLVTVSSASTKGPSVFPLAPSSKSTSGGTAALGCLVKDYFPE |
| of H10 | PVTVSWNSGALTSGVHTFPAVLQSSGLYSLSSVVTVPSSSLGTQTYICNVNHK |
| | PSNTKVDKKVEPKSCDKTHTCPPCPAPEAAGAPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVCTLPPSREEMTKNQ |
| | VSLWCLVKGFYPSDIAVEWESNGQPENNYKTTPPVLDSDGSFFLYSKLTVDKS |
| | RWQQGNVFSCSVMHEALHNHYTQKSLSLSPGSGGGGSGGGGSGGGGRNL |
| | PVATPDPGMFPCLHHSQNLLRAVSNMLQKARQTLEFYPCTSEEIDHEDITKDK |
| | TSTVEACLPLELTKNESCLNSRETSFITNGSCLASRKTSFMMALCLSSIYEDLK |
| | MYQVEFKTMNAKLLMDPKRQIFLDQNMLAVIDELMQALNFNSETVPQKSSLEE |
| | PDFAKTKIKLCILLHAFRIRAVTIDRVMSYLNAS (SEQ ID NO: 25) |
| Light chain | DIQMTQSPSSLSASVGDRVTITCRASQGISNYLAWFQQKPGKAPKRLIYAAQIP |
| TPP-77658 | GSGVPSRFSGSGSGTDFTLTISSLQPEDFATYYCLQHYSYPLTFGGGTKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNR |
| | GEC (SEQ ID NO: 6) |
| p40 of H10 | IWELKKDVYVVELDWYPDAPGEMVVLTCDTPEEDGITWTLDQSSEVLGSGKT |
| | LTIQVKEFGDAGQYTCHKGGEVLSHSLLLLHKKEDGIWSTLILKDQKEPKNKTF |
| | LRCEAKNYSGRFTCWWLTTISTDLTFSVKSSRGSSDPQGVTCGAATLSAERV |
| | RGDNKEYEYSVECQEDSACPAAEESLPIEVMVDAVHKLKYENYTSSFFIRDIIK |
| | PDPPKNLQLKPLKNSRQVEVSWEYPDTWSTPHSYFSLTFCVQVQGGGGDRV |
| | FTDKTSATVICRKNASISVRAQDRYYSSSWSEWASVPCS (SEQ ID NO: 4) |

The amino acid sequences of the H10658 fusion shown in Table 15 include the following annotated features. In the Heavy Chain TPP-77658 sequence (SEQ ID NO: 5), there are effector null mutations in the Fc: L234A, L235A, and G237A (EU numbering; underlined), and mutations to form a “hole” for a Knob-in-Hole structure. S354C, T366S, L368A, and Y407V (EU numbering; underlined). In the Heavy Chain TPP-77658 fused to p35 of H10 sequence (SEQ ID NO: 25), there are effector null mutations in the Fc: L234A, L235A, and G237A (EU numbering: underlined), mutations to form a “knob” for a Knob-in-Hole structure: Y349C and T366W (EU numbering; underlined), and a linker sequence [SGGGGSGGGGSGGGG (SEQ ID NO. 27)] connecting the heavy chain and p35 of H10.

### Example 4: Mouse Surrogate IL-12 Variant/Anti-PD1 Fusion Molecules

This example describes the generation of mouse surrogate IL-12 variant/anti-PD1 molecules, and related experiments. IL-12 biology is largely conserved in humans and mice. However, human IL-12 does not cross-react with the mouse IL-12 receptor, and cannot be used in preclinical mouse models. Therefore mouse surrogate IL-12 variants were generated in order to further assess the activity of IL-12 variants.

Two different mouse surrogate IL-12 variant/anti-PD1 molecules were developed. Both surrogate IL-12 variant/anti-PD1 molecules contain the same mouse IL-12 mutein, which was selected as having a similar level of attenuation as the human H10 IL12 mutein (described in Example 1). To facilitate production, the mouse IL-12 mutein was prepared as a single polypeptide, in which the IL-12 p40 and p35 subunits are covalently linked via a peptide linker (rather than expressing p40 and p35 as separate polypeptide chains). The sequence of the mouse IL12 mutein (containing the linked p40 and p35 subunits) is: MWELEKDVYWEVDWTPDAPGETVNLTCDTPEEDDITWTSDQRHGVIGSGKTLTITVKEFLDA GQYTCHKGGETLSHSHLLLHKKENGIWSTEILKNFKNKTFLKCEAPNYSGRFTCSWLVQRNMD LKFNIKSSSSSPDSRAVTCGMASLSAEKVTLDQRDYEKYSVSCQEDVTCPTAEETLPIELALEA RQQNKYENYSTSFFIRDIIKPDPPKNLQMKPLKNSQVEVSWEYPDSWSTPHSYFSLKFFVGGG EKMKETEEGCNQKGAFLVEKTSTEVQCKGGNVCVQAQDRYYNSSCSKWACVPCRVRSGGG GGGSRVIPVSGPARCLSQSRNLLKTTDDMVKTAREKLEFYPCTAEDIDHEDITRDQTSTLKTCL PLELHKNESCLATRETSSTTRGSCLPPQKTSLMMTLCLGSIYEDLKMYQTEFQAINAALQNHNH QQIILDKGMLVAIDELMQSLNHNGETLRQKPPVGEADPYRVKMKLCILLHAFSTRVVTINRVMG YLSSA (SEQ ID NO: 31). In this sequence, the p40 subunit is the N-terminal portion (i.e. first in the sequence), and the p35 subunit is the C-terminal portion (i.e. last in the sequence), and the p40 and p35 subunits are separated by a glycine-serine linker sequence (underlined) (GGGGGGS; SEQ ID NO: 32). The p40 and p35 portions of the polypeptide only contain the mature portions of the respective polypeptides (i.e. the leader sequences are not included). The mutein has the following mutations as compared to wild-type mouse IL-12 (each mutation is underlined in the above sequence; amino acid numbers are based on the mature polypeptide sequences): p40: GGG (to reduce heparin binding)[mouse p40 residues RIQRKK (amino acid numbers 251-256) were replaced with the sequence GGG]; p35: K34E, H35F, and S37P (each mutation for attenuation of IL-12 activity).

The two different mouse surrogate IL-12 variant/anti-PD1 fusion proteins contained the mouse IL-12 mutein described above, and either a PD1-blocking anti-mouse PD1 antibody, or a PD1-non-blocking anti-mouse PD1 antibody. Thus, the two mouse surrogate IL-12 variant/anti-PD1 fusions are: 1) mouse IL-12 mutein linked to anti-mouse PD1 blocking antibody [the fusion is also referred to as “mIL12 mutein-PD1(B)” or “PD1(B)-mIL12” ] and 2) mouse IL-12 mutein linked to anti-mouse PD1 non-blocking antibody [the fusion is also referred to as “mIL12 mutein-PD1(NB)” or “PD1(NB)-mIL12” ].

The activities of both mouse surrogate IL-12 mutein/anti-PD1 fusion proteins were compared in an ex-vivo CT26 tumor stimulation to assess whether there were differences in activity of the molecules arising out of the different anti-PD1 specificities (blocking vs non-blocking). CT26 tumors (murine colon carcinoma) were harvested from mice which had been implanted with CT26 cells. The tumors were processed to achieve single cell suspensions of tumor cells and other cells in the tumor microenvironment, which were then stimulated with one of four different fusion proteins: 1) mouse IL-12 wild-type linked to mouse Fc domain (“mIL12 wt-Fc”); 2) mouse IL-12 mutein linked to mouse Fc domain (“mIL12 mutein-Fc”); 3) mIL12 mutein-PD1(B); or 4) mIL12 mutein-PD1(NB). After 24 hours, activity of the different fusion proteins was assessed, as measured by the amount of interferon gamma (IFNg) present in the supernatant after 24 hours.

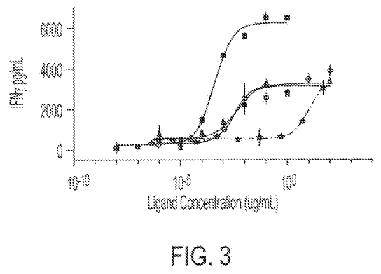

The results are shown in FIG. 3. In FIG. 3, the data points are depicted as follows: 1) mIL12 wt-Fc: solid line, solid squares; 2) mIL12 mutein-Fc: dashed line, stars; 3) mIL12 mutein-PD1(B): solid line, triangles; or 4) mIL12 mutein-PD1(NB): solid line, empty circles. As shown in FIG. 3, the activity of mIL12 mutein-PD1(B) and mIL12 mutein-PD1(NB) was similar, suggesting that PD1 blockade is not required for the PD1 targeted IL12 mutein activity. FIG. 3 also shows that mIL12 wt-Fc has greater activity than mIL12 mutein-PD1(B) and mIL12 mutein-PD1(NB), which is consistent with the wild-type IL-12 activity of mIL12 wt-Fc. FIG. 3 also shows that mIL12 mutein-Fc has less activity than mIL12 mutein-PD1(B) and mIL12 mutein-PD1(NB), which is consistent with the lack of targeting of mIL12 mutein-Fc.

The activity of a human IL-12 variant/anti-PD1 fusion protein containing the H10 IL-12 mutein was compared to that of the mouse surrogate IL-12 mutein/anti-PD1 fusion protein mIL12 mutein-PD1(B) in a matched assay set up using CD4 T cells, which were either acquired from healthy human donors or isolated from naïve mice splenocytes. The human IL-12 variant/anti-PD1 fusion protein used for this experiment contained the H10 IL-12 mutein (described in Example 1) linked to non-blocking anti-hPD1 antibody TPP-68807 (described in Example 2; it is a derivative of GBT-PD1-0013 and is closely related to TPP-77658). This fusion protein is referred to as “hIL12 mutein-hPD1(NB)” in this experiment.

The CD4 T cells were activated in vitro, and then stimulated with different fusion proteins as follows. Human CD4 T cells were stimulated with: 1) human IL-12 wildtype linked to human isotype (IgG1) control antibody (i.e. without specificity for PD1) (“hIL12 wt-isotype”); 2) human H10 IL-12 mutein (described in Example 1) fused to a human isotype (IgG1) control antibody (“hIL12 mutein-isotype”); and 3) hIL12 mutein-hPD1(NB). Mouse CD4 T cells were stimulated with: 1) mouse IL-12 wildtype fused to a human isotype (IgG1) control antibody (i.e. without specificity for PD1) (“mIL12 wt-isotype”); 2) mouse IL-12 mutein corresponding to human H10 fused to a human isotype (IgG1) control antibody (“mIL12 mutein-isotype”); and 3) mIL12 mutein-PD1(B) (described above). For each assay, pSTAT4 was measured by flow cytometry 60 minutes after stimulation of the T cells. The assay incorporated analysis of estimated PD1 receptor numbers per cell, and the pSTAT4 readouts were limited to cells having similar numbers of PD1 receptor per cell to permit for a more accurate comparison. The results are summarized below in Table 16.

TABLE 16

| | Human Fusion | Mouse Fusion |
|---|---|---|
| Activities Compared | Proteins | Proteins |
| Reduction in pSTAT4 production of 2) | ~23075-fold | ~23150- |
| mutein IL-12 fused to isotype mAb as | decrease | fold decrease |
| compared to 1) wild-type IL12 fused to | | |
| isotype mAb | | |
| Increase in pSTAT4 production of 3) | ~90-fold | ~100-fold |
| mutein IL-12 fused to anti-PD1 mAb | increase | increase |
| as compared to 2) mutein IL-12 fused | | |
| to isotype mAb | | |

As shown in Table 16, both the human and mouse IL12 muteins have similar levels of attenuation in activity, relative to the respective human or mouse wild-type IL12 (˜23,000-fold reduction). In addition, PD1-guided rescue of activity of the IL12 mutein (which is measured as a gain in activity of the IL12 mutein/anti-PD1 antibody fusion relative to the IL12 mutein/non-specific isotype antibody fusion), is also similar for both the human and mouse fusions (˜100-fold increase). Overall, this experiment indicates that the human hIL12 mutein-hPD1(NB) fusion protein and mouse surrogate mIL12 mutein-PD1(B) fusion protein are closely matched for both IL12 attenuation and activity rescued by the addition of the anti-PD1 antibody. Furthermore, in view of the experiment above showing comparable activity of the mIL12 mutein-PD1(B) and mIL12 mutein-PD1(NB) fusion proteins, it can be concluded that the hIL12 mutein-hPD1(NB) fusion protein has similar activity to both the mIL12 mutein-PD1(B) fusion protein and mIL12 mutein-PD1(NB) fusion protein in the respective experimental systems (human vs mouse).

### Example 5: In Vivo Efficacy Studies

Efficacy studies were performed in two syngeneic murine tumor models (MC38R and B16F10). MC38R is a murine colon adenocarcinoma cell line. B16F10 is a murine melanoma cell line. The molecules listed in Table 17 were used in these studies.

TABLE 17

| Molecule Name | Description |
|---|---|
| mIL12 mutein- | Mouse IL-12 mutein linked to anti-mouse PD1 |
| PD1(B) | blocking antibody |
| mIL12 mutein- | Mouse IL-12 mutein linked to anti-mouse PD1 |
| PD1(NB) | non-blocking antibody |
| mIL12 mutein- | Mouse IL-12 mutein linked to human IgG1 |
| isotype | isotype (control) antibody |
| mIL12 wt-isotype | Mouse IL-12 wildtype linked to human IgG1 |
| | isotype (control) antibody |
| mIL12 wt-PD1(B) | Mouse IL-12 wildtype linked to anti-mouse |
| | PD1 blocking antibody |
| Isotype | Human isotype IgG1 (control) antibody |
| mPD1(B) | Anti-mouse PD1 blocking antibody |
| mPD1 F2 | Anti-mouse PD1 antibody, clone F2 |
| mPD1 RMP1-14 | Anti-mouse PD1 antibody, clone RMP1-14 |

### MC38R Experiment 1

Method: 500,000 MC38R tumor cells were implanted subcutaneously into female C57BL/6 mice. After 10 days, mice were randomized into treatment groups with average tumor volumes of 44-92 mm3. Mice were treated with a single subcutaneous dose of 1) mIL12 mutein-PD1(NB); 2) mIL12 mutein-isotype; 3) mouse isotype; or 4) mPD1(B). The mIL12 mutein-PD1(NB) and mIL12 mutein-isotype were administered at 0.05 mg/kg, 0.17 mg/kg, or 0.5 mg/kg, and the mouse isotype and mPD1(B) were administered at 0.5 mg/kg on the day after randomization. Measurements were taken twice weekly for tumor volume and body weight until the end of the study when the first mouse reached a tumor volume of 2000 m3. There were 10 mice/treatment group.

Results: In this experiment, a single dose of mIL12 mutein-PD1(B) at 0.05 mg/kg, 0.17 mg/kg, or 0.5 mg/kg administered subcutaneously elicited strong, dose-dependent tumor growth inhibition (TGI) (61%, 76%, and 92%, respectively), whereas administration of the mIL12 mutein-isotype at the same dose levels did not induce significant TGI (−25%, −13%, and 45%, respectively). Moreover, administration of the plain mPD1(B) antibody (i.e. not fused to an IL12 mutein) also did not induce significant TGI (12%). The lack of TGI of the mPD1(B) antibody treatment group was expected, since it was dosed at a 20-fold lower amount and less frequently (a single dose instead of every 3 days, 3 times or once a week, 2 times) than therapeutic PD1 antagonist antibodies are normally dosed. No significant body weight loss (BWL) was observed at any dose level tested for any of these molecules.

### MC38R Experiment 2

Method: 500,000 MC38R tumor cells were implanted subcutaneously into female C57BL/6 mice. After 10 days, mice were randomized into treatment groups with average tumor volumes of 31-131 mm3. Mice were treated with a single subcutaneous dose of 1) mIL12 wt-isotype; 2) mIL12 wt-PD1(B); 3) mIL12 mutein-isotype; 4) mIL12 mutein-PD1(B); 5) mouse isotype or 6) mPD1(B) at 0.5 mg/kg on the day after randomization. Measurements were taken twice weekly for tumor volume and body weight until the end of the study when the first mouse reached a tumor volume of 2000 m3. There were 3-10 mice/treatment group.

Results: Each of mIL12 mutein-PD1(B), mIL12 wt-isotype, and mIL12 wt-PD1(B) elicited strong, significant TGI when administered as a single dose of 0.5 mg/kg subcutaneously relative to isotype-treated animals: 87% TGI for mIL12 mutein-PD1(B), 96% TGI for mIL12 wt-isotype, and 70% TGI for mIL12 wt-PD1(B). Treatment with mIL12 mutein-isotype did not elicit significant TGI (14%). Mice treated with mIL12 mutein-PD1(B) exhibited no BWL, but mice treated with either mIL12 wt-isotype or mIL12 wt-PD1(B) showed significant BWL on day 6 post-dose and averaging BWL of 21% and 19%, respectively.

These results show that mIL12 mutein-PD1(B) can elicit strong TGI without BWL; this is in contrast to mIL12 wt-isotype and mIL12 wt-PD1(B) which both elicit strong TGI but which induce significant BWL.

### MC38R Experiment 3

This experiment used MC38R B2M KO cells. In these tumor cells, the B2M gene is deleted, so the tumor cells are unable to load antigen onto major histocompatibility (MHC) I complexes. This renders the tumors MHC I low or negative and resistant to direct CD8 T cell killing. Accordingly, these tumor cells are also resistant to PD(L)1 therapy.

Method: 500,000 MC38R B2M KO tumor cells were implanted subcutaneously into female C57BL/6 mice. After 7 days, mice were randomized into treatment groups with average tumor volumes of 52-91 mm3. Starting on the day of randomization (d0), mice were treated with 1) mIL12 mutein-PD1(NB), 2) mPD1 F2, 3) mPD1 RMP1-14, or 4) mouse isotype. mIL12 mutein-PD1(NB) was administered once subcutaneously at 0.5 mg/kg, mPD1 F2 was administered intraperitoneally every 3 days, 3 times (Q3D×3) at 10 mg/kg, mPD1 RMP1-14 was administered once a week, two times (QW×2) at 10 mg/kg, and mouse isotype was administered QW×2 at 10 mg/kg. Measurements were taken twice weekly for tumor volume and body weight until the end of the study when the first mouse reached a tumor volume of 2000 m3. There were 10 mice/treatment group.

Results: The single dose of mIL12 mutein-PD1(NB) elicited strong, significant TGI (75%) relative to the mouse isotype. In contrast, mPD1 F2 and mPD1 RMP1-14 failed to elicit significant TGI against these tumor cells (−23% and 5% relative to the mouse isotype, respectively). No BWL was observed in any group.

These results show that mIL12 mutein-PD1(NB) is effective at inducing TGI in a PD(L)1-resistant tumor model.

### B16F10 Experiment 1

Method: 500,000 B16F10 tumor cells were implanted subcutaneously into female C57BL/6 mice. After 10 days, mice were randomized into treatment groups with average tumor volumes of 52-111 mm3. On the day of randomization (d0), mice were treated with a single subcutaneous dose of 1) mIL12 mutein-PD1(B), 2) mIL12 mutein-PD1(NB), or 3) mouse isotype. mIL12 mutein-PD1(B) and mIL12 mutein-PD1(NB) were each administered once subcutaneously at 0.5 mg/kg and 1.5 mg/kg (different treatment groups), and mouse isotype was administered at 1.5 mg/kg.

Results: A single dose of mIL12 mutein-PD1(B) elicited strong, dose dependent TGI (0.5 mg/kg: 67% TGI; 1.5 mg/kg: 87% TGI). In addition, a single dose of mIL12 mutein-PD1(NB) also elicited strong, dose dependent TGI (0.5 mg/kg; 76% TGI; 1.5 mg/kg: 78% TGI). No BWL was observed in any group.

These results show the effectiveness of the mIL12 mutein-PD1(B) and mIL12 mutein-PD1(NB) molecules at inducing TGI in the B16F10 tumor model. In addition, these results show that the effectiveness of the mIL12 mutein-PD1(B) and mIL12 mutein-PD1(NB) molecules is not dependent on antagonizing the interaction between PD1 and PDL1.

1. An isolated human interleukin 12 (IL-12) variant comprising an amino acid substitution at position Y167 of SEQ ID NO: 1 (IL-12 p35 subunit) and position D93 of SEQ ID NO: 2 (IL-12 p40 subunit).

2. The IL-12 variant of claim 1, wherein the Y167 substitution is Y167A

3. The IL-12 variant of claim 1, wherein the D93 substitution is D93L

4. The IL-12 variant of claim 1, wherein the Y167 substitution is Y167A and the D93 substitution is D93L.

5. The IL-12 variant of claim 1, wherein the p40 subunit further comprises one or more mutations to reduce the binding of IL-12 to heparin.

6. The IL-12 variant of claim 5, wherein the mutations to reduce the binding of IL-12 to heparin comprise the substitutions K258G, S259G, and K260G, and deletions of R261, E262, K263, and K264 of SEQ ID NO: 2.

7. The IL-12 variant of claim 1, comprising one or both of i) a polypeptide comprising the amino acid sequence of SEQ ID NO: 3 (variant IL-12 p35 subunit) and ii) a polypeptide comprising the amino acid sequence of SEQ ID NO: 4 (variant IL-12 p40 subunit).

8. An isolated human interleukin 12 (IL-12) variant comprising an amino acid substitution at one or more of positions: F39 of SEQ ID NO: 1 (IL-12 p35 subunit), 152 of SEQ ID NO: 1, Y167 of SEQ ID NO: 1, K85 of SEQ ID NO: 2 (IL-12 p40 subunit) and D93 of SEQ ID NO: 2.

9. The IL-12 variant of claim 8, wherein the F39 substitution is F39R or F39A, the I52 substitution is I52E, I52R, or I52H, the Y167 substitution is Y167A, the K85 substitution is K85E, and the D93 substitution is D93L.

10. The IL-12 variant of claim 8, wherein the p40 subunit further comprises one or more mutations to reduce the binding of IL-12 to heparin.

11. The IL-12 variant of claim 1, wherein the IL-12 variant has reduced activity as compared to wild-type human IL-12.

12. An isolated antibody that binds to PD1 and comprises:
i) a heavy chain variable region (VH) and a light chain variable region (VL), wherein the VH CDR1 comprises the amino acid sequence of SEQ ID NOs: 9, 35, or 36, the VH CDR2 comprises the amino acid sequence of SEQ ID NO: 10 or 37, the VH CDR3 comprises the amino acid sequence of SEQ ID NO: 11, the VL CDR1 comprises the amino acid sequence of SEQ ID NO: 12, the VL CDR2 comprises the amino acid sequence of SEQ ID NO: 13, and the VL CDR3 comprises the amino acid sequence of SEQ ID NO: 14;
ii) a heavy chain variable region (VH) and a light chain variable region (VL), wherein the VH CDR1 comprises the amino acid sequence of SEQ ID NO: 19, 35, or 36, the VH CDR2 comprises the amino acid sequence of SEQ ID NO: 20 or 37, the VH CDR3 comprises the amino acid sequence of SEQ ID NO: 21, the VL CDR1 comprises the amino acid sequence of SEQ ID NO: 22, the VL CDR2 comprises the amino acid sequence of SEQ ID NO: 23, and the VL CDR3 comprises the amino acid sequence of SEQ ID NO: 24: or
iii) a heavy chain variable region (VH) and a light chain variable region (VL), wherein the VH CDR1 comprises the amino acid sequence of SEQ ID NO: 9, 35, or 36, the VH CDR2 comprises the amino acid sequence of SEQ ID NO: 38 or 39, the VH CDR3 comprises the amino acid sequence of SEQ ID NO: 21, the VL CDR1 comprises the amino acid sequence of SEQ ID NO: 12, the VL CDR2 comprises the amino acid sequence of SEQ ID NO: 40, and the VL CDR3 comprises the amino acid sequence of SEQ ID NO: 41.

13. The antibody of claim 12, wherein the VH comprises the amino acid sequence of SEQ ID NO: 7 and the VL comprises the amino acid sequence of SEQ ID NO: 8.

14. The antibody of claim 13, wherein the antibody comprises a heavy chain comprising the amino acid sequence of SEQ ID NO: 5, 51 or 52 and a light chain comprising the amino acid sequence of SEQ ID NO: 6, wherein the C-terminal lysine of SEQ ID NO: 5, 51, or 52 is optional.

15-16. (canceled)

17. The antibody of claim 12, wherein the antibody does not block the binding of PDL1 to PD1.

18. An isolated fusion protein comprising a human interleukin 12 (IL-12) variant of claim 1 linked to an anti-PD1 antibody.

19. The fusion protein of claim 18, wherein the anti-PD1 antibody comprises:
i) a heavy chain variable region (VH) and a light chain variable region (VL), wherein the VH CDR1 comprises the amino acid sequence of SEQ ID NOs: 9, 35, or 36, the VH CDR2 comprises the amino acid sequence of SEQ ID NO: 10 or 37, the VH CDR3 comprises the amino acid sequence of SEQ ID NO: 11, the VL CDR1 comprises the amino acid sequence of SEQ ID NO: 12, the VL CDR2 comprises the amino acid sequence of SEQ ID NO: 13, and the VL CDR3 comprises the amino acid sequence of SEQ ID NO: 14;
ii) a heavy chain variable region (VH) and a light chain variable region (VL), wherein the VH CDR1 comprises the amino acid sequence of SEQ ID NO: 19, 35, or 36, the VH CDR2 comprises the amino acid sequence of SEQ ID NO: 20 or 37, the VH CDR3 comprises the amino acid sequence of SEQ ID NO: 21, the VL CDR1 comprises the amino acid sequence of SEQ ID NO: 22, the VL CDR2 comprises the amino acid sequence of SEQ ID NO: 23, and the VL CDR3 comprises the amino acid sequence of SEQ ID NO: 24: or
iii) a heavy chain variable region (VH) and a light chain variable region (VL), wherein the VH CDR1 comprises the amino acid sequence of SEQ ID NO: 9, 35, or 36, the VH CDR2 comprises the amino acid sequence of SEQ ID NO: 38 or 39, the VH CDR3 comprises the amino acid sequence of SEQ ID NO: 21, the VL CDR1 comprises the amino acid sequence of SEQ ID NO: 12, the VL CDR2 comprises the amino acid sequence of SEQ ID NO: 40, and the VL CDR3 comprises the amino acid sequence of SEQ ID NO: 41.

20-21. (canceled)

22. An isolated fusion protein comprising a human interleukin 12 (IL-12) variant and an anti-PD1 antibody, wherein the fusion protein comprises the polypeptides of 1 SEQ ID NOs: 5, 25, 6, and 4 or the polypeptides of ii) SEQ ID NOs: 15, 26, 16, and 4.

23. (canceled)

24. An isolated polynucleotide or polynucleotides comprising one or more nucleotide sequences encoding the IL-12 variants of claim 1.

25. The isolated polynucleotide or polynucleotides of claim 24, wherein the one or more nucleotide sequences comprise a nucleotide sequence of SEQ ID NO: 44 and a nucleotide sequence of SEQ ID NO: 45.

26. An isolated polynucleotide or polynucleotides comprising one or more nucleotide sequences encoding the VH, VL, or both of an antibody that binds PD1, wherein the polynucleotide(s) comprise the VH nucleic acid sequence of SEQ ID NO: 46, the VL nucleic acid sequence of SEQ ID NO: 47, or both the VH nucleic acid sequence of SEQ ID NO: 46 and the VL nucleic acid sequence of SEQ ID NO: 47.

27. An isolated polynucleotide or polynucleotides comprising one or more nucleotide sequences encoding any one or more of the heavy chain, light chain, IL-12 p40 subunit, or heavy chain-IL12 p35 fusion polypeptide of a fusion protein comprising a human IL-12 variant and an anti-PD1 antibody, wherein the polynucleotide(s) comprise the heavy chain nucleic acid sequence of SEQ ID NO: 48, the light chain nucleic acid sequence of SEQ ID NO: 50, the IL-12 p40 subunit nucleic acid sequence of SEQ ID NO: 45, the heavy chain-IL12 p35 fusion polypeptide nucleic acid sequence of SEQ ID NO: 49 or each of the heavy chain nucleic acid sequence of SEQ ID NO: 48, the light chain nucleic acid sequence of SEQ ID NO: 50, the IL-12 p40 subunit nucleic acid sequence of SEQ ID NO: 45, and the heavy chain-IL12 p35 fusion polypeptide nucleic acid sequence of SEQ ID NO: 49.

28. An isolated polynucleotide or polynucleotides comprising one or more nucleotide sequences encoding any one or more of the heavy chain, light chain, IL-12 p40 subunit, or heavy chain-IL12 p35 fusion polypeptide of a fusion protein comprising a human IL-12 variant and an anti-PD1 antibody, wherein the polynucleotide(s) comprise the heavy chain encoding nucleic acid sequence of the insert of the plasmid deposited with the ATCC and having ATCC Accession Number PTA-127517, the light chain encoding nucleic acid sequence of the insert of the plasmid deposited with the ATCC and having ATCC Accession Number PTA-127519, the IL-12 p40 subunit encoding nucleic acid sequence of the insert of the plasmid deposited with the ATCC and having ATCC Accession Number PTA-127520, the heavy chain-IL12 p35 fusion polypeptide encoding nucleic acid sequence of the insert of the plasmid deposited with the ATCC and having ATCC Accession Number PTA-127518, or each of the heavy chain encoding nucleic acid sequence of the insert of the plasmid deposited with the ATCC and having ATCC Accession Number PTA-127517, the light chain encoding nucleic acid sequence of the insert of the plasmid deposited with the ATCC and having ATCC Accession Number PTA-127519, the IL-12 p40 subunit encoding nucleic acid sequence of the insert of the plasmid deposited with the ATCC and having ATCC Accession Number PTA-127520, and the heavy chain-IL12 p35 fusion polypeptide encoding nucleic acid sequence of the insert of the plasmid deposited with the ATCC and having ATCC Accession Number PTA-127518.

29. A vector comprising the polynucleotide or polynucleotides of claim 24.

30. An isolated host cell comprising the polynucleotide or polynucleotides of claim 24.

31. A method of producing an IL-12 variant comprising culturing the host cell of claim 30 under conditions that result production of the IL-12 variant, and optionally further recovering the IL-12 variant.

32. A pharmaceutical composition comprising the IL-12 variant of claim 1 and a pharmaceutically acceptable carrier.

33. A method of treating cancer in a subject, the method comprising administering to the subject in need thereof a therapeutically effective amount of the pharmaceutical composition of claim 32.

34. (canceled)

35. The pharmaceutical composition of claim 33 wherein the cancer is bladder cancer, breast cancer, clear cell kidney cancer, head/neck squamous cell carcinoma [squamous cell carcinoma of the head and neck (SCCHN)], lung squamous cell carcinoma, lung adenocarcinoma, malignant melanoma, non-small-cell lung cancer (NSCLC), ovarian cancer, pancreatic cancer, prostate cancer, renal cell carcinoma (RCC), small-cell lung cancer (SCLC), triple negative breast cancer, urothelial cancer, acute lymphoblastic leukemia (ALL), acute myeloid leukemia (AML), chronic lymphocytic leukemia (CLL), chronic myeloid leukemia (CML), diffuse large B-cell lymphoma (DLBCL), follicular lymphoma, Hodgkin's lymphoma (HL), mantle cell lymphoma (MCL), multiple myeloma (MM), myeloid cell leukemia-1 protein (Mcl-1), myelodysplastic syndrome (MDS), non-Hodgkin's lymphoma (NHL), small lymphocytic lymphoma (SLL), endometrial cancer, B-cell acute lymphoblastic leukemia, colorectal cancer (CRC), glioblastoma, uterine cancer, cervical cancer, penile cancer, gastric cancer (GC) or non-melanoma skin cancer.

36. (canceled)


SEQUENCE LISTING (56 sequences)

SEQ ID NO: 1 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 197 aa, synthetic construct
  REGION 1..197: note = Synthetic
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 4 - protein, 302 aa, synthetic construct
  REGION 1..302: note = Synthetic
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTLILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGGGGDRVFTDKTSATVICRKNASISVRAQDRYYSSSWSEWASVPCS

SEQ ID NO: 5 - protein, 451 aa, synthetic construct
  REGION 1..451: note = Synthetic
EVQLVESGGGLVQPGGSLRLSCAASGFSFGDFDMRWFRQAPGKGLEWVGTIKSRAYLEATEFAASVEGRFTISRDDAKNSAYLQMNSLRAEDTAVYYCTRDAYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 6 - protein, 214 aa, synthetic construct
  REGION 1..214: note = Synthetic
DIQMTQSPSSLSASVGDRVTITCRASQGISNYLAWFQQKPGKAPKRLIYAAQIPGSGVPSRFSGSGSGTDFTLTISSLQPEDFATYYCLQHYSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 121 aa, synthetic construct
  REGION 1..121: note = Synthetic
EVQLVESGGGLVQPGGSLRLSCAASGFSFGDFDMRWFRQAPGKGLEWVGTIKSRAYLEATEFAASVEGRFTISRDDAKNSAYLQMNSLRAEDTAVYYCTRDAYSSGLLDYWGQGTLVTVSS

SEQ ID NO: 8 - protein, 107 aa, synthetic construct
  REGION 1..107: note = Synthetic
DIQMTQSPSSLSASVGDRVTITCRASQGISNYLAWFQQKPGKAPKRLIYAAQIPGSGVPSRFSGSGSGTDFTLTISSLQPEDFATYYCLQHYSYPLTFGGGTKVEIK

SEQ ID NO: 9 - protein, 10 aa, synthetic construct
  REGION 1..10: note = Synthetic
GFSFGDFDMR

SEQ ID NO: 10 - protein, 19 aa, synthetic construct
  REGION 1..19: note = Synthetic
TIKSRAYLEATEFAASVEG

SEQ ID NO: 11 - protein, 10 aa, synthetic construct
  REGION 1..10: note = Synthetic
DAYSSGLLDY

SEQ ID NO: 12 - protein, 11 aa, synthetic construct
  REGION 1..11: note = Synthetic
RASQGISNYLA

SEQ ID NO: 13 - protein, 7 aa, synthetic construct
  REGION 1..7: note = Synthetic
AAQIPGS

SEQ ID NO: 14 - protein, 9 aa, synthetic construct
  REGION 1..9: note = Synthetic
LQHYSYPLT

SEQ ID NO: 15 - protein, 451 aa, synthetic construct
  REGION 1..451: note = Synthetic
EVQLVESGGGLVQPGGSLRLSCAASGFSFGDFDMRWFRQAPGKGLEWVGLIKSRAYLEATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 16 - protein, 214 aa, synthetic construct; identical to SEQ ID NO: 6
  REGION 1..214: note = Synthetic

SEQ ID NO: 17 - protein, 121 aa, synthetic construct
  REGION 1..121: note = Synthetic
EVQLVESGGGLVQPGGSLRLSCAASGFSFGDFDMRWFRQAPGKGLEWVGLIKSRAYLEATEFAASVEGRFTISRDDAKNSAYLQMNSLRAEDTAVYYCTRDSYSSGLLDYWGQGTLVTVSS

SEQ ID NO: 18 - protein, 107 aa, synthetic construct; identical to SEQ ID NO: 8
  REGION 1..107: note = Synthetic

SEQ ID NO: 19 - protein, 10 aa, synthetic construct
  REGION 1..10: note = Synthetic
GFSFGDFDMR

SEQ ID NO: 20 - protein, 19 aa, synthetic construct
  REGION 1..19: note = Synthetic
LIKSRAYLEATEFAASVEG

SEQ ID NO: 21 - protein, 10 aa, synthetic construct
  REGION 1..10: note = Synthetic
DSYSSGLLDY

SEQ ID NO: 22 - protein, 11 aa, synthetic construct
  REGION 1..11: note = Synthetic
RASQGISNYLA

SEQ ID NO: 23 - protein, 7 aa, synthetic construct
  REGION 1..7: note = Synthetic
AAQIPGS

SEQ ID NO: 24 - protein, 9 aa, synthetic construct
  REGION 1..9: note = Synthetic
LQHYSYPLT

SEQ ID NO: 25 - protein, 662 aa, synthetic construct
  REGION 1..662: note = Synthetic
EVQLVESGGGLVQPGGSLRLSCAASGFSFGDFDMRWFRQAPGKGLEWVGTIKSRAYLEATEFAASVEGRFTISRDDAKNSAYLQMNSLRAEDTAVYYCTRDAYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 26 - protein, 662 aa, synthetic construct
  REGION 1..662: note = Synthetic
EVQLVESGGGLVQPGGSLRLSCAASGFSFGDFDMRWFRQAPGKGLEWVGLIKSRAYLEATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGSGGGGSGGGGSGGGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFAKTKIKLCILLHAFRIRAVTIDRVMSYLNAS

SEQ ID NO: 27 - protein, 15 aa, synthetic construct
  REGION 1..15: note = Synthetic
SGGGGSGGGGSGGGG

SEQ ID NO: 28 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 30 - protein, 7 aa, Homo sapiens
KSKREKK

SEQ ID NO: 31 - protein, 510 aa, synthetic construct
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVGGGEKMKETEEGCNQKGAFLVEKTSTEVQCKGGNVCVQAQDRYYNSSCSKWACVPCRVRSGGGGGGSRVIPVSGPARCLSQSRNLLKTTDDMVKTAREKLEFYPCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 32 - protein, 7 aa, synthetic construct
GGGGGGS

SEQ ID NO: 33 - protein, 121 aa, synthetic construct
EVQLVESGGGLVQPGGSLRLSCAASGFSFGDFDMRWFRQAPGKGLEWVGLIKSKAYRYATEFAASVEGRFTISRDDAKNSAYLQMNSLRAEDTAVYYCTRDSYSSGLLDYWGQGTLVTVSS

SEQ ID NO: 34 - protein, 107 aa, synthetic construct
DIQMTQSPSSLSASVGDRVTITCRASQGISNYLAWFQQKPGKAPKRLIYAASIPGSGVPSRFSGSGSGTDFTLTISSLQPEDFATYYCLQHKSYPLTFGGGTKVEIK

SEQ ID NO: 35 - protein, 7 aa, synthetic construct
GFSFGDF

SEQ ID NO: 36 - protein, 5 aa, synthetic construct
DFDMR

SEQ ID NO: 37 - protein, 8 aa, synthetic construct
KSRAYLEA

SEQ ID NO: 38 - protein, 8 aa, synthetic construct
KSKAYRYA

SEQ ID NO: 39 - protein, 19 aa, synthetic construct
LIKSKAYRYATEFAASVEG

SEQ ID NO: 40 - protein, 7 aa, synthetic construct
AASIPGS

SEQ ID NO: 41 - protein, 9 aa, synthetic construct
LQHKSYPLT

SEQ ID NO: 42 - protein, 451 aa, synthetic construct
EVQLVESGGGLVQPGGSLRLSCAASGFSFGDFDMRWFRQAPGKGLEWVGLIKSKAYRYATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 214 aa, synthetic construct
DIQMTQSPSSLSASVGDRVTITCRASQGISNYLAWFQQKPGKAPKRLIYAASIPGSGVPSRFSGSGSGTDFTLTISSLQPEDFATYYCLQHKSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 591 bp, synthetic construct
  source 1..591: mol_type = other DNA
cggaacctccccgtcgcaactcctgacccggggatgttcccttgtctgcaccatagccagaacctgttgagagccgtgtccaacatgctccagaaagcccggcagactcttgagttttacccatgcacctccgaagaaatcgaccatgaagatattaccaaagacaagacctccactgtggaagcgtgcttgccacttgagctgactaagaacgaaagctgcctgaactcccgggagacttctttcatcaccaatggttcctgccttgcgtcccgcaagacctcttttatgatggccctgtgcctgtcaagcatatacgaagatctgaagatgtaccaagtcgagttcaagaccatgaatgcgaagctccttatggaccctaagcggcagatcttcctggatcagaacatgctggctgtgatcgacgagctgatgcaggctctgaacttcaacagcgagacagtgccgcaaaagtccagcctggaagaacccgacttcgccaagaccaagatcaagctgtgcattctgctgcacgcattcaggatcagggcagtcactattgacagagtgatgtcctacctgaacgccagc

SEQ ID NO: 45 - DNA, 906 bp, synthetic construct
  source 1..906: mol_type = other DNA
atctgggaactgaagaaagatgtgtacgtggtcgaacttgactggtaccccgatgcgcctggagagatggtcgtgctgacttgcgatacgcctgaggaagatggaatcacttggactctcgaccagtcgtccgaagtcctcgggtcgggaaagaccctgacaatccaggtcaaggagttcggggacgccggacagtacacttgccacaagggcggagaagtgctgtcacactccctgctgctcctccacaagaaggaagatggcatctggtccactctgatcctcaaggaccagaaggagccgaagaacaagactttcctgcgctgcgaggccaagaactactccggacggttcacgtgttggtggctgaccaccattagcaccgacctgaccttctccgtgaagtccagccgggggagcagcgacccgcagggagtgacctgtggcgccgcgaccctctccgctgagcgcgtgcggggagacaacaaggaatatgagtacagcgtggagtgtcaggaagattccgcctgtcctgctgccgaagagtcgctgccaattgaagtgatggtcgatgccgtgcataagttgaaatacgagaactacacctcgtcgttcttcatccgggacatcattaagcccgacccgcccaagaacttgcagctgaagcccctgaagaactcgagacaggtcgaagtgtcctgggagtatcccgacacctggtccaccccccattcgtacttctcgctgactttctgtgtgcaagtgcagggtggcggcggggacagggtgttcaccgataagacctcagccactgtgatttgccgcaagaacgcgtcaatttcagtcagggcccaggatcggtattactcctcgtcatggtccgaatgggcctccgtgccctgctcg

SEQ ID NO: 46 - DNA, 363 bp, synthetic construct
  source 1..363: mol_type = other DNA
gaggtgcaactggtggaaagcggaggaggcctggtgcagcccggcggatctctgcggctgtcttgtgccgcttctggcttcagcttcggcgacttcgacatgcggtggtttagacaggcccctggcaagggcctcgagtgggtgggcaccatcaaaagcagagcttatctggaagccaccgagttcgccgccagcgtggaaggcagattcaccatcagccgggacgacgccaagaactccgcctacctgcagatgaacagcctgagagccgaggacaccgccgtgtactactgcaccagagatgcctacagcagcggcctgctggattactggggccagggcacactggtcacagtgtccagc

SEQ ID NO: 47 - DNA, 321 bp, synthetic construct
  source 1..321: mol_type = other DNA
gatatccagatgacccagagccctagctctctgagcgccagcgtgggcgacagagtgaccatcacctgtagagcctctcagggcatctccaactacctggcttggttccagcagaaacctggcaaggcccctaagcggctgatctacgccgctcagatcccaggcagcggcgtccccagcagattcagcggctccggctctggcaccgacttcaccctgaccattagcagcctgcaacctgaggactttgccacatattactgcctgcagcactacagctaccccctgacattcggcggaggaacaaaggtggaaatcaag

SEQ ID NO: 48 - DNA, 1353 bp, synthetic construct
  source 1..1353: mol_type = other DNA
gaggtgcaactggtggaaagcggaggaggcctggtgcagcccggcggatctctgcggctgtcttgtgccgcttctggcttcagcttcggcgacttcgacatgcggtggtttagacaggcccctggcaagggcctcgagtgggtgggcaccatcaaaagcagagcttatctggaagccaccgagttcgccgccagcgtggaaggcagattcaccatcagccgggacgacgccaagaactccgcctacctgcagatgaacagcctgagagccgaggacaccgccgtgtactactgcaccagagatgcctacagcagcggcctgctggattactggggccagggcacactggtcacagtgtccagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggaggagatgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgttagcaagctcaccgtggacaagagcaggtggcagcaggggaacgtcttctcatgctccgtgatgcatgaggctctgcacaaccactacacgcagaagagcctctccctgtcccccggaaaa

SEQ ID NO: 49 - DNA, 1986 bp, synthetic construct
  source 1..1986: mol_type = other DNA
gaggtgcaactggtggaaagcggaggaggcctggtgcagcccggcggatctctgcggctgtcttgtgccgcttctggcttcagcttcggcgacttcgacatgcggtggtttagacaggcccctggcaagggcctcgagtgggtgggcaccatcaaaagcagagcttatctggaagccaccgagttcgccgccagcgtggaaggcagattcaccatcagccgggacgacgccaagaactccgcctacctgcagatgaacagcctgagagccgaggacaccgccgtgtactactgcaccagagatgcctacagcagcggcctgctggattactggggccagggcacactggtcacagtgtccagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtcccccggaagcggtggaggaggttccggtggcggaggttcaggtggcggagggcggaacctccccgtcgcaactcctgacccggggatgttcccttgtctgcaccatagccagaacctgttgagagccgtgtccaacatgctccagaaagcccggcagactcttgagttttacccatgcacctccgaagaaatcgaccatgaagatattaccaaagacaagacctccactgtggaagcgtgcttgccacttgagctgactaagaacgaaagctgcctgaactcccgggagacttctttcatcaccaatggttcctgccttgcgtcccgcaagacctcttttatgatggccctgtgcctgtcaagcatatacgaagatctgaagatgtaccaagtcgagttcaagaccatgaatgcgaagctccttatggaccctaagcggcagatcttcctggatcagaacatgctggctgtgatcgacgagctgatgcaggctctgaacttcaacagcgagacagtgccgcaaaagtccagcctggaagaacccgacttcgccaagaccaagatcaagctgtgcattctgctgcacgcattcaggatcagggcagtcactattgacagagtgatgtcctacctgaacgccagc

SEQ ID NO: 50 - DNA, 642 bp, synthetic construct
  source 1..642: mol_type = other DNA
gatatccagatgacccagagccctagctctctgagcgccagcgtgggcgacagagtgaccatcacctgtagagcctctcagggcatctccaactacctggcttggttccagcagaaacctggcaaggcccctaagcggctgatctacgccgctcagatcccaggcagcggcgtccccagcagattcagcggctccggctctggcaccgacttcaccctgaccattagcagcctgcaacctgaggactttgccacatattactgcctgcagcactacagctaccccctgacattcggcggaggaacaaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 51 - protein, 451 aa, synthetic construct
EVQLVESGGGLVQPGGSLRLSCAASGFSFGDFDMRWFRQAPGKGLEWVGTIKSRAYLEATEFAASVEGRFTISRDDAKNSAYLQMNSLRAEDTAVYYCTRDAYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 451 aa, synthetic construct
EVQLVESGGGLVQPGGSLRLSCAASGFSFGDFDMRWFRQAPGKGLEWVGTIKSRAYLEATEFAASVEGRFTISRDDAKNSAYLQMNSLRAEDTAVYYCTRDAYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 451 aa, synthetic construct
EVQLVESGGGLVQPGGSLRLSCAASGFSFGDFDMRWFRQAPGKGLEWVGLIKSRAYLEATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 451 aa, synthetic construct
EVQLVESGGGLVQPGGSLRLSCAASGFSFGDFDMRWFRQAPGKGLEWVGLIKSRAYLEATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 451 aa, synthetic construct
EVQLVESGGGLVQPGGSLRLSCAASGFSFGDFDMRWFRQAPGKGLEWVGLIKSKAYRYATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 451 aa, synthetic construct
EVQLVESGGGLVQPGGSLRLSCAASGFSFGDFDMRWFRQAPGKGLEWVGLIKSKAYRYATEFAASVEGRFTISRDDAKNSAYLQMNSLRAEDTAVYYCTRDSYSSGLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK